SEQUENCE LISTING (1 sequence)

SEQ ID NO: 1 - protein, 204 aa, synthetic construct
MHHHHHHHHENLYFQSGVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEV

(19) United States
(12) Patent Application Publication
(10) Pub. No.: US 2025/0333398 A1
(43) Pub. Date: Oct. 30, 2025

(54) MONOARYL AND HETARYL SUBSTITUTED INDAZOLES AND BENZIMIDAZOLES AS STING ANTAGONISTS AND THE USE THEREOF AS MEDICAMENT

(71) Applicant: Boehringer Ingelheim International GmbH, Ingelheim am Rhein (DE)

(72) Inventors: Matthias HOFFMANN, Mittelbiberach (DE); Georg DAHMANN, Biberach an der Riss (DE); Patrick GROSS, Biberach an der Riss (DE); Jun LI, Danbury, CT (US); Herbert NAR, Ochsenhausen (DE); Thorsten OOST, Wien (AT)

(21) Appl. No.: 19/191,094

(22) Filed: Apr. 28, 2025

Related U.S. Application Data
(60) Provisional application No. 63/640,354, filed on Apr. 30, 2024.

(51) Int. Cl. C07D 403/14; A61K 31/4178; A61K 31/675; C07F 9/6558

(52) U.S. Cl. CPC C07D 403/14; A61K 31/4178; A61K 31/675; C07F 9/65583

(57) ABSTRACT

This invention relates to compounds of formula (I)

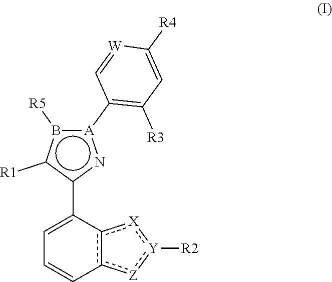

and their use in the prevention, delaying and/or treatment of diseases or conditions which can be influenced by STING inhibition.

This application claims priority to the U.S. provisional application 63/640,354, filed on Apr. 30, 2024, which is hereby incorporated by reference in its entirety.

### FIELD OF THE INVENTION

This invention relates to compounds of formula (I) and their use as STING antagonists e.g. for the treatment of a disease selected from the group consisting of systemic lupus erythematosus (SLE), cutaneous lupus, (monogenic and digenic) interferonopathies (including STING-associated vasculopathy with onset in infancy (SAVI), Aicardi-Goutieres syndrome (AGS), COPA syndrome, and familial chilblain lupus), type 1 interferonopathies with mutations in DNASE2 or ATAD3A genes, age-related macular degeneration (AMD), retinopathy, glaucoma, amyotrophic lateral sclerosis (ALS), Huntington disease, Alzheimer's disease, diabetes, obesity, inflammatory bowel disease (IBD), chronic obstructive pulmonary disease (COPD), Bloom's syndrome, Niemann-Pick Disease, Type C, ischaemic stroke, myotonic dystrophy type 2, Sjogren's syndrome, Parkinson's disease, heart failure, cancer, systemic sclerosis (SSc), vitiligo, prurigo nodularis, idiopathic inflammatory myopathy, myositis including dermatomyositis, metabolic dysfunction-associated steatotic liver disease (MASLD) (previously referred to as non-alcoholic fatty liver disease (NAFLD), metabolic dysfunction associated steatohepatitis (MASH, previously non-alcoholic steatotic hepatitis (NASH)), compensated and decompensated liver cirrhosis, acute on chronic liver failure (ACLF), alcoholic liver disease (ALD), interstitial lung disease (ILD), idiopathic pulmonary fibrosis (IPF), long COVID, aging/muscle disorders, sepsis, heart failure, anti-neutrophil cytoplasm antibody (ANCA) associated vasculitis, alopecia, chronic kidney disease, rheumatoid arthritis and osteoarthritis.

### BACKGROUND OF THE INVENTION

Innate immunity is considered a first line cellular stress response defending the host cell against invading pathogens and initiating signaling to the adaptive immune system. These processes are triggered by conserved pathogen-associated molecular patterns (PAMPs) through sensing by diverse pattern recognition receptors (PRRs) and subsequent activation of cytokine and type I interferon gene expression. The major antigen-presenting cells, such as monocytes, macrophages, and dendritic cells produce type I interferons and are critical for eliciting adaptive T- and B-cell immune system responses. The major PRRs detect aberrant, i.e. mislocalized, immature or unmodified nucleic acids on either the cell surface, the inside of lysosomal membranes or within other cellular compartments (Barbalat et al., Annu. Rev. Immunol. 29, 185-214 (2011)).

“Cyclic GMP-AMP Synthase” (cGAS) is the predominant sensor for aberrant double-stranded DNA (dsDNA) originating from pathogens or mislocalization or misprocessing of nuclear or mitochondrial cellular dsDNA (Sun et al., Science 339, 786-791 (2013); Wu et al., Science 339, 826-830 (2013); Ablasser et al., Nature 498, 380-384 (2013)). Binding of dsDNA to cGAS activates the reaction of GTP and ATP to form the cyclic dinucleotide GMP-AMP (referred to as cGAMP). cGAMP then binds to and activates the endoplasmatic reticulum membrane-anchored adaptor protein, “Stimulator of Interferon Genes” (STING, UniProtKB—Q86WV6). Activated STING recruits and activates TANK-binding kinase 1 (TBK1) which in turn phosporylates the transcription factor family of interferon regulatory factors (IRFs) inducing cytokine and type I interferon mRNA expression. STING activation by cGAMP also leads to activation of NF-kB signaling pathway and downstream production of proinflammatory cytokines (Sun et al., Science 339, 786-791 (2013). Human GoF STING mutants lead to an autoinflammatory syndrome, cutaneous vasculopathy and lung fibrosis (STING-associated vasculopathy with onset in infancy, SAVI). SAVI patients have a highly activated PBMCs and dermal fibroblasts, exhibiting an upregulated type-1 IFN signature and expression of NFκB-mediated profibrotic and proinflammatory genes (e.g. TNFα, IL-6) (Liu et al., 2014).

The critical role of STING in dsDNA sensing has been established in different pathogenic bacteria and viruses. Additionally, STING is essential in various other biological processes such as cellular senescence (Yang et al., PNAS 114, E4612 (2017), Glueck et al., Nat. Cell Biol. 19, 1061-1070 (2017)), autophagy and recognition of ruptured micronuclei in the surveillance of potential cancer cells (Mackenzie et al., Nature 548, 461-465 (2017); Harding et al., Nature 548, 466-470 (2017)).

While the cGAS/STING pathway is important for host defense against invading pathogens, cellular stress and genetic factors may also cause production of aberrant cellular dsDNA, e.g. by nuclear or mitochondrial leakage, and thereby trigger autoinflammatory responses. Aicardi-Goutieres syndrome (AGS; Crow et al., Nat. Genet. 38, 917-920 (2006))—a lupus-like severe autoinflammatory immune-mediated disorder—arises from genetic mutations such as loss-of-function mutations in TREX1, a primary DNA exonuclease responsible for degrading aberrant DNA in cytosol. Knock-out of STING in TREX1-deficient mice prevented otherwise lethal autoimmune responses, supporting STING as driver of interferonopathies (Gall et al., Immunity 36(1), 120-131 (2012); Gao et al., PNAS 112, E5699-E5705 (2015)). Likewise, embryonic lethality caused by deficiency of DNAse2, an endonuclease responsible for degradation of excessive DNA in lysosomes during endocytosis, was completely rescued by additional knock-out of STING (Ahn et al., PNAS 109, 19386-19391 (2012)).

A STING inhibitor may provide a therapeutic strategy for preventing (monogenic and digenic) interferonopathy diseases such as SAVI, AGS, familial chilblain lupus and COPA. A STING inhibitor will block inflammation and aberrant tissue remodeling in a cluster of autoimmune and inflammatory diseases including systemic lupus erythematosus (SLE), systemic sclerosis, vitiligo, prurigo nodularis, idiopathic inflammatory myopathy, myositis including dermatomyositis, inflammatory bowel disease, sepsis, Sjogren's syndrome, atopic dermatitis, as well as a cluster fibrosis diseases including NASH, IPF, chronic kidney fibrosis. A STING inhibitor also has applications to additional diseases such as cancer, heart failure, AMD, retinopathy, glaucoma, aging, decompensated liver cirrhosis, anti-neutrophil cytoplasm antibody (ANCA) associated vasculitis, alopecia, chronic kidney disease; Niemann-Pick Disease, Type C, ischaemic stroke, myotonic dystrophy type 2, Huntington disease, Bloom syndrome, Huntington disease, muscle disorders, rheumatoid arthritis, osteoarthritis, ALS, Parkinson's disease, Alzheimer's disease, COVID-19 (Decout et al, Nat Rev Immunol. 2021 21:548-569).

Due to the observation that inhibition of the STING pathway may provide a therapeutic strategy for preventing autoinflammation and for treating e.g. autoimmune diseases efforts to develop STING inhibitors or inhibition of the STING signaling pathway have been undertaken.
In WO2019122202 for example, compounds C-178 or C-176 are described interfering with STING signaling pathway in HEK293T cells or bone marrow derived macrophages (BMDMs) stimulated with a cyclic dinucleotide such as e.g. cGAMP which are irreversible inhibitors blocking the palmitoylation of STING at an allosteric site of STING.
In ACS Med Chem Lett. (2019, 10, 1, pp 92-97), Siu et all described novel cGAMP competitive ligands. It is believed that inhibiting the orthosteric site of cGAMP mediated STING activation leads to a suppression of all STING mediated activation in contrast to palmitoylation inhibitors. Compound 13 or 15 in this publication inhibits the HAQ STING variant (displacement assay) with a moderate IC50 of 84 or 41 nM and shows low cellular inhibitory activity of about 11 uM based on a cGAMP stimulated INFb production in THP1 cells.
In International patent application WO2019069270, claims modulators of STING which either activate or inhibit STING accordingly.
In the international patent applications WO23148129 and WO23237457 modulators of STING are disclosed that are relatively large macrocycles with demanding synthesis and handling of the molecules.

However, inhibitors of the STING receptor for therapeutic use face challenges. For example, it is expected that most inhibitors of the STING receptor binding its ligand binding site similar to the natural ligand, i.e. two molecules in the binding pocket. Yet, for the design of inhibitors of STING receptors this provides the additional challenge that the inhibitor molecules not only need to interact with the correct portion the STING receptor, but also will interact with the second molecule of the inhibitor in the ligand binding pocket of STING. Hence the potential interface between inhibitor and inhibitor is also important to consider for good inhibition results of STING.

Also, the polarity of the inhibitor molecules needs to be optimized on the one hand to allow sufficient crossing of the cell membranes to reach the target, while not enhancing the degradation of the inhibitor.

Another challenge for a therapeutic inhibitor of STING receptors is that in many STING mediated disease patients are likely to be co-administered with more than one medications to treat the symptoms of said diseases or the diseases itself. The inhibitors of STING should in such a situation not add additional workload to the detoxifying processes or catabolism of the other medication administered, which could lead to undesired changes in the half-life of any of the therapeutic compounds or have negative effects on the patient's metabolism.

### Aim of the Invention

It has now been found that compounds of the present invention according to general formula (I), or pharmaceutically acceptable salt thereof, are effective STING inhibitors.

In addition to the antagonistic property toward STING, the compounds of the present invention provide further advantageous properties as to be viable for human therapy, such as but not limited to: Being optimised for binding of two molecules of the inhibitor to the target's ligand binding pocket, sufficiently easy to synthesize and handle, good bioavailability, good mobility across the cell membrane and good access to the target receptor in the cells, acceptable cytotoxicity and/or genotoxicity, good ligand efficiency, good metabolic stability, low interaction with catabolic processes e.g. by cytochrome p450s or other CYP that are important with respect to possibly co-administered drugs, low degradation by light, e.g. sun light, yet good degradation ex-situ of the inhibitor or its break-down product e.g. in sewage plants.

The inhibitors of the invention perform better in one or several of these properties than the inhibitors of the STING receptor available so far. Accordingly, one aspect of the invention refers to compounds according to formula (I), or salts thereof as inhibitors of STING.

Another aspect of the invention refers to compounds according to formula (I), or salts thereof as inhibitors of STING optimised for binding of two molecules of the inhibitor to the target's ligand binding pocket and/or good ligand efficiency.

Another aspect of the invention refers to compounds according to formula (I), or salts thereof as inhibitors of STING having good metabolic stability and potency.

Another aspect of the invention refers to compounds according to formula (I), or salts thereof as inhibitors of STING optimised in polarity for good mobility across the cell membrane and good access to the target receptor in the cells while having good metabolic stability and potency.

Another aspect of the invention refers to compounds according to formula (I), or salts thereof as inhibitors of STING having good metabolic stability with acceptable cytotoxicity and/or genotoxicity.

Another aspect of the invention refers to compounds according to formula (I), or salts thereof as inhibitors of STING having good metabolic stability and low interaction with catabolic processes e.g. by cytochrome p450s or other CYP that are important with respect to possibly co-administered drugs.

Another aspect of the invention refers to compounds according to formula (I), or salts thereof as inhibitors of STING having good metabolic stability and low interaction with catabolic processes of other pharmaceutical compound administered overlappingly or simultaneously, including but not limited to further inhibitors of STING, and with acceptable cytotoxicity and/or genotoxicity.

Another aspect of the invention refers to compounds according to formula (I), or salts thereof as inhibitors of STING having good metabolic stability and low interaction with catabolic processes of other pharmaceutical compounds administered overlappingly or simultaneously, including but not limited to further inhibitors of STING, and with acceptable cytotoxicity and/or genotoxicity and optimised in polarity for good mobility across the cell membrane and good access to the target receptor in the cells and good potency.

Another aspect of the invention refers to compounds according to formula (I), or salts thereof as inhibitors of having good metabolic stability and low interaction with catabolic processes of other pharmaceutical compounds administered overlappingly or simultaneously, including but not limited to further inhibitors of STING, and with acceptable cytotoxicity and/or genotoxicity and optimised in polarity for good mobility across the cell membrane and good access to the target receptor in the cells and good potency and optimised for binding of two molecules of the inhibitor to the target's ligand binding pocket and good ligand efficiency.

In a further aspect this invention relates to pharmaceutical compositions containing at least one compound according to general formula (I), or pharmaceutically acceptable salts thereof, optionally together with one or more inert adjuvant, diluent and/or carrier.

A further aspect of the present invention relates to compounds according to general formula (I) or pharmaceutically acceptable salts thereof, or pharmaceutical compositions comprising compounds according to formula (I) or pharmaceutically acceptable salts thereof, for the use in the prevention of and/or treatment of and/or delaying the occurrence of and/or delaying the progression of disorders related to elevated and/or deregulated STING activity. In one aspect of the invention the use is to prevent one or more disorders related to elevated STING activity. Another aspect of the invention the use is to treat one or more disorders related to elevated STING activity. A further aspect the inventive use is to delay the occurrence of one or more disorders related to elevated STING activity.

In yet another aspect the inventive compounds and use is to delay the progression one or more disorders related to elevated STING activity, for example but not limited to progression of scleroderma renal crisis (SRC) to end stage renal disease/kidney failure; progression of MAFLD or MASH for example from MAFLD to MASH, or from MASH to Mash with cirrhosis as assessed with the NAFLD Activity Score (NAS) with or without steatosis, activity, and fibrosis (SAF) score and/or progression of Rheumatoid arthritis as assessed via the 2010 ACR/EULAR Rheumatoid Arthritis Classification Criteria for example but not limited to from a point value from 3 to 5 or from a point value 4 to point value 7.

Another aspect of the invention relates to processes of manufacture of the compounds of the present invention according to general formula (I) or salts thereof, particularly pharmaceutically acceptable salts.

Other aims of the present invention will become apparent to the skilled man directly from the foregoing and following remarks.

### DETAILED DESCRIPTION

In a first aspect the present invention relates to compounds of general formula (I)

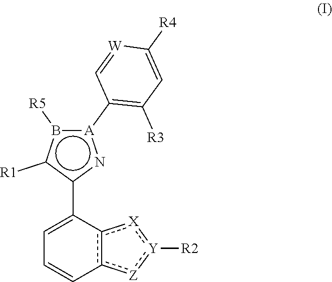

wherein
B-A is selected from the group B-A^{a} consisting of ═C—N— or —N—C═; this means A is C or N; B is C or N; but A and B are not N at the same time;
X—Y—Z is selected from the group X—Y—Z^{a} consisting of ═CH—N—N═, —N═C—NH— and —CH₂—N—C(O)—;
W is selected from the group W^{a} consisting of ═CH— and ═N—;
R¹ is selected from the group R1^{a} consisting of R6-C₁₋₅-alkyl- and C₃₋₆-cycloalkyl-;
R² is selected from the group R^{2a} consisting of;

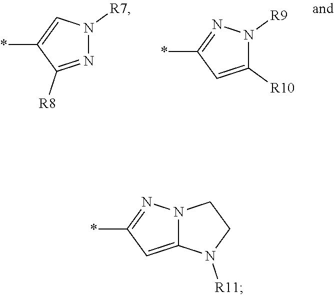

wherein * denotes the attachment point R² in formula (I)
R³ is selected from the group R^{3a} consisting of halogen, HO—, C₁₋₃-alkyl-, C₁₋₅-alkyl-O—, C₁₋₃-alkyl-O— C₁₋₃-alkyl-, C₃-s-alkenyl-O—, C₂₋₃-alkenyl-O—C₁₋₃-alkyl-C₃₋₆-cycloalkyl- and heterocyclyl-;
   wherein a C₁₋₃-alkyl-group is optionally substituted with 1 to 3 selected from the group consisting of fluorine, HO—, H₃C—O—, F₃C—O—, and F₂HC—O—;
   wherein the C₁₋₅-alkyl-group of the C₁₋₅-alkyl-O-group is optionally substituted with 1 to 5 (e.g. 2, 3 or 4) substituents independently selected from the group consisting of fluorine, HO—, H₂N—C(O)—, C₃₋₄-cycloalkyl-, C₁₋₃-alkyl-O—, heterocyclyl and heteroaryl;
R⁴ is selected from the group R^{4a} consisting of C₁₋₃-alkyl-S(O)₂—, C₁₋₃-alkyl-S(O)—, (C₁₋₃-alkyl)₂P(O)—, (C₁₋₃-alkyl)(C₃₋₆-cycloalkyl)P(O)—, —C(O)—, H₂N—C(O)—, C₁₋₃-alkyl-NH—C(O)—, (C₁₋₃-alkyl)₂N—C(O)— and

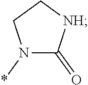 [SMILES: *N1CCNC1=O]

wherein * denotes the attachment point of R^{4a}
R⁵ is selected from the group R⁵¹ consisting of C₁₋₄-alkyl-;
R⁶ is selected from the group R⁶, consisting of H—, HO—and Halogen;
R⁷ is selected from the group R⁷¹ consisting of H—, Halogen, HO—, HO(C₁₋₆-alkyl)₂C—CH₂—, C₁₋₃-alkyl-O—, C₁₋₆-alkyl-, C₃-s-alkenyl-, C₃₋₆-cycloalkyl-, aryl, heteroaryl and heterocyclyl;
   wherein the C₁₋₆-alkyl-group is optionally substituted with 1 to 3 substituents independently selected from the group consisting of Halogen, HO—, and C₁₋₃-alkyl-O—,
   wherein the heteroaryl group is optionally substituted with 1 substituent selected from the group consisting of C₁₋₃-alkyl-,
   wherein the C₃₋₆-cycloalkyl- and/or aryl group is optionally substituted with 1 to 3 substituents independently selected from the group consisting of (H₃C)₂N—C(O)—;
R⁸ is selected from the group R^{8a} consisting of H—, C₁₋₆-alkyl-O—and heterocyclyl-O—;
   wherein the C₁₋₆-alkyl-O-group is optionally substituted with 1 substituent selected from the group consisting of C₁₋₃-alkyl-O—and (H₃C)₂N—C(O)—;
   C₁₋₅-alkyl-O—and Halogen;
R⁹ is selected from the group R^{9a} consisting of H—, C₁₋₃-alkyl- and H₂N—C(O)—CH₂—;
R¹⁰ is selected from the group R^{10 a} consisting of C₁₋₃-alkyl-, C₂₋₃-alkenyl-, C₁₋₃-alkyl-O—, C₁₋₃-alkyl-S—and C₃₋₄-cycloalkyl-,
   wherein the C₁₋₃-alkyl-group is optionally substituted with 1 to 3 substituents selected from the group consisting of F—;
R¹¹ is selected from the group R^{11a} consisting of C₁₋₅-alkyl-, or —C₁₋₅-alkyl-C(O)—, wherein the C₁₋₅-alkyl-, group is optionally substituted independently of one another with 1 to 3 substituents selected from the group consisting of C₁₋₃-alkyl-, halogen and HO—.
or a salt thereof, preferably a pharmaceutically acceptable salt.

Unless otherwise stated, the groups, residues, and substituents, particularly B-A, W, X—Y—Z, R¹, R², R³, R⁴, R⁵, R⁶, R⁷, R⁸, R⁹, R¹⁰ and R¹¹ are defined as above and hereinafter. If residues, substituents, or groups occur several times in a compound they may have the same or different meanings. Some preferred meanings of groups and substituents of the compounds according to the invention will be given hereinafter.

In a further embodiment of the present invention B-A is selected from the group B-A^{b} consisting of ═C—N—; this means A is N; B is C.

In a further embodiment of the present invention B-A is selected from the group B-A^{c} consisting of —N—C═; this means A is C; B is N.

In a further embodiment of the present invention X—Y—Z is selected from the group X—Y—Z^{b} consisting of ═CH—N—N═ and —N═C—NH—.

In a further embodiment of the present invention X—Y—Z is selected from the group X—Y—Z consisting of ═CH—N—N═.

In a further embodiment of the present invention X—Y—Z is selected from the group X—Y—Z^{d} consisting of —N═C—NH—.

In a further embodiment of the present invention W is selected from the group W^{b} consisting of ═CH—.

In a preferred embodiment, the compound is a compound of formula (Ia)

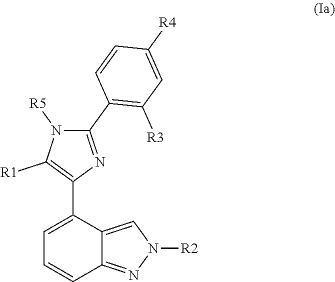

In a further embodiment of the present invention
R¹ is selected from the group R^{1b} consisting of isopropyl- and cyclopropyl-;

In a further embodiment of the present invention
R¹ is selected from the group R^{1c} consisting of cyclopropyl-;

In a further embodiment of the present invention
R² is selected from the group R^{2b} consisting of

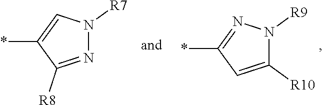

wherein * denotes the attachment points.

In a further embodiment of the present invention
R² is selected from the group R^{2c} consisting of

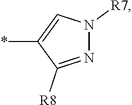

wherein * denotes the attachment point.

In a further embodiment of the present invention
R² is selected from the group R^{2d} consisting of

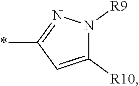

wherein * denotes the attachment point.

In a further embodiment of the present invention
R³ is selected from the group R^{3b} consisting of halogen, HO—, C₁₋₃-alkyl-, C₁-s-alkyl-O—, C₁₋₃-alkyl-O—C₁₋₃-alkyl-, C₃-s-alkenyl-O—, cyclopropyl- and heterocyclyl-;
   wherein the C₁₋₅-alkyl-group of the C₁₋₅-alkyl-O-group is optionally substituted with 1 to 3 substituents independently selected from the group consisting of fluorine, HO—, H₂N—C(O)—, C₃₋₄-cycloalkyl-, C₁₋₃-alkyl-O—, heterocyclyl and heteroaryl.

In a further embodiment of the present invention
R³ is selected from the group R^{3c} consisting of F—, Cl—, HO—, H₃C—, F₃C—, (H₃C)₂C—, H₃C—CH₂—, cyclopropyl-, H₃C—O—, FH₂C—O—, F₂HC—O—, F₃C—O—, (H₃C)₂CH—O—, (H₃C)₃C—O—, H₃C—CH₂—O—, F₂HC—CH₂—O—, F₂C(CH₃)—CH₂—O—, H₃C—CH₂—CH₂—O—, F—CH₂—CH₂—O—, HO—CH₂—CH₂—O—, H₂N—CH₂—C(O)—, H₂C═CH—CH₂—O—, H₃C—O—CH₂—CH₂—O—, H₃C—O—CH₂—CH₂—CH₂—O—, (H₃C)₂C(OH)—CH₂—CH₂—O—, (H₃C)₂CH—CH₂—O—, cyclopropyl-O—, cyclopropyl-CH₂—O—, H₃C—O—CH₂—CH₂—, H₃C—O—CH₂—,

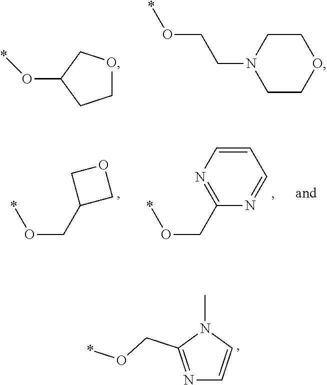

wherein * denotes the attachment point.

In a further embodiment of the present invention
R³ is selected from the group R^{3d} consisting of F—, Cl—, HO—, H₃C—, F₃C—, (H₃C)₂C—, H₃C—CH₂—, cyclopropyl-, H₃C—O—, FH₂C—O—, F₂HC—O—, F₃C—O—, (H₃C)₂CH—O—, (H₃C)₃C—O—, H₃C—CH₂—O—, F₂HC—CH₂—O—, F₂C(CH₃)—CH₂—O—, H₃C—CH₂—CH₂—O—, F—CH₂—CH₂—O—, HO—CH₂—CH₂—O—, H₂N—CH₂—C(O)—, H₂C═CH—CH₂—O—, H₃C—O—CH₂—CH₂—O—, H₃C—O—CH₂—CH₂—CH₂—O—, (H₃C)₂C(OH)—CH₂—CH₂—O—, (H₃C)₂CH—CH₂—O—, H₃C—O—CH₂—, cyclopropyl-O—, cyclopropyl-CH₂—O—.

In a further embodiment of the present invention
R³ is selected from the group R^{3e} consisting of H₃C—, F₂HC—CH₂—O—, F₃C—O—, and H₃C—O—CH₂—.

In a further embodiment of the present invention
R³ is selected from the group R^{3f} consisting of H₃C—.

In a further embodiment of the present invention
R³ is selected from the group R^{3g} consisting of F₂HC—CH₂—O—.

In a further embodiment of the present invention
R³ is selected from the group R^{3h} consisting of F₃C—O—.

In a further embodiment of the present invention
R³ is selected from the group R³ⁱ consisting of H₃C—O—CH₂—.

In a further embodiment of the present invention
R⁴ is selected from the group R^{4b} consisting of (C₁₋₃-alkyl)₂P(O)—, C₁₋₃-alkylNH—C(O)—, (C₁₋3-alkyl)₂N—C(O)— and H₂N—C(O)—.

In a further embodiment of the present invention
R⁴ is selected from the group R⁴ consisting of (CH₃CH₂)₂P(O)—, (CH₃)(CH₃CH₂)P(O)—, (CH₃)₂P(O)—, (CH₃)NH—C(O)—, (CH₃)₂N—C(O)— and H₂N—C(O)—.

In a further embodiment of the present invention
R⁴ is selected from the group R^{4d} consisting of (CH₃CH₂)₂P(O)—, (CH₃)(CH₃CH₂)P(O)—and (CH₃)₂P(O)—.

In a further embodiment of the present invention
R⁴ is selected from the group R⁴¹ consisting of (CH₃CH₂)₂P(O)—.

In a further embodiment of the present invention
R⁴ is selected from the group R⁴⁰ consisting of (CH₃)(CH₃CH₂)P(O)—.

In a further embodiment of the present invention
R⁴ is selected from the group R⁴⁹ consisting of (CH₃)₂P(O)—.

In a further embodiment of the present invention
R⁴ is selected from the group R^{4h} consisting of H₂N—C(O)—.

In a further embodiment of the present invention
R⁵ is selected from the group R^{5b} consisting of H₃C—, H₃C—CH₂—, H₃C—CH₂—CH₂—and (H₃C)₂C—.

In a further embodiment of the present invention
R⁵ is selected from the group R^{5C} consisting of H₃C—.

In a further embodiment of the present invention
R⁵ is selected from the group R^{5d} consisting of H₃C—CH₂—.

In a further embodiment of the present invention
R⁶ is selected from the group R^{6b} consisting of H—, HO—, F—and Cl—.

In a further embodiment of the present invention
R⁶ is selected from the group R^{6c} consisting of H—and HO—.

In a further embodiment of the present invention
R⁶ is selected from the group R6^{d} consisting of H—.

In a further embodiment of the present invention
R⁷ is selected from the group R^{7b} consisting of C₁₋₃-alkyl-, C₃₋₄-cycloalkyl- and HO(C₁₋₃-alkyl)₂C—CH₂—.

In a further embodiment of the present invention
R⁷ is selected from the group R^{7c} consisting of H₃C—, cyclopropyl-, HO(CH₃)₂C—CH₂—.

In a further embodiment of the present invention
R⁷ is selected from the group R^{7d} consisting of H₃C—.

In a further embodiment of the present invention
R⁷ is selected from the group R⁷¹ consisting of cyclopropyl-.

In a further embodiment of the present invention
R⁷ is selected from the group R^{7f} consisting of HO(CH₃)₂C—CH₂—.

In a further embodiment of the present invention
R⁸ is selected from the group R^{5b} consisting of C₁₋₅-alkyl-O.

In a further embodiment of the present invention
R⁸ is selected from the group R^{8C} consisting of C₁₋₃-alkyl-O—In a further embodiment of the present invention
R⁸ is selected from the group R^{8d} consisting of H₃C—O—.

In a further embodiment of the present invention
R⁸ is selected from the group R^{e} consisting of H₃C—CH₂—O—.

In a further embodiment of the present invention
R⁹ is selected from the group R^{9b} consisting of H—and H₃C—and H₃C—CH₂—.

In a further embodiment of the present invention
R⁹ is selected from the group R^{9c} consisting of H—.

In a further embodiment of the present invention
R⁹ is selected from the group R^{9d} consisting of H₃C—.

In a further embodiment of the present invention
R⁹ is selected from the group R^{9e} consisting of H₃C—CH₂—.

In a further embodiment of the present invention
R¹⁰ is selected from the group R^{10b} consisting of C₁₋₃-alkyl-, and C₃₋₄-cycloalkyl-,
   wherein the C₁₋₃-alkyl-group is optionally substituted with 1 to 3 substituents selected from the group consisting of F.

In a further embodiment of the present invention
R¹⁰ is selected from the group R^{10c} consisting of cyclopropyl-, F₂H C—and F₃C—.

In a further embodiment of the present invention
R¹⁰ is selected from the group R^{10d} consisting of cyclopropyl-.

In a further embodiment of the present invention
R¹⁰ is selected from the group R¹⁰ consisting of F₃C—.
R¹⁰ is selected from the group R^{10 f} consisting of F₂HC—.
R¹¹ is selected from the group R^{11b} consisting of C₁₋₅-alkyl-, wherein the C₁₋₅_alkyl-, group is optionally substituted with a substituent selected from the group consisting of C₁₋₃-alkyl-, fluorine or HO—.
R¹¹ is selected from the group R^{11c} consisting of F₂HC—, F₃C—, H₃C, H₃C—CH₂— and H₃C—CH₂—CH₂—.

B-A, W, X—Y—Z, R¹, R², R³, R⁴, R⁵, R⁶, R⁷, R⁸, R⁹, R¹⁰ and R¹¹ represents a characterized, individual embodiment for the corresponding substituent as described above. Thus, given the above definitions, individual embodiments of the first aspect of the invention are fully characterized by the term (B-A^{X}, W^{X}, X—Y—Z^{X}, R^{1X}, R^{2X}, R^{3X}, R^{4X}, R^{5X}, R^{6X}, R^{7X}, R^{8X}, R^{9X}, R^{10X} and R^{11X}), wherein for each index ‘x’ an individual figure is given that ranges from ‘a’ to the highest letter given above. All individual embodiments described by the term in parentheses with full permutation of the indices ‘x’, referring to the definitions above, shall be comprised by the present invention.

The following table 1 shows such embodiments E-1 to E-20 of the compound of general formula (I) or a salt thereof, preferably a pharmaceutically acceptable salt, that are considered preferred.

TABLE 1
Embodiments E-1 to E-20 of the invention

| | B-A^{x} | W^{X} | X-Y-Z^{X} | R^{1x} | R^{2x} | R^{3x} | R^{4x} | R^{5x} | R^{6x} | R^{7x} | R^{8x} | R^{9x} | R^{10x} | R^{11x} |
|---|---|---|---|---|---|---|---|---|---|---|---|---|---|---|
| E-1 | B-A^{a} | W^{a} | X-Y-Z^{a} | R^{1a} | R^{2a} | R^{3a} | R^{4a} | R^{5a} | R^{6a} | R^{7a} | R^{8a} | R^{9a} | R^{10a} | R^{11a} |
| E-2 | B-A^{a} | W^{a} | X-Y-Z^{a} | R^{1a} | R^{2a} | R^{3a} | R^{4a} | R^{5a} | R^{6b} | R^{7a} | R^{8a} | R^{9a} | R^{10a} | R^{11a} |
| E-3 | B-A^{a} | W^{a} | X-Y-Z^{a} | R^{1a} | R^{2a} | R^{3a} | R^{4a} | R^{5a} | R^{6c} | R^{7a} | R^{8a} | R^{9a} | R^{10a} | R^{11a} |
| E-4 | B-A^{a} | W^{a} | X-Y-Z^{a} | R^{1a} | R^{2a} | R^{3a} | R^{4a} | R^{5a} | R^{6d} | R^{7a} | R^{8a} | R^{9a} | R^{10a} | R^{11a} |
| E-5 | B-A^{a} | W^{c} | X-Y-Z^{a} | R^{1a} | R^{2a} | R^{3a} | R^{4a} | R^{5a} | R^{6a} | R^{7a} | R^{8a} | R^{9a} | R^{10a} | R^{11a} |
| E-6 | B-A^{a} | W^{b} | X-Y-Z^{a} | R^{1a} | R^{2a} | R^{3a} | R^{4a} | R^{5a} | R^{6a} | R^{7a} | R^{8a} | R^{9a} | R^{10a} | R^{11a} |
| E-7 | B-A^{a} | W^{b} | X-Y-Z^{a} | R^{1b} | R^{2a} | R^{3a} | R^{4a} | R^{5a} | — | R^{7a} | R^{8a} | R^{9a} | R^{10a} | R^{11a} |
| E-8 | B-A^{a} | W^{b} | X-Y-Z^{a} | R^{1b} | R^{2a} | R^{3b} | R^{4a} | R^{5a} | — | R^{7a} | R^{8a} | R^{9a} | R^{10a} | R^{11a} |
| E-9 | B-A^{b} | W^{b} | X-Y-Z^{a} | R^{1b} | R^{2a} | R^{3b} | R^{4a} | R^{5a} | — | R^{7a} | R^{8a} | R^{9a} | R^{10a} | R^{11a} |
| E-10 | B-A^{c} | W^{b} | X-Y-Z^{a} | R^{1b} | R^{2a} | R^{3b} | R^{4a} | R^{5a} | — | R^{7a} | R^{8a} | R^{9a} | R^{10a} | R^{11a} |
| E-11 | B-A^{c} | W^{b} | X-Y-Z^{b} | R^{1b} | R^{2a} | R^{3b} | R^{4a} | R^{5a} | — | R^{7a} | R^{8a} | R^{9a} | R^{10a} | R^{11a} |
| E-12 | B-A^{c} | W^{b} | X-Y-Z^{b} | R^{1b} | R^{2a} | R^{3c} | R^{4a} | R^{5a} | — | R^{7a} | R^{8a} | R^{9a} | R^{10a} | R^{11a} |
| E-13 | B-A^{c} | W^{b} | X-Y-Z^{b} | R^{1b} | R^{2a} | R^{3c} | R^{4a} | R^{5b} | — | R^{7a} | R^{8a} | R^{9a} | R^{10a} | R^{11a} |
| E-14 | B-A^{c} | W^{b} | X-Y-Z^{b} | R^{1b} | R^{2a} | R^{3c} | R^{4b} | R^{5b} | — | R^{7a} | R^{8a} | R^{9a} | R^{10a} | R^{11a} |
| E-15 | B-A^{c} | W^{b} | X-Y-Z^{b} | R^{1b} | R^{2b} | R^{3c} | R^{4b} | R^{5b} | — | R^{7a} | R^{8a} | R^{9a} | R^{10a} | — |
| E-16 | B-A^{c} | W^{b} | X-Y-Z^{b} | R^{1b} | R^{2b} | R^{3d} | R^{4b} | R^{5b} | — | R^{7a} | R^{8a} | R^{9a} | R^{10a} | — |
| E-17 | B-A^{c} | W^{b} | X-Y-Z^{b} | R^{1b} | R^{2b} | R^{3d} | R^{4b} | R^{5b} | — | R^{7a} | R^{8a} | R^{9a} | R^{10b} | — |
| E-18 | B-A^{c} | W^{b} | X-Y-Z^{b} | R^{1b} | R^{2c} | R^{3d} | R^{4c} | R^{5b} | — | R^{7c} | R^{8c} | — | — | — |
| E-19 | B-A^{c} | W^{b} | X-Y-Z^{b} | R^{1b} | R^{2d} | R^{3e} | R^{4c} | R^{5b} | — | — | — | R^{9b} | R^{10b} | — |
| E-20 | B-A^{c} | W^{b} | X-Y-Z^{c} | R^{1c} | R^{2d} | R^{3e} | R^{4d} | R^{5b} | — | — | — | R^{9d} | R^{10c} | — |
| E-21 | B-A^{c} | W^{b} | X-Y-Z^{c} | R^{1c} | R^{2d} | R^{3h} | R^{4f} | R^{5c} | — | — | — | R^{9c} | R^{10e} | — |
| E-22 | B-A^{c} | W^{b} | X-Y-Z^{c} | R^{1c} | R^{2d} | R^{3h} | R^{4g} | R^{5c} | — | — | — | R^{9c} | R^{10e} | — |
| E-23 | B-A^{c} | W^{b} | X-Y-Z^{d} | R^{1c} | R^{2d} | R^{3h} | R^{4f} | R^{5c} | — | — | — | R^{9c} | R^{10e} | — |
| E-24 | B-A^{c} | W^{b} | X-Y-Z^{c} | R^{1c} | R^{2d} | R^{3h} | R^{4g} | R^{5c} | — | — | — | R^{9c} | R^{10f} | — |
| E-25 | B-A^{c} | W^{b} | X-Y-Z^{c} | R^{1c} | R^{2d} | R^{3h} | R^{4f} | R^{5c} | — | — | — | R^{9c} | R^{10f} | — |
| E-26 | B-A^{c} | W^{b} | X-Y-Z^{c} | R^{1c} | R^{2d} | R^{3h} | R^{4g} | R^{5c} | — | — | — | R^{9c} | R^{10e} | — |
| E-27 | B-A^{c} | W^{b} | X-Y-Z^{c} | R^{1c} | R^{2d} | R^{3h} | R^{4f} | R^{5c} | — | — | — | R^{9c} | R^{10e} | — |
| E-28 | B-A^{c} | W^{b} | X-Y-Z^{c} | R^{1c} | R^{2d} | R^{3h} | R^{4f} | R^{5c} | — | — | — | R^{9c} | R^{10d} | — |
| E-29 | B-A^{c} | W^{b} | X-Y-Z^{c} | R^{1c} | R^{2d} | R^{3h} | R^{4f} | R^{5c} | — | — | — | R^{9d} | R^{10e} | — |
| E-30 | B-A^{c} | W^{b} | X-Y-Z^{c} | R^{1c} | R^{2d} | R^{3h} | R^{4g} | R^{5c} | — | — | — | R^{9d} | R^{10e} | — |
| E-31 | B-A^{c} | W^{b} | X-Y-Z^{d} | R^{1c} | R^{2d} | R^{3h} | R^{4f} | R^{5c} | — | — | — | R^{9d} | R^{10e} | — |
| E-32 | B-A^{c} | W^{b} | X-Y-Z^{c} | R^{1c} | R^{2d} | R^{3h} | R^{4g} | R^{5c} | — | — | — | R^{9d} | R^{10f} | — |
| E-33 | B-A^{c} | W^{b} | X-Y-Z^{c} | R^{1c} | R^{2d} | R^{3h} | R^{4f} | R^{5c} | — | — | — | R^{9d} | R^{10f} | — |
| E-35 | B-A^{c} | W^{b} | X-Y-Z^{c} | R^{1c} | R^{2d} | R^{3h} | R^{4h} | R^{5c} | — | — | — | R^{9d} | R^{10e} | — |
| E-36 | B-A^{c} | W^{b} | X-Y-Z^{c} | R^{1c} | R^{2d} | R^{3h} | R^{4f} | R^{5c} | — | — | — | R^{9d} | R^{10e} | — |
| E-37 | B-A^{c} | W^{b} | X-Y-Z^{c} | R^{1c} | R^{2d} | R^{3h} | R^{4f} | R^{5c} | — | — | — | R^{9d} | R^{10d} | — |

Accordingly, for example E-14 covers compounds of general formula (I),

wherein
B-A is selected from the group B-A^{c} consisting of —N—C═; this means A is C; B is N;
W is selected from the group W^{b} consisting of ═CH—;
X—Y—Z is selected from the group X—Y—Z^{b} consisting of ═CH—N—N═ and —N═C—NH—;
R¹ is selected from the group R^{1b} consisting of isopropyl- and cyclopropyl-;
R² is selected from the group R², consisting of
   R² is selected from the group R², consisting of

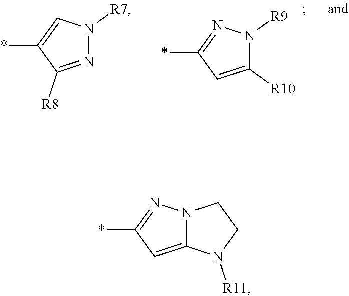

wherein * denotes the attachment point to the core;
R³ is selected from the group R^{3c} consisting of F—, Cl—, HO—, H₃C—, F₃C—, (H₃C)₂C—, H₃C—CH₂—, cyclopropyl-, H₃C—O—, FH₂C—O—, F₂HC—O—, F₃C—O—, (H₃C)₂CH—O—, (H₃C)₃C—O—, H₃C—CH₂—O—, F₂HC—CH₂—O—, F₂C(CH₃)—CH₂—O—, H₃C—CH₂—CH₂—O—, F—CH₂—CH₂—O—, HO—CH₂—CH₂—O—, H₂N—CH₂—C(O)—, H₂C═CH—CH₂—O—, H₃C—O—CH₂—CH₂—O—, H₃C—O—CH₂—CH₂—CH₂—O—, (H₃C)₂C(OH)—CH₂—CH₂—O—, (H₃C)₂CH—CH₂—O—, cyclopropyl-O—, cyclopropyl-CH₂—O—, H₃C—O—CH₂—CH₂—, H₃C—O—CH₂—,

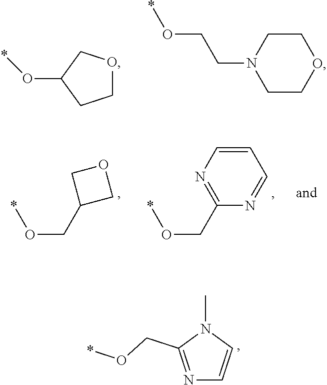

wherein * denotes the attachment point;
R⁴ is selected from the group R^{4b} consisting of (C₁₋₃-alkyl)₂P(O)—, C₁₋₃-alkylNH—C(O)—, (C₁₋₃-alkyl)₂N—C(O)— and H₂N—C(O)—;
R⁵ is selected from the group R^{5b} consisting of H₃C—, H₃C—CH₂—, H₃C—CH₂—CH₂—and (H₃C)₂C—;
R⁷ is selected from the group R⁷¹ consisting of H—, Halogen, HO—, HO(C₁₋₆-alkyl)₂C—CH₂—, C₁₋₃-alkyl-O—, C₁₋₆-alkyl-, C₃-s-alkenyl-, C₃₋₆-cycloalkyl-, aryl, heteroaryl and heterocyclyl;
   wherein the C₁₋₆-alkyl-group is optionally substituted with 1 to 3 substituents independently selected from the group consisting of Halogen, HO—, and C₁₋₃-alkyl-O—,
   wherein the heteroaryl group is optionally substituted with 1 substituent selected from the group consisting of C₁₋₃-alkyl-,
   wherein the C₃₋₆-cycloalkyl- and/or aryl group is optionally substituted with 1 to 3 substituents independently selected from the group consisting of (H₃C)₂N—C(O)—;
R⁸ is selected from the group R¹¹ consisting of H—, C₁₋₆-alkyl-O—and heterocyclyl-O—;
   wherein the C₁₋₆-alkyl-O-group is optionally substituted with 1 substituent selected from the group consisting of C₁₋₃-alkyl-O—and (H₃C)₂N—C(O)—;
   C₁₋₅-alkyl-O—and Halogen;
R⁹ is selected from the group R^{9a} consisting of H—, C₁₋₃-alkyl- and H₂N—C(O)—CH₂—;
R¹⁰ is selected from the group R^{10 a} consisting of C₁₋₃-alkyl-, C₂₋₃-alkenyl-, C₁₋₃-alkyl-O—, C₁₋₃-alkyl-S—and C₃₋₄-cycloalkyl-,
   wherein the C₁₋₃-alkyl-group is optionally substituted with 1 to 3 substituents selected from the group consisting of F—;
R¹¹ is selected from the group R^{11a} consisting of C₁₋₅-alkyl-, or —C₁₋₅-alkyl-C(O)—, wherein the C₁₋₅alkyl-, group is optionally substituted independently of one another with 1 to 3 substituents selected from the group consisting of C₁₋₃-alkyl-, halogen and HO—;
or a salt thereof, preferably a pharmaceutically acceptable salt.

Accordingly, for example E-18 covers compounds of general formula (I),

wherein
B-A is selected from the group B-A^{c} consisting of —N—C═; this means A is C; B is N;
W is selected from the group W^{b} consisting of ═CH—;
X—Y—Z is selected from the group X—Y—Z^{b} consisting of ═CH—N—N═ and —N═C—NH—;
R¹ is selected from the group R^{1b} consisting of isopropyl- and cyclopropyl-;
R² is selected from the group R^{2c} consisting of

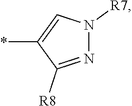

wherein * denotes the attachment point;
R³ is selected from the group R^{3d} consisting of F—, Cl—, HO—, H₃C—, F₃C—, (H₃C)₂C—, H₃C—CH₂—, cyclopropyl-, H₃C—O—, FH₂C—O—, F₂HC—O—, F₃C—O—, (H₃C)₂CH—O—, (H₃C)₃C—O—, H₃C—CH₂—O—, F₂HC—CH₂—O—, F₂C(CH₃)—CH₂—O—, H₃C—CH₂—CH₂—O—, F—CH₂—CH₂—O—, HO—CH₂—CH₂—O—, H₂N—CH₂—C(O)—, H₂C═CH—CH₂—O—, H₃C—O—CH₂—CH₂—O—, H₃C—O—CH₂—CH₂—CH₂—O—, (H₃C)₂C(OH)—CH₂—CH₂—O—, (H₃C)₂CH—CH₂—O—, H₃C—O—CH₂—, cyclopropyl-O—, cyclopropyl-CH₂—O—;
R⁴ is selected from the group R^{4c} consisting of (CH₃CH₂)₂P(O)—, (CH₃)(CH₃CH₂)P(O)—, (CH₃)₂P(O)—, (CH₃)NH—C(O)—, (CH₃)₂N—C(O)— and H₂N—C(O)—;
R⁵ is selected from the group R^{5b} consisting of H₃C—, H₃C—CH₂—, H₃C—CH₂—CH₂—and (H₃C)₂C—;
R⁷ is selected from the group R^{7c} consisting of H₃C—, cyclopropyl-, HO(CH₃)₂C—CH₂—;
R⁸ is selected from the group R^{8c} consisting of C₁₋₃-alkyl-O—;
or a salt thereof, preferably a pharmaceutically acceptable salt.

Accordingly, for example E-20 covers compounds of general formula (I),

wherein
B-A is selected from the group B-A^{c} consisting of —N—C═; this means A is C; B is N,
W is selected from the group W^{b} consisting of ═CH—;
X—Y—Z is selected from the group X—Y—Z^{c} consisting of ═CH—N—N═;
R¹ is selected from the group R^{1c} consisting of cyclopropyl-;
R² is selected from the group R^{2d} consisting of

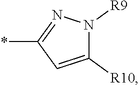

wherein * denotes the attachment point;
R³ is selected from the group R^{3e} consisting of H₃C—, F₂HC—CH₂—O—, F₃C—O—, and H₃C—O—CH₂—;
R⁴ is selected from the group R^{4d} consisting of (CH₃CH₂)₂P(O)—, (CH₃)(CH₃CH₂)P(O)—and (CH₃)₂P(O)—;
R⁵ is selected from the group R^{5b} consisting of H₃C—, H₃C—CH₂—, H₃C—CH₂—CH₂—and (H₃C)₂C—;
R⁹ is selected from the group R^{9d} consisting of H₃C—;
R¹⁰ is selected from the group R^{10c} consisting of cyclopropyl-CF₂H—and CF₃—;
or a salt thereof, preferably a pharmaceutically acceptable salt.

Further preferred are the following compounds listed in table 2 or table 2A or salt thereof or stereoisomers thereof (the No. refers to the No. assigned to the compound in the experimental section). Each compound of table 2 or table 2A is represented without indicating the stereochemistry thereof, if any. Specific information concerning stereochemical properties of compounds of table 2 or table 2A can be taken from the experimental section. In case the final compounds according of said experimental section are salt forms, they can be converted into the neutral compound by conventional methods.

TABLE 2
Examples I to XX

| No. | Structure |
|---|---|
| I | 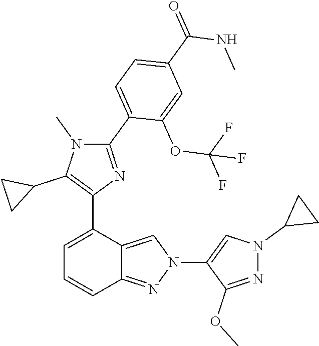 [SMILES: CNC(=O)c1ccc(-c2nc(-c3cccc4nn(-c5cn(C6CC6)nc5OC)cc34)c(C3CC3)n2C)c(OC(F)(F)F)c1] |
| II | 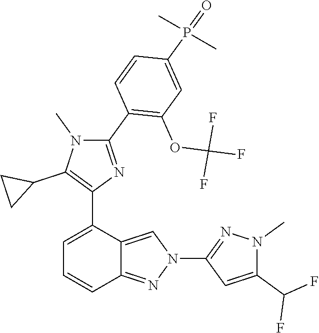 [SMILES: Cn1nc(-n2cc3c(-c4nc(-c5ccc(P(C)(C)=O)cc5OC(F)(F)F)n(C)c4C4CC4)cccc3n2)cc1C(F)F] |
| III | 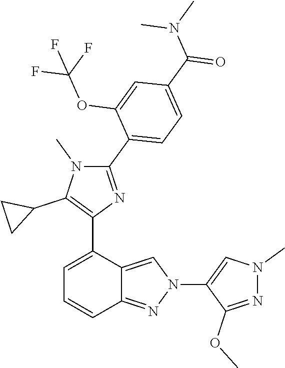 [SMILES: COc1nn(C)cc1-n1cc2c(-c3nc(-c4ccc(C(=O)N(C)C)cc4OC(F)(F)F)n(C)c3C3CC3)cccc2n1] |
| IV | 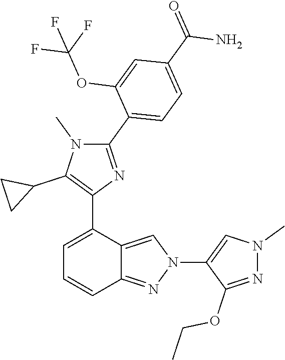 [SMILES: CCOc1nn(C)cc1-n1cc2c(-c3nc(-c4ccc(C(N)=O)cc4OC(F)(F)F)n(C)c3C3CC3)cccc2n1] |
| V | 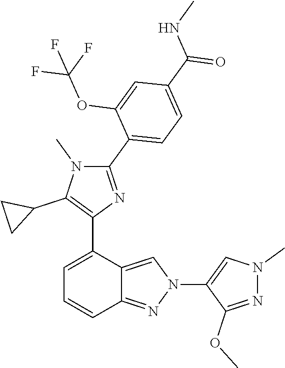 [SMILES: CNC(=O)c1ccc(-c2nc(-c3cccc4nn(-c5cn(C)nc5OC)cc34)c(C3CC3)n2C)c(OC(F)(F)F)c1] |
| VI | 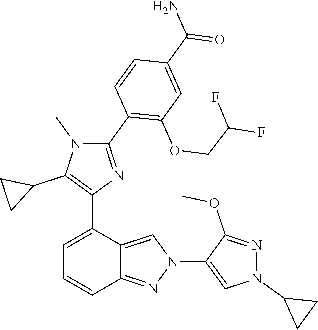 [SMILES: COc1nn(C2CC2)cc1-n1cc2c(-c3nc(-c4ccc(C(N)=O)cc4OCC(F)F)n(C)c3C3CC3)cccc2n1] |
| VII | 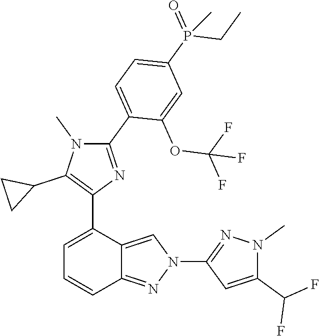 [SMILES: CCP(C)(=O)c1ccc(-c2nc(-c3cccc4nn(-c5cc(C(F)F)n(C)n5)cc34)c(C3CC3)n2C)c(OC(F)(F)F)c1] |
| VIII | 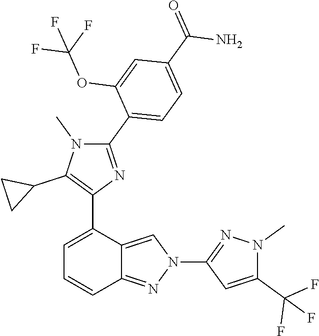 [SMILES: Cn1nc(-n2cc3c(-c4nc(-c5ccc(C(N)=O)cc5OC(F)(F)F)n(C)c4C4CC4)cccc3n2)cc1C(F)(F)F] |
| IX | 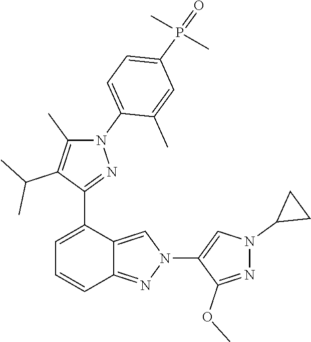 [SMILES: COc1nn(C2CC2)cc1-n1cc2c(-c3nn(-c4ccc(P(C)(C)=O)cc4C)c(C)c3C(C)C)cccc2n1] |
| X | 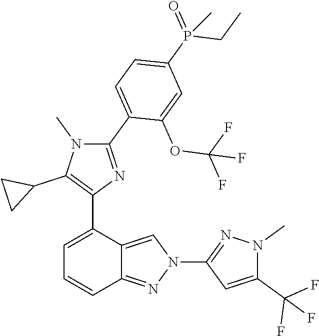 [SMILES: CCP(C)(=O)c1ccc(-c2nc(-c3cccc4nn(-c5cc(C(F)(F)F)n(C)n5)cc34)c(C3CC3)n2C)c(OC(F)(F)F)c1] |
| XI | 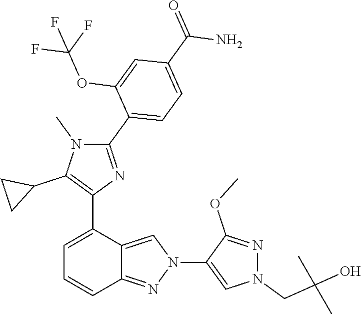 [SMILES: COc1nn(CC(C)(C)O)cc1-n1cc2c(-c3nc(-c4ccc(C(N)=O)cc4OC(F)(F)F)n(C)c3C3CC3)cccc2n1] |
| XII | 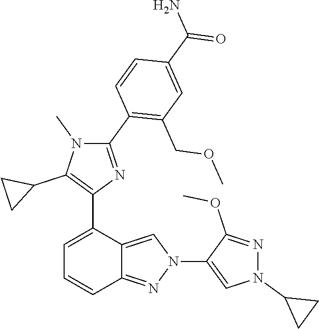 [SMILES: COCc1cc(C(N)=O)ccc1-c1nc(-c2cccc3nn(-c4cn(C5CC5)nc4OC)cc23)c(C2CC2)n1C] |
| XIII | 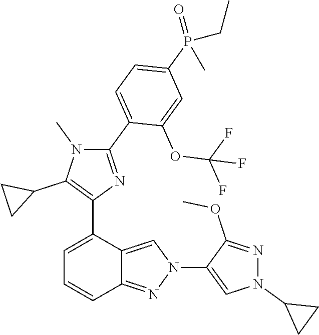 [SMILES: CCP(C)(=O)c1ccc(-c2nc(-c3cccc4nn(-c5cn(C6CC6)nc5OC)cc34)c(C3CC3)n2C)c(OC(F)(F)F)c1] |
| XIV | 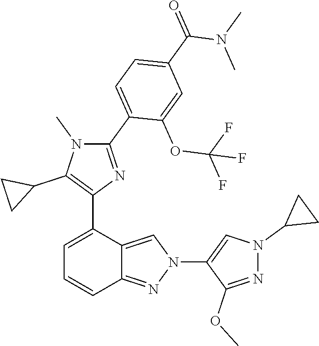 [SMILES: COc1nn(C2CC2)cc1-n1cc2c(-c3nc(-c4ccc(C(=O)N(C)C)cc4OC(F)(F)F)n(C)c3C3CC3)cccc2n1] |
| XV | 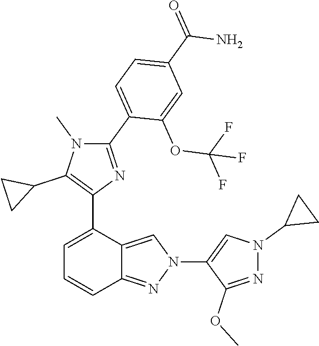 [SMILES: COc1nn(C2CC2)cc1-n1cc2c(-c3nc(-c4ccc(C(N)=O)cc4OC(F)(F)F)n(C)c3C3CC3)cccc2n1] |
| XVI | 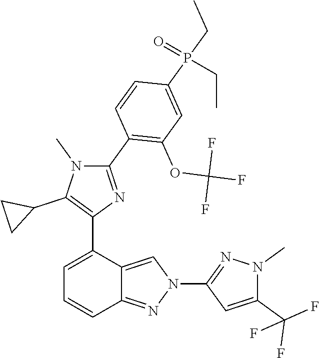 [SMILES: CCP(=O)(CC)c1ccc(-c2nc(-c3cccc4nn(-c5cc(C(F)(F)F)n(C)n5)cc34)c(C3CC3)n2C)c(OC(F)(F)F)c1] |
| XVII | 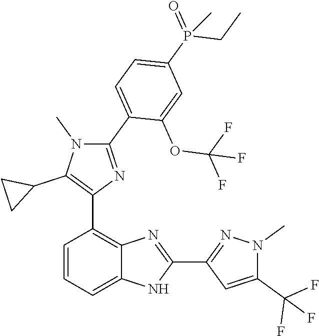 [SMILES: CCP(C)(=O)c1ccc(-c2nc(-c3cccc4[nH]c(-c5cc(C(F)(F)F)n(C)n5)nc34)c(C3CC3)n2C)c(OC(F)(F)F)c1] |
| XVIII | 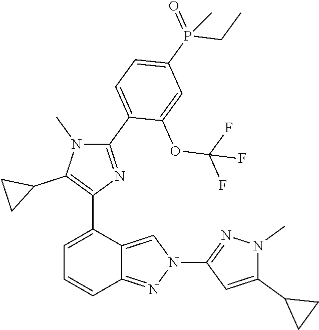 [SMILES: CCP(C)(=O)c1ccc(-c2nc(-c3cccc4nn(-c5cc(C6CC6)n(C)n5)cc34)c(C3CC3)n2C)c(OC(F)(F)F)c1] |
| XIX | 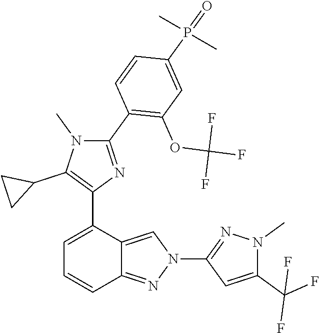 [SMILES: Cn1nc(-n2cc3c(-c4nc(-c5ccc(P(C)(C)=O)cc5OC(F)(F)F)n(C)c4C4CC4)cccc3n2)cc1C(F)(F)F] |
| XX | 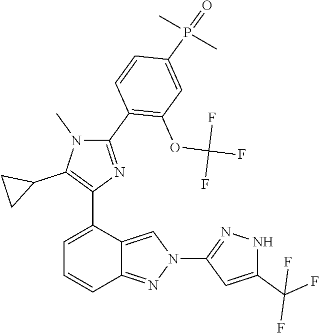 [SMILES: Cn1c(-c2ccc(P(C)(C)=O)cc2OC(F)(F)F)nc(-c2cccc3nn(-c4cc(C(F)(F)F)[nH]n4)cc23)c1C1CC1] |

In a preferred embodiment, the compounds of the invention are selected from one or more examples I to XX in Table 2, or salts thereof or stereoisomers thereof.

TABLE 2A
Examples XXI to XXX

| | |
|---|---|
| XXI | 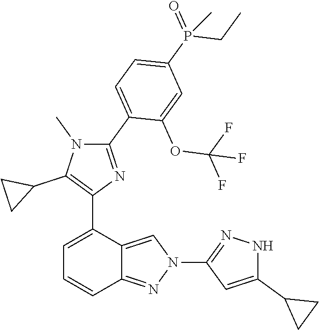 [SMILES: CCP(C)(=O)c1ccc(-c2nc(-c3cccc4nn(-c5cc(C6CC6)[nH]n5)cc34)c(C3CC3)n2C)c(OC(F)(F)F)c1] |
| XXII | 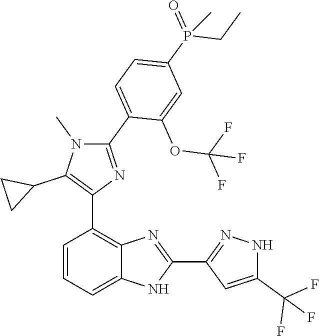 [SMILES: CCP(C)(=O)c1ccc(-c2nc(-c3cccc4[nH]c(-c5cc(C(F)(F)F)[nH]n5)nc34)c(C3CC3)n2C)c(OC(F)(F)F)c1] |
| XXIII | 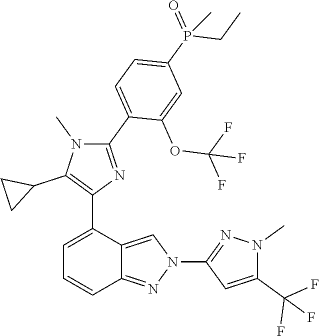 [SMILES: CCP(C)(=O)c1ccc(-c2nc(-c3cccc4nn(-c5cc(C(F)(F)F)n(C)n5)cc34)c(C3CC3)n2C)c(OC(F)(F)F)c1] |
| XXIV | 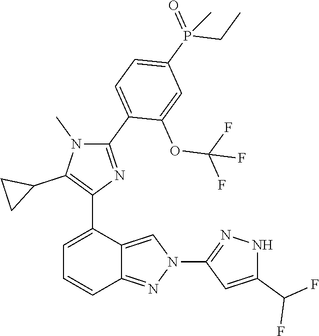 [SMILES: CCP(C)(=O)c1ccc(-c2nc(-c3cccc4nn(-c5cc(C(F)F)[nH]n5)cc34)c(C3CC3)n2C)c(OC(F)(F)F)c1] |
| XXV | 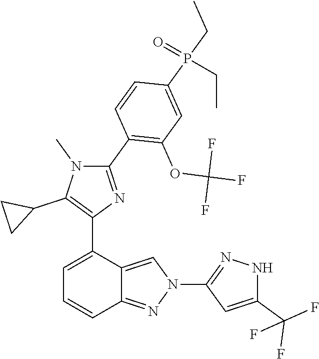 [SMILES: CCP(=O)(CC)c1ccc(-c2nc(-c3cccc4nn(-c5cc(C(F)(F)F)[nH]n5)cc34)c(C3CC3)n2C)c(OC(F)(F)F)c1] |
| XXVI | 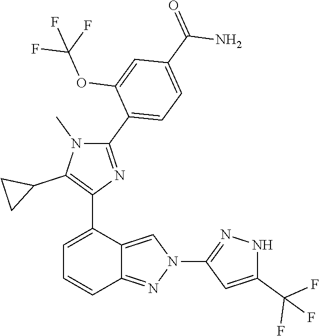 [SMILES: Cn1c(-c2ccc(C(N)=O)cc2OC(F)(F)F)nc(-c2cccc3nn(-c4cc(C(F)(F)F)[nH]n4)cc23)c1C1CC1] |
| XXVII | 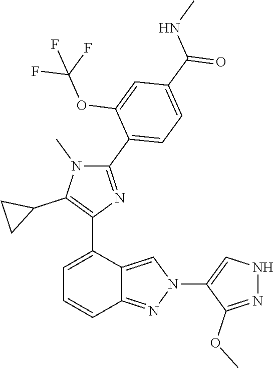 [SMILES: CNC(=O)c1ccc(-c2nc(-c3cccc4nn(-c5c[nH]nc5OC)cc34)c(C3CC3)n2C)c(OC(F)(F)F)c1] |
| XXVIII | 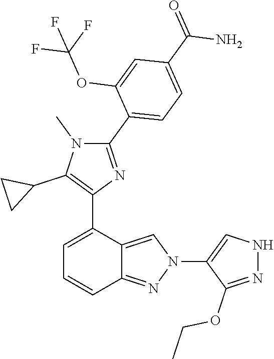 [SMILES: CCOc1n[nH]cc1-n1cc2c(-c3nc(-c4ccc(C(N)=O)cc4OC(F)(F)F)n(C)c3C3CC3)cccc2n1] |
| XXIX | 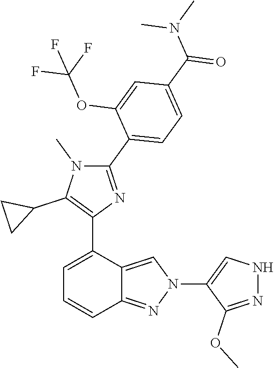 [SMILES: COc1n[nH]cc1-n1cc2c(-c3nc(-c4ccc(C(=O)N(C)C)cc4OC(F)(F)F)n(C)c3C3CC3)cccc2n1] |
| XXX | 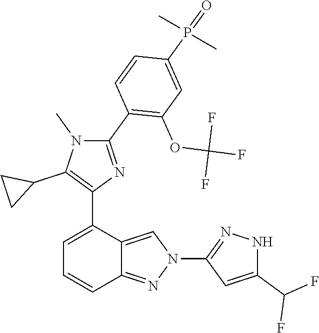 [SMILES: Cn1c(-c2ccc(P(C)(C)=O)cc2OC(F)(F)F)nc(-c2cccc3nn(-c4cc(C(F)F)[nH]n4)cc23)c1C1CC1] |

or a salt of any thereof.

A further embodiment of the present invention covers the compounds of general formula (I), preferably of formula (Ia), particularly the compounds listed in table 2 or table 2A, more preferably those listed in table 2, in form of their pharmaceutically acceptable salts.

A further embodiment of the present invention refers to pharmaceutical compositions comprising at least one compound according to formula (I), preferably according to formula (Ia), or pharmaceutically acceptable salts thereof, optionally together with at least one inert adjuvant, diluent and/or carrier.

In a further embodiment, the present invention relates to a compound of the present invention or a pharmaceutically acceptable salt thereof, or a pharmaceutical composition comprising at least one compound according to general formula (I), preferably according to formula (Ia), or pharmaceutically acceptable salts thereof, for use as a medicament.

In a further embodiment, the present invention relates to compounds according to general formula (I), preferably according to formula (Ia), or pharmaceutically acceptable salts thereof, or pharmaceutical compositions comprising compounds according to general formula (I), preferably according to formula (Ia), or pharmaceutically acceptable salts thereof, for use in the prevention, the delaying of the occurrence, the delaying of the progression and/or treatment of diseases or conditions which can be influenced by STING nhibition. Inhibition of the STING protein may not require to be a complete inhibition of the STING proteins within a cell, tissue, organ or the body of a patient to cause the desired positive effects in a patient. A partial inhibition maybe sufficient and possibly desirable in some patients.

### Used Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general, in groups like HO—, H₂N—, (O)S—, (O)₂S—, NC-(cyano), HOOC—, F₃C—or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkyl-” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail.

The numeration of the atoms of a substituent starts with the atom which is closest to the core or to the group to which the substituent is attached. For example, the term “3-carboxypropyl-group” represents the following substituent:

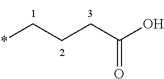 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

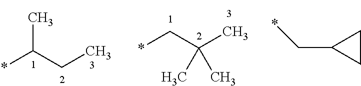

The asterisk may be used in sub-formulas to indicate the bond which is connected to the core molecule as defined.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and the appended claims, a given chemical formula or name shall encompass tautomer's and all stereo, optical and geometrical isomers (e.g. enantiomers, diastereomers, E/Z isomers etc.,) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts, or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

The term halogen denotes fluorine, chlorine, bromine and iodine.

The term “C₁₋ₙ-alkyl-”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₂₋ₘ-alkenyl” is used for a group “C₂₋ₘ-alkyl” wherein m is an integer selected from 3, 4, 5 or 6, preferably 4, 5 or 6, if at least two carbon atoms of said group are bonded to each other by a double bond.

The term “C₃₋ₖ-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

The term “carbocyclyl”, either alone or in combination with another radical, means a mono, bi or tricyclic ring structure consisting of 3 to 14 carbon atoms. The term “carbocyclyl” refers to fully saturated, partially saturated and aromatic ring systems. The term “carbocyclyl” encompasses fused, bridged and spirocyclic systems. Examples without limitation are:

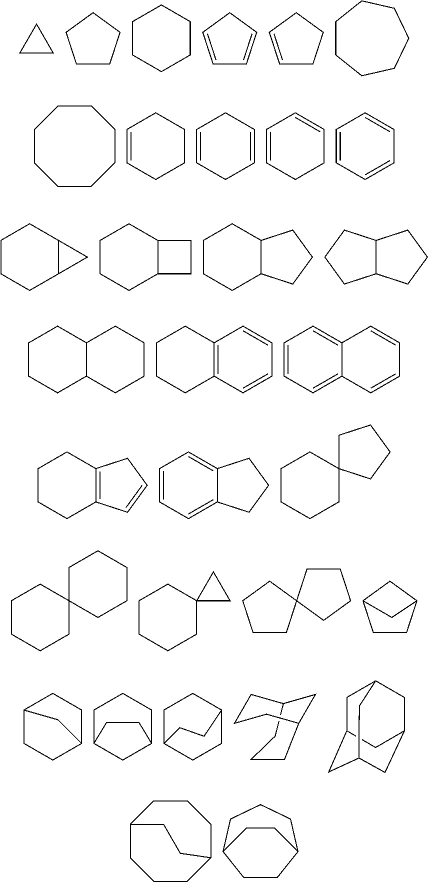

The term “aryl” as used herein, either alone or in combination with another radical, denotes a carbocyclic aromatic monocyclic group containing 6 carbon atoms which is optionally further fused to a second five or six membered, carbocyclic group which is aromatic, saturated or unsaturated. Aryl includes, but is not limited to, phenyl, indanyl, indenyl, naphthyl, anthracenyl, phenanthrenyl, tetrahydronaphthyl and dihydronaphthyl.

The term “heterocyclyl” means a saturated or unsaturated mono- or polycyclic ring system optionally comprising aromatic rings, containing one or more heteroatoms selected from N, O, S, SO or SO₂ consisting of 3 to 14 ring atoms wherein none of the heteroatoms is part of the aromatic ring. The term “heterocyclyl” is intended to include all the possible isomeric forms.

Thus, the term “heterocyclyl” includes the following exemplary structures (not depicted as radicals as each form is optionally attached through a covalent bond to any atom so long as appropriate valences are maintained):

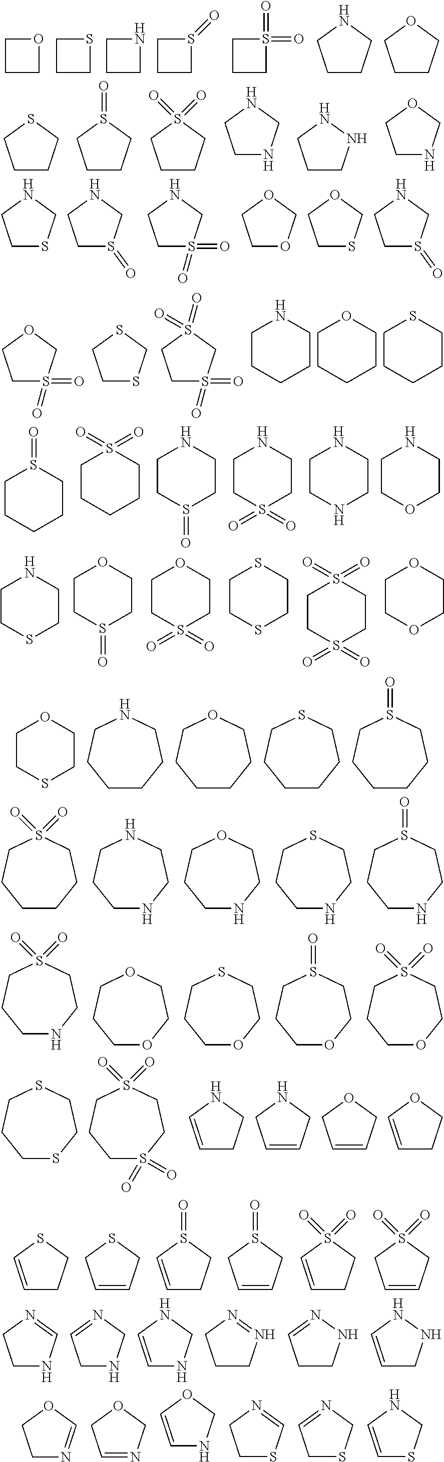

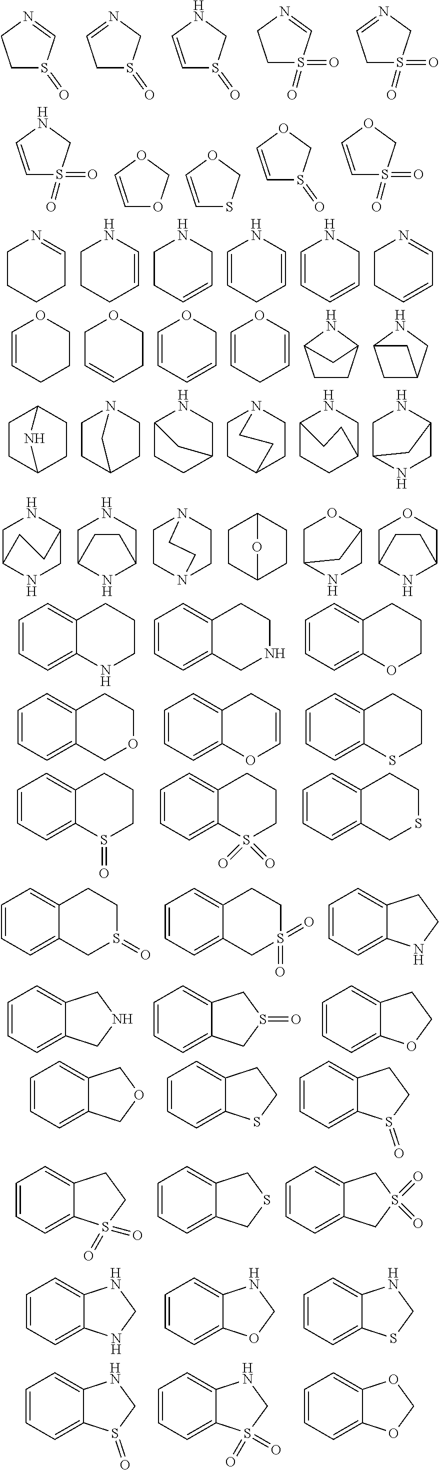

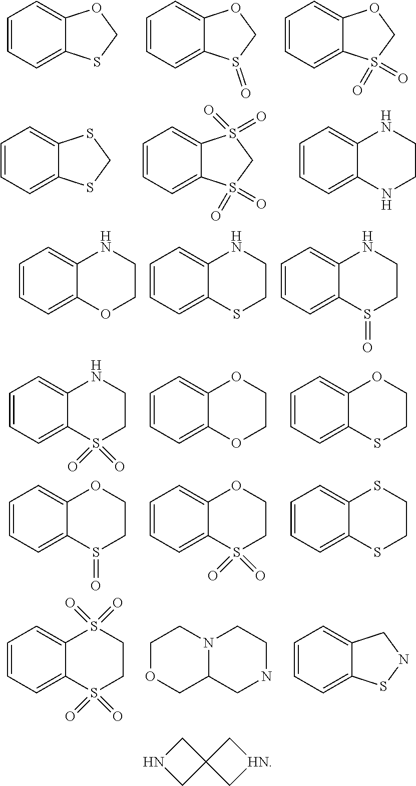

The term “heteroaryl” means a mono- or polycyclic ring system, comprising at least one aromatic ring, containing one or more heteroatoms selected from N, O, S, SO or SO₂, consisting of 5 to 14 ring atoms wherein at least one of the heteroatoms is part of an aromatic ring. The term “heteroaryl” is intended to include all the possible isomeric forms.

Thus, the term “heteroaryl” includes the following exemplary structures (not depicted as radicals as each form is optionally attached through a covalent bond to any atom so long as appropriate valences are maintained):

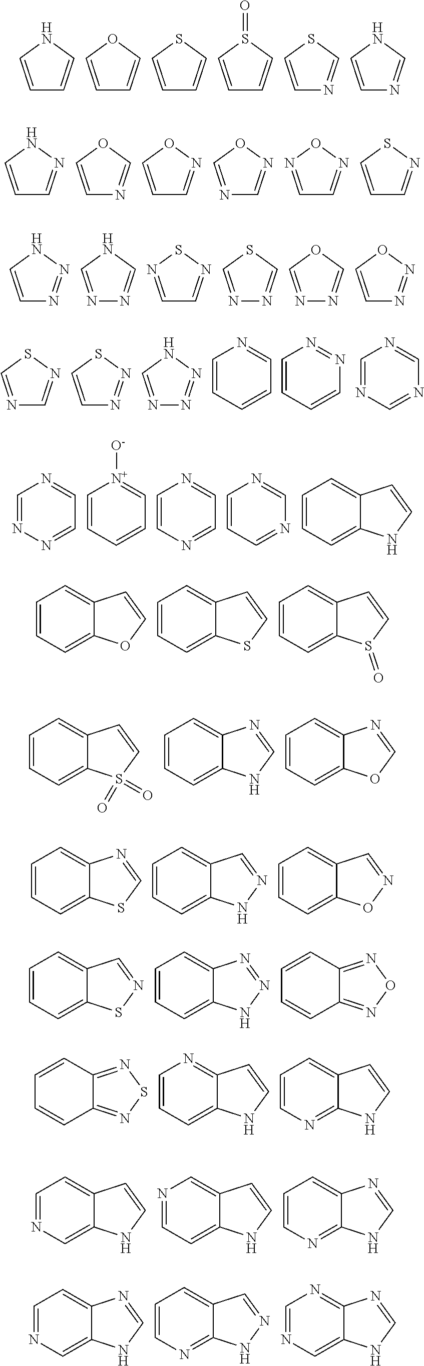

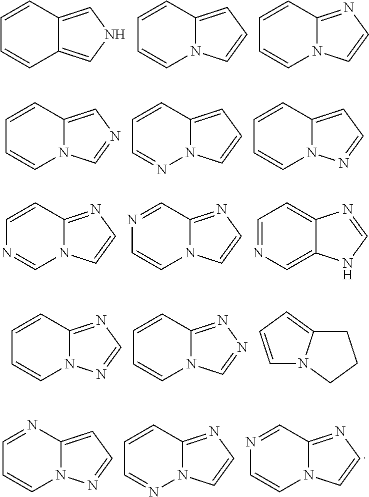

Many of the terms given above may be used repeatedly in the definition of a formula or group and in each case have one of the meanings given above, independently of one another.

The term “bicyclic ring systems” means groups consisting of 2 joined cyclic substructures including spirocyclic, fused, and bridged ring systems.

### Synthesis

The compounds according to the invention may be obtained using methods of synthesis known in principle, known to the one skilled in the art and described in the literature of organic synthesis. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used. Preferably, the compounds are obtained by the following methods according to the invention which are described in more detail hereinafter.

The following Schemes illustrate generally how to manufacture the compounds of the present invention by way of example. Starting materials may be prepared by methods that are described in the literature or herein or may be prepared in an analogous or similar manner. Any functional groups in the starting materials or intermediates may be protected using conventional protecting groups. These protecting groups may be cleaved again at a suitable stage within the reaction sequence using methods familiar to the one skilled in the art. The abbreviated substituents may be as defined above if not defined otherwise within the context of the schemes.

Optimum reaction conditions and reaction times may vary depending on reactants used. Unless otherwise specified, solvents, temperatures, pressures, and other reaction conditions may be readily selected by one of ordinary skill in the art. Specific procedures are provided in the Experimental section. Typically, reaction progress may be monitored by thin layer chromatography (TLC), liquid chromatography—mass spectrometry (LC-MS) if desired, and intermediates and products may be purified by chromatography and/or by recrystallization.

The examples which follow are illustrative and, as recognized by one skilled in the art, particular reagents or conditions could be modified as needed for individual compounds without undue experimentation. Starting materials and intermediates used, in the methods below, are either commercially available or easily prepared from commercially available materials by those skilled in the art.

### Examples and Experimental Data

The following examples are for the purpose of illustration of the invention only and are not intended in any way to limit the scope of the present invention.

The term “room temperature” designate a temperature of about 20° C., e.g., 15 to 25° C.

As a rule, ¹H-NMR and/or mass spectra have been obtained for the compounds prepared.

Flash chromatography or MPLC is performed with commercial silica gel and is equivalent to silica gel chromatography.

Absolute configuration of representative examples is either defined via the chemical starting material, single crystal x-ray structure determination or protein-ligand X-ray determinations.

Unless otherwise specified, compounds containing chiral centers have the stereochemistry depicted. The assignment of stereochemistry has been made either by use of a chiral starting material of known stereochemistry, by stereoselective synthesis of known stereochemistry, or by biological activity.

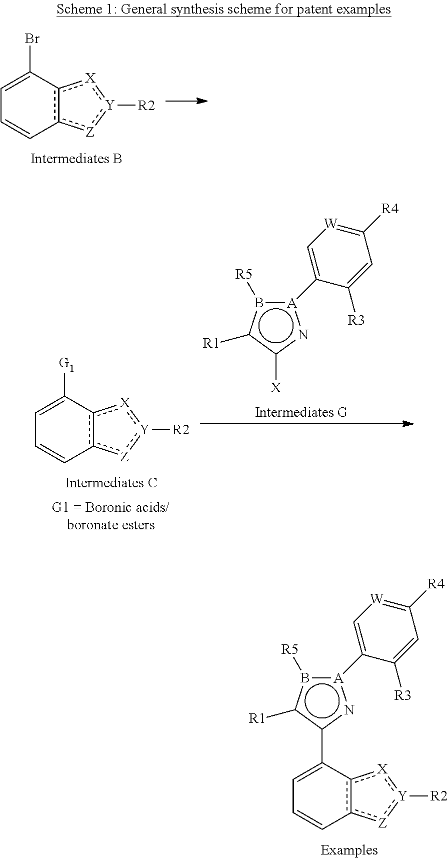

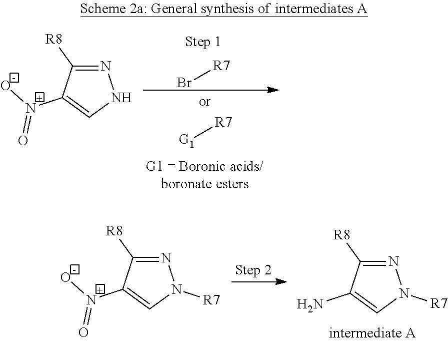

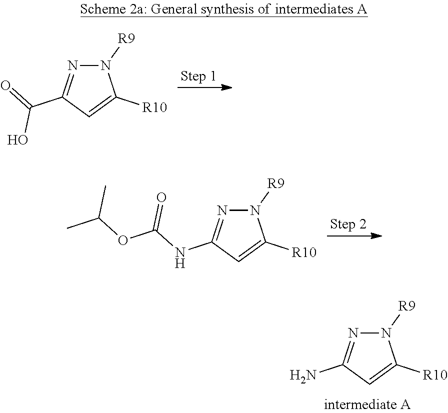

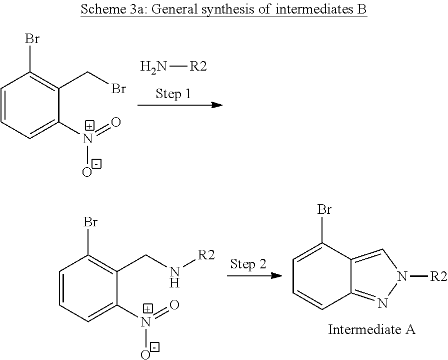

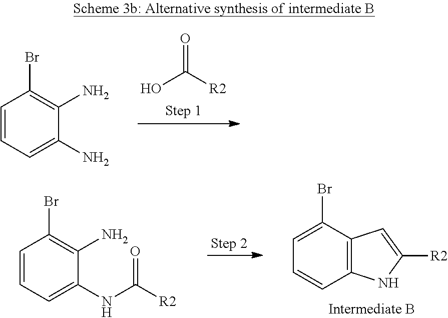

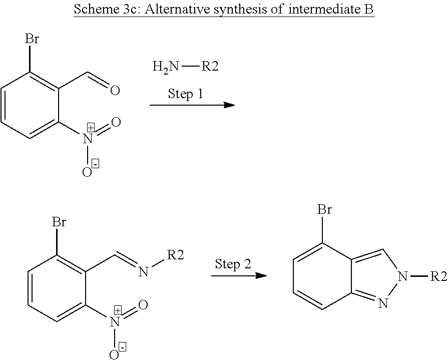

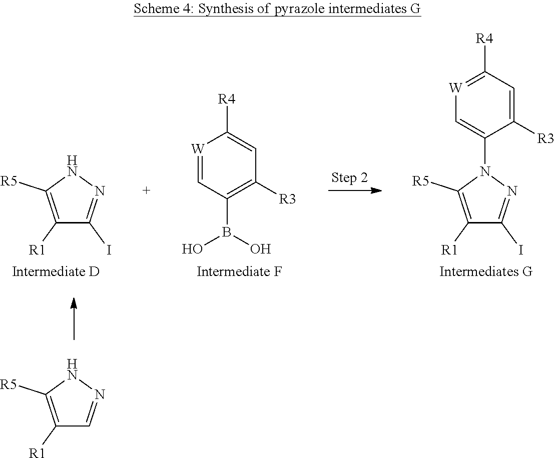

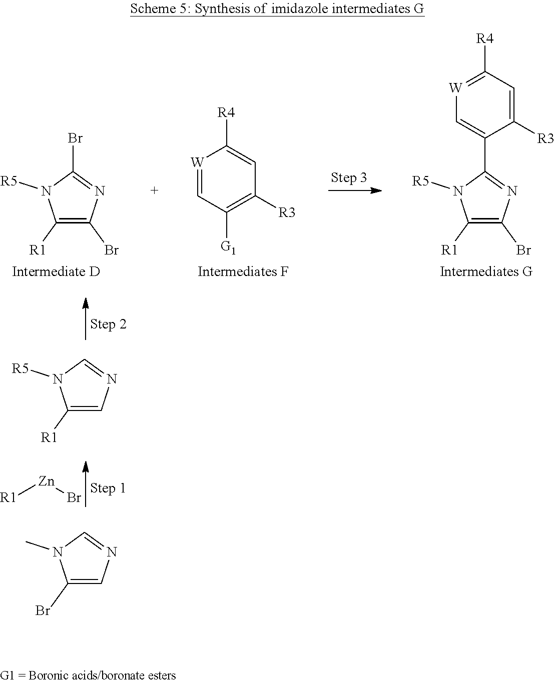

All starting materials not described are either commercially available or described in literature. Absolute stereochemistry was either determined by single Xray analysis or by Xray of protein ligand complexes of examples.

### Synthesis of Intermediates A1-A4

### Synthesis of Intermediate A1

### Step 1: Synthesis of 1-cyclopropyl-3-methoxy-4-nitro-1H-pyrazole

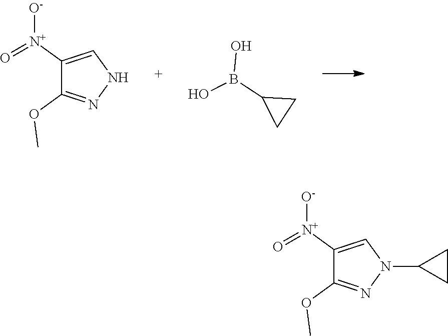

3-Methoxy-4-nitro-1H-pyrazole (4.00 g, 28.0 mmol) and cyclopropylboronic acid (4.32 g, 50.3 mmol) are dissolved in ACN (80 mL) and DCM (20 mL). Copper (II) acetate (12.7 g, 69.9 mmol) and pyridine (12.0 mL) are added, and the reaction mixture is stirred under air at 65° C. for 5 d. The reaction mixture is filtered through a pad of Celite and the filtrate is evaporated. The residue is purified by reversed phase chromatography (HPLC; ACN/water/TFA) to afford the desired compound.

Analysis (method A): Rₜ: 0.38 min, [M+H]⁺: 184

### Step 2: Synthesis of 1-cyclopropyl-3-methoxy-1H-pyrazol-4-amine (Intermediate A1)

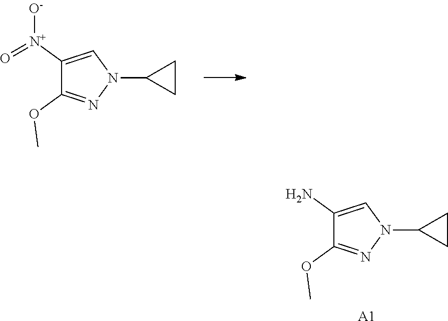

1-Cyclopropyl-3-methoxy-4-nitro-1H-pyrazole (2.00 g, 10.9 mmol) is dissolved in MeOH (60 mL). Pd/C 10% (200 mg) is added, and the reaction mixture is hydrogenated at RT and 3 bar for 1 h. The reaction mixture is filtered, 4 M HCl in dioxane (15 mL) is added and the filtrate is evaporated to afford the intermediate A1.

Analysis (method A): Rₜ: 0.20 min, [M+H]⁺: 154

### Synthesis of Intermediate A2

### Step 1: Synthesis of 4-(3-methoxy-4-nitro-1H-pyrazol-1-yl)-2-methylbutan-2-ol

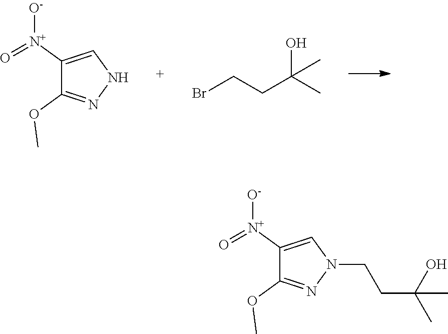

3-Methoxy-4-nitro-1H-pyrazole (600 mg, 4.19 mmol) is dissolved in DMF (10 mL). K₂CO₃ (2.89 g, 21.0 mmol) and 4-bromo-2-methylbutan-2-ol (1.40 g, 8.39 mmol) are added, and the reaction mixture is stirred at RT for 2 h. The reaction is purified by reversed phase chromatography (HPLC; ACN/water/NH₃) to afford the desired compound.

Analysis (method B): Rₜ: 0.76 min, [M+H]⁺: 230

### Step 2: Synthesis of 4-(4-amino-3-methoxy-1H-pyrazol-1-yl)-2-methylbutan-2-ol (Intermediate A2)

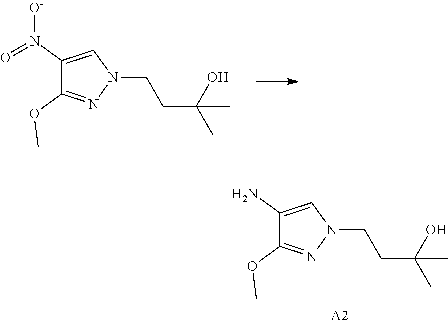 [SMILES: COc1nn(CCC(C)(C)O)cc1[N+](=O)[O-] ; COc1nn(CCC(C)(C)O)cc1N ; "A2"]

4-(3-Methoxy-4-nitro-1H-pyrazol-1-yl)-2-methylbutan-2-ol (900 mg, 3.93 mmol) is dissolved in MeOH (30 mL). Pd/C 10% (100 mg) is added, and the reaction mixture is hydrogenated at RT and 50 psi for 2 h. The reaction mixture is filtered, and the filtrate is evaporated to afford the intermediate A2.

Analysis (method B): Rₜ: 0.59 min, [M+H]⁺: 200

### Synthesis of Intermediate A3

### Step 1: Synthesis of tert-butyl N-[5-(difluoromethyl)-1-methyl-1H-pyrazol-3-yl]carbamate

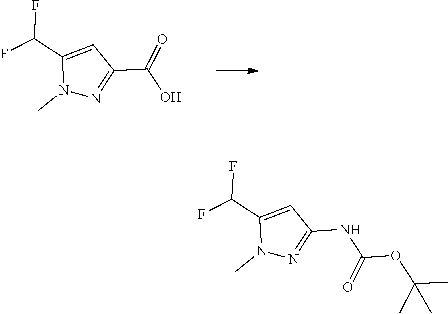 [SMILES: Cn1nc(C(=O)O)cc1C(F)F ; Cn1nc(NC(=O)OC(C)(C)C)cc1C(F)F]

5-(Difluoromethyl)-1-methyl-1H-pyrazole-3-carboxylic acid (1.00 g, 5.39 mmol) is dissolved in t-BuOH (15 mL) and TEA (2.30 mL, 16.4 mmol). Diphenylphosphoryl azide (1.40 mL, 6.49 mmol) is slowly added dropwise over a period of 10 min, and the reaction mixture is stirred at RT for 15 min and at 80° C. for 3 h and again at RT overnight. The reaction mixture is concentrated, and the crude residue is purified by reversed phase chromatography (HPLC; ACN/water/NH₃) to afford the desired compound.

Analysis (method C): Rₜ: 0.56 min, [M+H-isobuten]⁺: 192

### Step 2: Synthesis of 5-(difluoromethyl)-1-methyl-1H-pyrazol-3-amine (Intermediate A3)

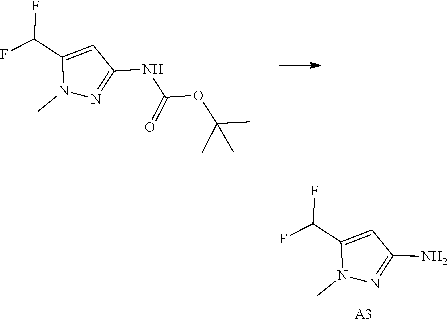

Tert-butyl N-[5-(difluoromethyl)-1-methyl-1H-pyrazol-3-yl]carbamate (485 mg, 1.96 mmol) is dissolved in dioxane (4 mL). HCl in dioxane (4 M, 6.00 mL, 24.0 mmol) is added, and the reaction mixture is stirred at RT for 18 h. The reaction mixture is concentrated, triturated with DCM (10 mL) and evaporated to dryness to afford the intermediate A3.

Analysis (method D): Rₜ: 0.21 min, [M+H]⁺: 148

### Synthesis of Intermediate A4

### Step 1: Synthesis of 1-(3-methoxy-4-nitro-1H-pyrazol-1-yl)-2-methylpropan-2-ol

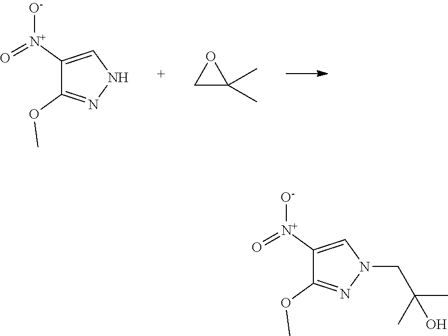

In a microwave vial 3-methoxy-4-nitro-1H-pyrazole (1.00 g, 6.64 mmol) is dissolved in DMF (20 mL). Cs₂CO₃ (4.33 g, 13.3 mmol) and 2,2-dimethyloxirane (1.78 mL, 19.9 mmol) are added, and the reaction mixture is stirred in the closed vial at 100° C. for 2 h. The reaction is purified by reversed phase chromatography (HPLC; ACN/water/NH₃) to afford the desired compound.

Analysis (method B): Rₜ: 0.71 min, [M+H]⁺: 216

### Step 2: Synthesis of 1-(4-amino-3-methoxy-1H-pyrazol-1-yl)-2-methylpropan-2-ol (Intermediate A4)

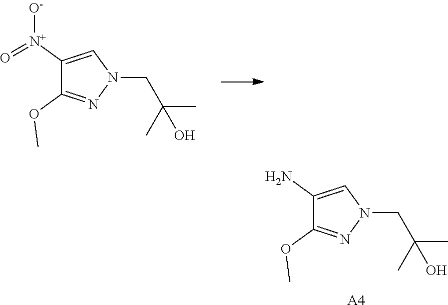

1-(3-Methoxy-4-nitro-1H-pyrazol-1-yl)-2-methylpropan-2-ol (350 mg, 1.63 mmol) is dissolved in MeOH (20 mL). Pd/C 10% (50 mg) is added, and the reaction mixture is hydrogenated at RT and 3 bar for 2 h. The reaction mixture is filtered, and the filtrate is evaporated to afford the intermediate A4.

Analysis (method B): Rₜ: 0.44 min, [M+H]⁺: 186

### Synthesis of Intermediates B1-B9

### Synthesis of Intermediates B1

### Step 1: Synthesis of 1-bromo-2-(bromomethyl)-3-nitrobenzene

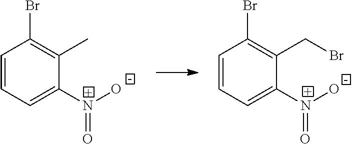

2-Bromo-6-nitrotoluene (50 g, 231 mmol) is dissolved in DCE (300 mL). A suspension of NBS (61.8 g, 347 mmol) in DCE (400 mL) is added at RT and the reaction mixture is stirred at reflux. Then AIBN (2.66 g, 16.2 mmol) in DCM (35 mL) is slowly added (syringe pump, pump rate approximately 1 drop per 6 seconds), and the reaction mixture is stirred at reflux overnight. The reaction mixture is concentrated, the residue is dissolved in DCM (500 mL) and washed 3× with water. The organic layer is dried over MgSO4 and filtered through a short plug of silica, and the silica is washed with DCM (50 ml). The filtrate is concentrated and dried in high vacuum to afford the desired product.

TLC: silica gel, CycH/EtOAc 5/1: R_{f}: 0.4

### Step 2: Synthesis of N-[(2-bromo-6-nitrophenyl)methyl]-1-cyclopropyl-3-methoxy-1H-pyrazol-4-amine

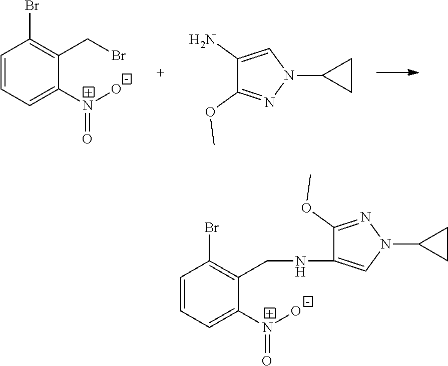

1-Cyclopropyl-3-methoxy-1H-pyrazol-4-amine hydrochloride (A1) (418 mg, 1.41 mmol) is dissolved in ACN (50 mL) and DIPEA (0.61 mL, 3.52 mmol) is added. 1-Bromo-2-(bromomethyl)-3-nitrobenzene (3.19 g, 10.8 mmol) is added in portions and the reaction mixture is stirred at RT overnight. The reaction is filtered and purified by reversed phase chromatography (HPLC; ACN/water/NH₃) to afford the desired compound.

Analysis (method A): Rₜ: 0.50 min, [M+H]⁺: 367

### Step 3: Synthesis of 4-bromo-2-(1-cyclopropyl-3-methoxy-1H-pyrazol-4-yl)-2H-indazole (Intermediate B1)

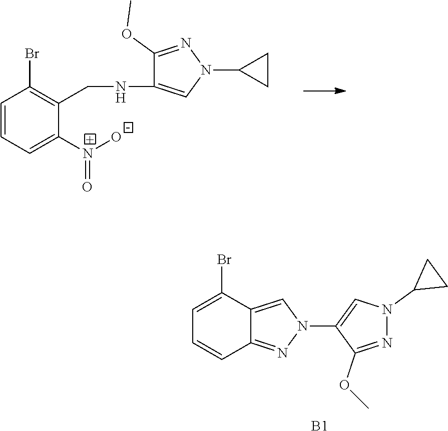

N-[(2-bromo-6-nitrophenyl)methyl]-1-cyclopropyl-3-methoxy-1H-pyrazol-4-amine (2.61 g, 7.12 mmol) is suspended in MeOH (40 mL). Zinc (2.33 g, 35.6 mmol) is added, a solution of ammonium formate (449 mg, 7.12 mmol) in MeOH (20 mL) is added dropwise, and the reaction mixture is stirred at RT overnight. The reaction mixture is diluted with DCM, filtered through a pad of Celite, washed with DCM, and concentrated. The residue is purified by reversed phase chromatography (HPLC; ACN/water/NH₃) to afford the intermediate B1.

Analysis (method A): Rₜ: 0.68 min, [M+H]⁺: 333

The intermediates compiled in the following table are obtained by following a reaction sequence analogous to that described for intermediate B1 using 1-bromo-2-(bromomethyl)-3-nitrobenzene and the corresponding hetaryl amines as starting material.

| | | | | MS | |
|---|---|---|---|---|---|
| | | | | (ESI+): | |
| Inter- | Hetaryl amine | | LC | m/z | t_{R} |
| mediate | and conditions | Structure/Name | Method | [M + H]⁺ | [min] |
| B2 | A2 DIPEA NMP 80° C. 2 h RT 16 h | 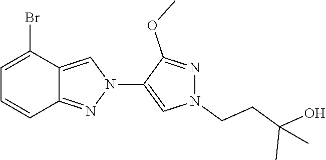 [SMILES: COc1nn(CCC(C)(C)O)cc1-n1cc2c(Br)cccc2n1] 4-[4-(4-bromo-2H-indazol-2-yl)-3- methoxy-1H-pyrazol-1-yl]-2- methylbutan-2-ol | B | 379 | 1.05 |
| B3 | 3-methoxy-1- methyl-1H- pyrazol-4-amine hydrochloride DIPEA ACN RT 16 h RT 16 h | 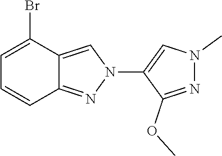 [SMILES: COc1nn(C)cc1-n1cc2c(Br)cccc2n1] 4-bromo-2-(3-methoxy-1-methyl- 1H-pyrazol-4-yl)-2H-indazole | A | 307 | 0.61 |
| B4 | 3-ethoxy-1- methyl-1H- pyrazol-4-amine DIPEA ACN RT 21 h RT 18 h | 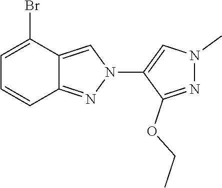 [SMILES: CCOc1nn(C)cc1-n1cc2c(Br)cccc2n1] 4-bromo-2-(3-ethoxy-1-methyl-1H- pyrazol-4-yl)-2H-indazole | C | 321 | 0.99 |
| B7 | A4 DIPEA NMP 80 C° 1 h RT 16 h | 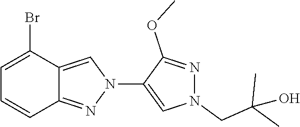 [SMILES: COc1nn(CC(C)(C)O)cc1-n1cc2c(Br)cccc2n1] 1-[4-(4-bromo-2H-indazol-2-yl)-3- methoxy-1H-pyrazol-1-yl]-2- methylpropan-2-ol | B | 365 | 1.04 |

### Synthesis of Intermediate B5

### Step 1: Synthesis of 1-bromo-2-(bromomethyl)-3-nitrobenzene

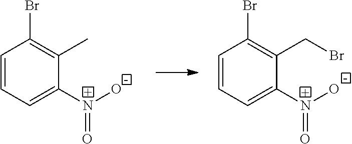

2-Bromo-6-nitrotoluene (50 g, 231 mmol) is dissolved in DCE (300 mL). A suspension of NBS (61.8 g, 347 mmol) in DCE (400 mL) is added at RT and the reaction mixture is stirred at reflux. Then AIBN (2.66 g, 16.2 mmol) in DCM (35 mL) is slowly added (syringe pump, pump rate approximately 1 drop per 6 seconds), and the reaction mixture is stirred at reflux overnight. The reaction mixture is concentrated, the residue is dissolved in DCM (500 mL) and washed 3× with water. The organic layer is dried over MgSO4 and filtered through a short plug of silica, and the silica is washed with DCM (50 ml). The filtrate is concentrated and dried in high vacuum to afford the desired product.

TLC: silica gel, CycH/EtOAc 5/1: R_{f}: 0.4

### Step 2: Synthesis of N-[(2-bromo-6-nitrophenyl)methyl]-5-(difluoromethyl)-1-methyl-1H-pyrazol-3-amine

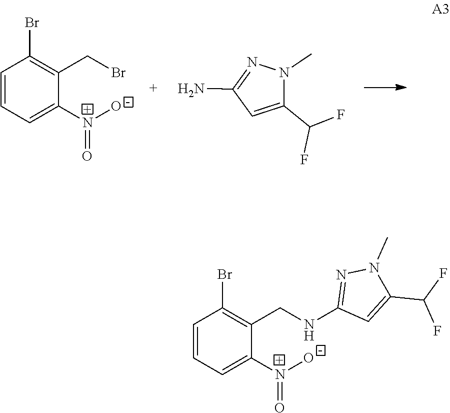

Under an atmosphere of argon, 5-(difluoromethyl)-1-methyl-1H-pyrazol-3-amine hydrochloride (A3) (0.36 g, 1.96 mmol) and DIPEA (1.70 mL, 9.83 mmol) are dissolved in NMP (7 mL). 1-Bromo-2-(bromomethyl)-3-nitrobenzene (0.75 g, 2.55 mmol) is added, and the reaction mixture is stirred at 80° C. for 3 h. The reaction mixture is diluted with MeTHF (90 mL) and water (70 mL) and is extracted. The organic layer is washed with water, dried, filtered, and concentrated. The residue is purified by flash chromatography (CycH/EtOAc 100/0→CycH/EtOAc 30/70) to afford the desired compound.

Analysis (method C): Rₜ: 0.85 min, [M+H]⁺: 361

### Step 3: Synthesis of 4-bromo-2-[5-(difluoromethyl)-1-methyl-1H-pyrazol-3-yl]-2H-indazole (Intermediate B5)

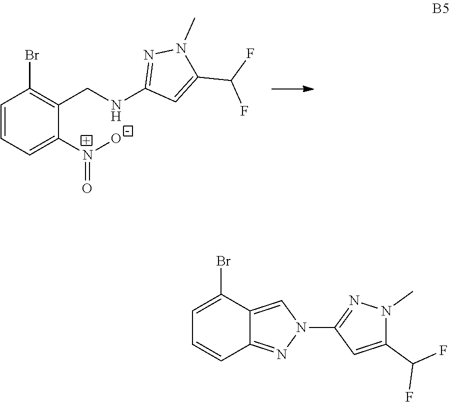

N-[(2-bromo-6-nitrophenyl)methyl]-5-(difluoromethyl)-1-methyl-1H-pyrazol-3-amine (440 mg, 0.91 mmol, 75% purity) is dissolved in MeOH (10 mL). Zinc (400 mg, 6.12 mmol) is added, a solution of ammonium formate (70 mg, 1.11 mmol) in MeOH (5 mL) is added dropwise over a period of 10 min, and the reaction mixture is stirred at RT for 20 h. More ammonium formate (70 mg, 1.11 mmol) is added, and the reaction mixture is stirred at RT for 3 h. The reaction mixture is diluted with DCM (15 mL) and an aqueous saturated solution of NaHCO₃ (0.2 mL) and stirred a few minutes. Then it is filtered, and the filtrate is concentrated. The residue is crystallized with ACN/water (2 mL each) and the precipitate is filtered to afford the intermediate B5.

Analysis (method C): Rₜ: 0.98 min, [M+H]⁺: 327

### Synthesis of Intermediate B6

### Step 1: Synthesis of 1-bromo-2-(bromomethyl)-3-nitrobenzene

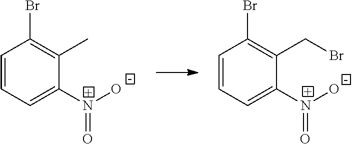

2-Bromo-6-nitrotoluene (50 g, 231 mmol) is dissolved in DCE (300 mL). A suspension of NBS (61.8 g, 347 mmol) in DCE (400 mL) is added at RT and the reaction mixture is stirred at reflux. Then AIBN (2.66 g, 16.2 mmol) in DCM (35 mL) is slowly added (syringe pump, pump rate approximately 1 drop per 6 seconds), and the reaction mixture is stirred at reflux overnight. The reaction mixture is concentrated, the residue is dissolved in DCM (500 mL) and washed 3× with water. The organic layer is dried over MgSO4 and filtered through a short plug of silica, and the silica is washed with DCM (50 ml). The filtrate is concentrated and dried in high vacuum to afford the desired product.

TLC: silica gel, CycH/EtOAc 5/1: R_{f}: 0.4

### Step 2: Synthesis of N-[(2-bromo-6-nitrophenyl)methyl]-1-methyl-5-(trifluoromethyl)-1H-pyrazol-3-amine

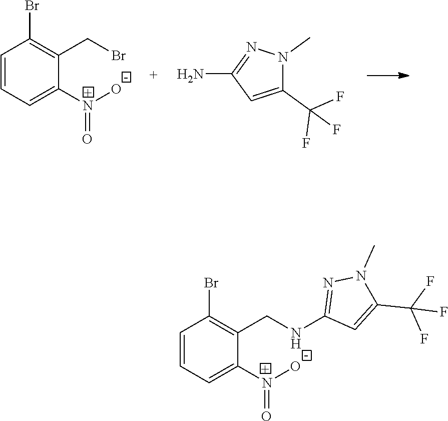

1-Methyl-5-(trifluoromethyl)-1H-pyrazol-3-amine hydrochloride (2.03 g, 10.1 mmol) and K₂CO₃ (1.39 g, 10.1 mmol) are dissolved in NMP (40 mL). DIPEA (3.48 mL, 20.1 mmol) and 1-bromo-2-(bromomethyl)-3-nitrobenzene (2.00 g, 6.71 mmol) are added, and the reaction mixture is stirred at 80° C. for 6 h. The reaction mixture is quenched by the addition of water (600 mL) and saturated NH₄Cl. The formed precipitate is filtered washed with water, dissolved in EtOAc, dried over MgSO₄, filtered, and concentrated to afford the desired compound.

Analysis (method E): Rₜ: 1.07 min, [M+H]⁺: 379

### Step 3: Synthesis of 4-bromo-2-[1-methyl-5-(trifluoromethyl)-1H-pyrazol-3-yl]-2H-indazole (Intermediate B6)

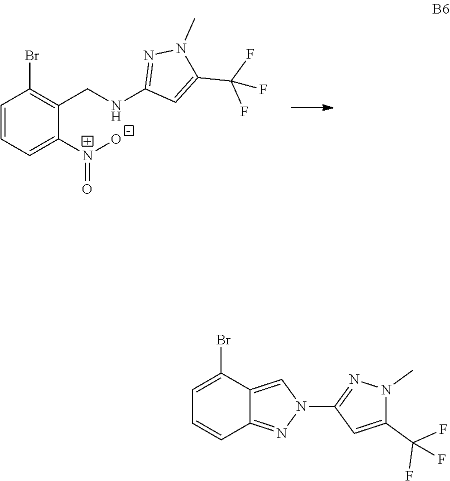

N-[(2-bromo-6-nitrophenyl)methyl]-1-methyl-5-(trifluoromethyl)-1H-pyrazol-3-amine (290 mg, 0.76 mmol) is dissolved in MeOH (15 mL). Zinc (152 mg, 2.27 mmol) is added, a solution of ammonium formate (66.9 mg, 1.06 mmol) in MeOH (5 mL) is added dropwise, and the reaction mixture is stirred at RT overnight. More zinc (75 mg, 1.12 mmol) ammonium formate (30 mg, 0.48 mmol) are added, and the reaction mixture is stirred at RT for 3 h. The reaction mixture is diluted with DCM (20 mL), filtered through a pad of Celite, washed with DCM/MeOH 1/1, and concentrated. The residue is purified by reversed phase chromatography (HPLC; ACN/water/NH₃) to afford the intermediate B6.

Analysis (method E): Rₜ: 1.15 min, [M+H]⁺: 345

### Synthesis of Intermediate B8

### Step 1: Synthesis of N-(2-amino-3-bromophenyl)-1-methyl-5-(trifluoromethyl)-1H-pyrazole-3-carboxamide

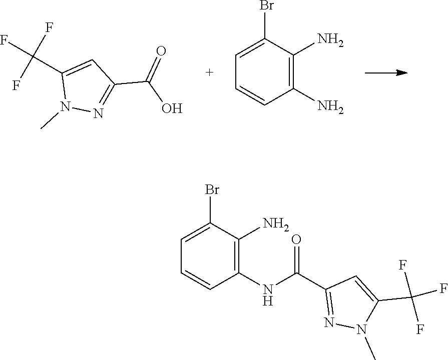 [SMILES: Cn1nc(C(=O)O)cc1C(F)(F)F ; Nc1cccc(Br)c1N ; Cn1nc(C(=O)Nc2cccc(Br)c2N)cc1C(F)(F)F]

1-methyl-5-(trifluoromethyl)-1H-pyrazole-3-carboxylic acid (800 mg, 3.92 mmol), 3-bromobenzene-1,2-diamine (830 mg, 4.31 mmol), DIPEA (1.69 mL, 9.79 mmol) and HATU (1.94 g, 5.09 mmol) is dissolved in DMF (15 mL). The reaction mixture is stirred at RT overnight. The reaction mixture is quenched with 150 mL water, stirred for 20 min and the precipitation is filtered, washed with water and dried to afford the desired product.

Analysis (method N): Rₜ: 0.9 min, [M+H]⁺: 363

### Step 2: Synthesis of 4-bromo-2-[1-methyl-5-(trifluoromethyl)-1H-pyrazol-3-yl]-1H-1,3-benzodiazole (Intermediate B8)

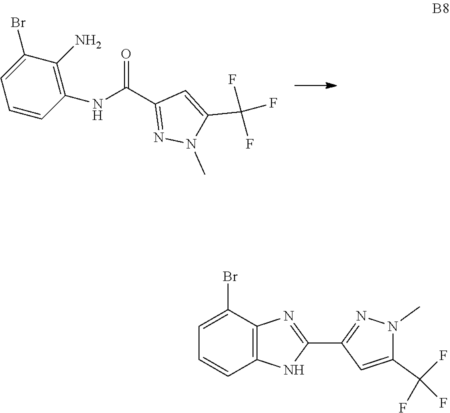 [SMILES: Cn1nc(C(=O)Nc2cccc(Br)c2N)cc1C(F)(F)F ; Cn1nc(-c2nc3c(Br)cccc3[nH]2)cc1C(F)(F)F]

N-(2-Amino-3-bromophenyl)-1-methyl-5-(trifluoromethyl)-1H-pyrazole-3-carboxamide (1.42 g, 3.72 mmol) is dissolved in Acetic acid (15 mL). The reaction mixture is stirred at 105° C. for 2 h. The reaction mixture is diluted with water, stirred for 5 min and the precipitation is filtered, washed with water, and dried to afford the intermediate B8.

Analysis (method N): Rₜ: 0.80 min, [M+H]⁺: 345

### Synthesis of Intermediate B9

### Step 1: Synthesis of (E)-1-(2-bromo-6-nitrophenyl)-N-(5-cyclopropyl-1-methyl-1H-pyrazol-3-yl)methanimine

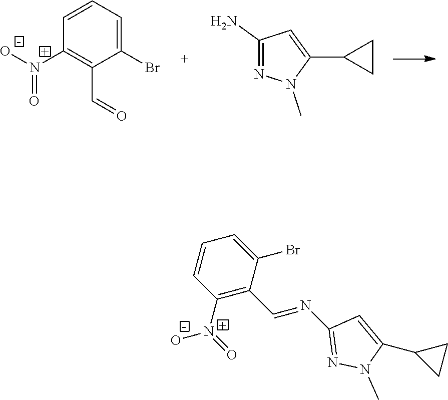

2-Bromo-6-nitrobenzaldehyde (1.60 g, 6.74 mmol), 5-cyclopropyl-1-methyl-1H-pyrazol-3-amine (1.03 g, 7.13 mmol) and mole sieve 3A (2.00 g) is dissolved in methanol (20 mL). The reaction mixture is stirred at RT for 16 h. Acetic acid (0.773 ml, 13.4 mmol) is added to the reaction mixture and is stirred at RT for 2 h. The reaction mixture is filtered and the filtrate is concentrated to afford the desired product.

Analysis (method D): Rₜ: 0.66 min, [M+H]⁺: 349

### Step 2: Synthesis of 4-bromo-2-(5-cyclopropyl-1-methyl-1H-pyrazol-3-yl)-2H-indazole (Intermediate B9)

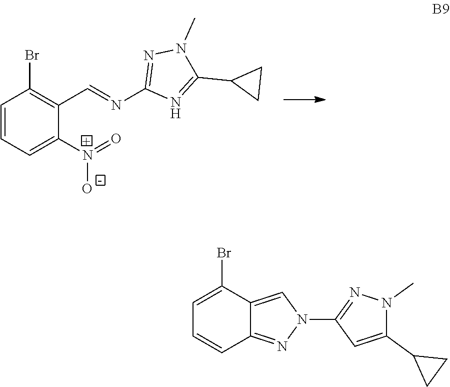

(E)-1-(2-Bromo-6-nitrophenyl)-N-(5-cyclopropyl-1-methyl-1H-pyrazol-3-yl)methanimine (2.70 g, 6.72 mmol) is dissolved in triethyl phosphite (15 mL). The reaction mixture is stirred at 150° C. for 45 min. The reaction mixture is concentrated, and the residue is purified by reversed phase chromatography (HPLC; ACN/water/NH₃) to afford the intermediate B9.

Analysis (method A): Rₜ: 0.72 min, [M+H]⁺: 317

### Synthesis of Intermediate B10 (Mixture of Regioisomers)

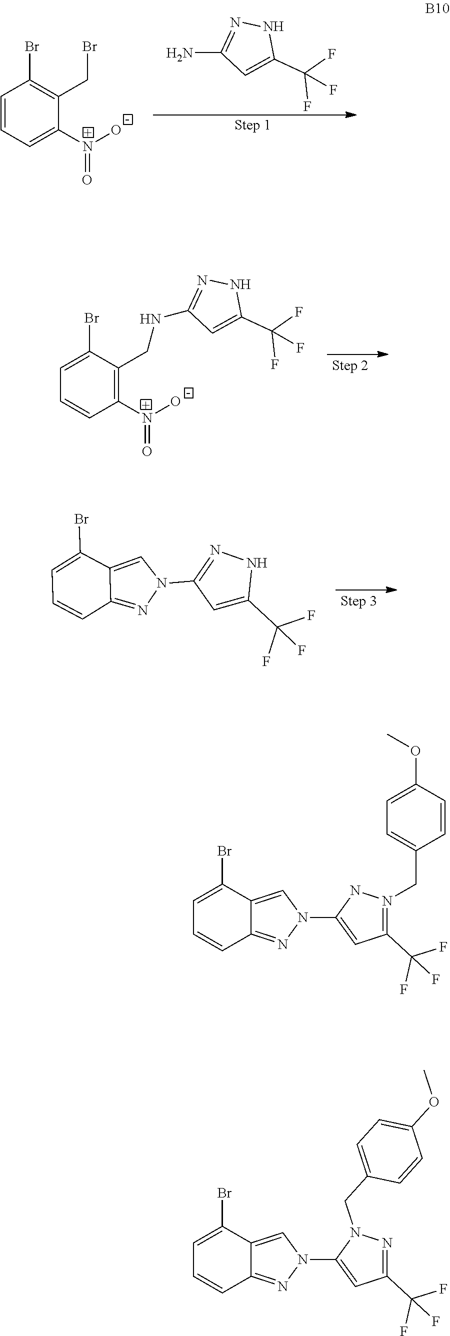

### Step 1

1-Bromo-2-(bromomethyl)-3-nitrobenzene (2.5 g; 8.05 mmol) is dissolved in acetonitrile (50 mL) and 5-(trifluoromethyl)-1H-pyrazol-3-amine (2.56 g, 16 mmol) is added and the mixture stirred for 10 h at 80° C. The mixture is concentrated and extracted with water and ethylacetate and the organic phase concentrated and purified via reversed phase chromatography.

Analysis (method N): Rₜ: 0.81 min, [M+H]⁺: 365

### Step 2

N-[(2-Bromo-6-nitrophenyl)methyl]-5-(trifluoromethyl)-1H-pyrazol-3-amine (1.7 g; 3 mmol), Zn nanopowder (1.38 g, 21 mmol) and MeOH (5 mL) are combined and ammonium formate (0.23 g dissolved in MeOH (5 mL) is added dropwise over 60 min and stirred for 17 h. The mixture is filtered and the filtrate concentrated.

Analysis (method N): Rₜ: 0.97 min, [M−H]⁻: 329

### Step 3

4-Bromo-2-[5-(trifluoromethyl)-1H-pyrazol-3-yl]-2H-indazole (430 mg, 1.23 mmol) is dissolved in DMF (5 mL) and NaH (113 mg, 2.6 mmol) is added at RT and stirred for 15 min. Then 4-methoxybenzyl chloride (0.234 mL, 1.67 mmol) is added and the mixture stirred for 6 h at RT. The mixture is diluted with water and extracted with ethylacetate. The organic phase is concentrated and the product purified via silica gel chromatography (CycH/EE gradient 95/5→8/2) to afford intermediate B10

Analysis (method N): Rₜ: 1.18 min and 1.29, [M−H]⁻: 451 (mixture of regioisomers)

### Synthesis of Intermediates C1-C12

### Synthesis of Intermediate C1

### Synthesis of [2-(1-cyclopropyl-3-methoxy-1H-pyrazol-4-yl)-2H-indazol-4-yl]boronic acid (Intermediate C1)

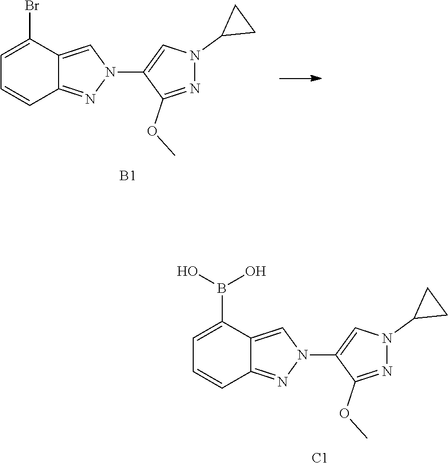 [SMILES: COc1nn(C2CC2)cc1-n1cc2c(Br)cccc2n1 ; "B1" ; COc1nn(C2CC2)cc1-n1cc2c(B(O)O)cccc2n1 ; "C1"]

4-Bromo-2-(1-cyclopropyl-3-methoxy-1H-pyrazol-4-yl)-2H-indazole (B1) (700 mg, 2.10 mmol) is dissolved in dioxane (10 mL). Bis(neopentyl glycolato)diboron (1.47 g, 6.30 mmol) and potassium acetate (1.03 g, 10.5 mmol) are added, and the mixture is purged with argon. Pd(dppf)Cl₂×DCM (85.8 mg, 0.11 mmol) is added, and the reaction mixture is stirred at 80° C. for 3 h. After the reaction mixture is cooled to RT the reaction mixture is diluted with ACN, filtered, and purified by reversed phase chromatography (HPLC; ACN/water/TFA) to afford the intermediate C1.

Analysis (method A): Rₜ: 0.41 min, [M+H]⁺: 299

### Synthesis of Intermediate C2

### Synthesis of 4-{3-methoxy-4-[4-(4,4,5,5-tetramethyl-1,3,2-dioxaborolan-2-yl)-2H-indazol-2-yl]-1H-pyrazol-1-yl}-2-methylbutan-2-ol (Intermediate C2)

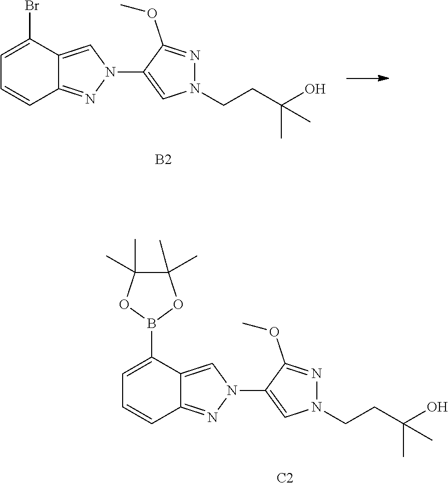

4-[4-(4-Bromo-2H-indazol-2-yl)-3-methoxy-1H-pyrazol-1-yl]-2-methylbutan-2-ol (B2) (300 mg, 0.79 mmol) is dissolved in dioxane (5 mL). Bis(pinacolato)diboron (261 mg, 1.03 mmol) and potassium acetate (233 mg, 2.37 mmol) are added, and the mixture is purged with argon. Pd(dppf)Cl₂×DCM (64.6 mg, 0.08 mmol) is added, and the reaction mixture is stirred at 100° C. for 2 h. After the reaction mixture is cooled to RT the precipitate is filtered, washed with dioxane (5 mL), and the filtrate is concentrated afford the intermediate C2.

Analysis (method E): Rₜ: 1.15 min, [M+H]⁺: 427

### Synthesis of Intermediate C3

### Synthesis of 4-(5,5-dimethyl-1,3,2-dioxaborinan-2-yl)-2-(3-methoxy-1-methyl-1H-pyrazol-4-yl)-2H-indazole (Intermediate C3)

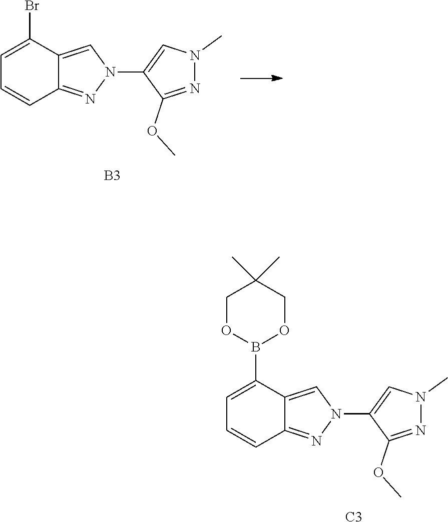 [SMILES: COc1nn(C)cc1-n1cc2c(Br)cccc2n1 ; "B3" ; COc1nn(C)cc1-n1cc2c(B3OCC(C)(C)CO3)cccc2n1 ; "C3"]

4-Bromo-2-(3-methoxy-1-methyl-1H-pyrazol-4-yl)-2H-indazole (B3) (0.63 g, 1.83 mmol, 90% purity) is dissolved in dioxane (10 mL). Bis(neopentyl glycolato)diboron (0.62 g, 2.75 mmol) and potassium acetate (0.54 g, 5.49 mmol) are added, and the mixture is purged with argon. Pd(dppf)Cl₂×DCM (0.22 g, 0.28 mmol) is added, and the reaction mixture is stirred at 90° C. for 1.5 h. After the reaction mixture is cooled to RT, it is diluted with iPrOAc (15 mL) and is purified by flash chromatography (DCM/MeOH 100/0→DCM/MeOH 90/10) to afford the intermediate C3.

Analysis (method C) Rₜ: 0.51 min, [M+H]⁺: 273 (boronic acid)

The intermediates compiled in the following table are obtained by following a reaction sequence analogous to that described for intermediate C2 or C3.

| | | | | MS | |
|---|---|---|---|---|---|
| | | | | (ESI+): | |
| Inter- | Starting materials | | LC | m/z | t_{R} |
| mediate | and conditions | Structure/Name | Method | [M + H]⁺ | [min] |
| C4 | B4 + Bis(neopentyl glycolato)diboron KOAc Pd(dppf)Cl₂ × DCM 90° C. 2.5 h | 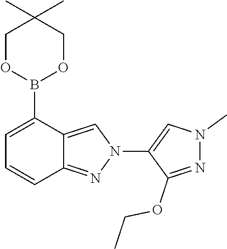 [SMILES: CCOc1nn(C)cc1-n1cc2c(B3OCC(C)(C)CO3)cccc2n1] 4-(5,5-dimethyl-1,3,2- dioxaborinan-2-yl)-2-(3-ethoxy- 1-methyl-1H-pyrazol-4-yl)-2H- indazole | D | 287 (boronic acid) | 0.25 |
| C6 | B6 + Bis(pinacolato) diboron KOAc Pd(dppf)Cl₂ × DCM 100° C. 2 h | 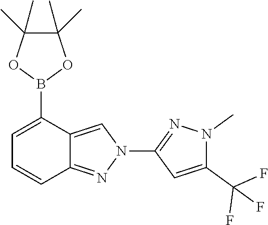 [SMILES: Cn1nc(-n2cc3c(B4OC(C)(C)C(C)(C)O4)cccc3n2)cc1C(F)(F)F] 2-[1-methyl-5-(trifluoromethyl)- 1H-pyrazol-3-yl]-4-(4,4,5,5- tetramethyl-1,3,2-dioxaborolan- 2-yl)-2H-indazole | E | 393 | 1.29 |
| C8 | B7 + Bis(pinacolato) diboron KOAc Pd(dppf)Cl₂ × DCM 100° C. 2 h | 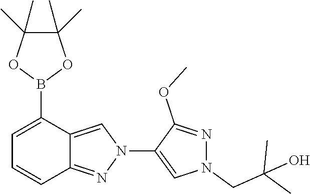 [SMILES: COc1nn(CC(C)(C)O)cc1-n1cc2c(B3OC(C)(C)C(C)(C)O3)cccc2n1] 1-{3-methoxy-4-[4-(4,4,5,5- tetramethyl-1,3,2-dioxaborolan- 2-yl)-2H-indazol-2-yl]-1H- pyrazol-1-yl}-2-methylpropan-2- ol | E | 413 | 1.13 |

### Synthesis of Intermediate C5

### Synthesis of {2-[5-(difluoromethyl)-1-methyl-1H-pyrazol-3-yl]-2H-indazol-4-yl}boronic acid (Intermediate C5)

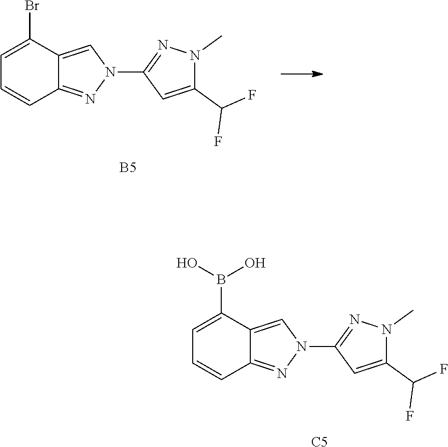

4-Bromo-2-[5-(difluoromethyl)-1-methyl-1H-pyrazol-3-yl]-2H-indazole (B5) (240 mg, 0.73 mmol) is dissolved in dioxane (6 mL). Bis(neopentyl glycolato)diboron (249 mg, 1.10 mmol) and potassium acetate (216 mg, 2.20 mmol) are added, and the mixture is purged with argon. Pd(dppf)Cl₂×DCM (159 mg, 0.19 mmol) is added, and the reaction mixture is stirred at 100° C. for 2 h. After the reaction mixture is cooled to RT the reaction mixture is concentrated, diluted with DMF, filtered, and purified by reversed phase chromatography (HPLC; ACN/water/TFA) to afford the intermediate C5.

Analysis (method E): Rₜ: 0.78 min, [M+H]⁺: 293

### Synthesis of Intermediate C7

### Synthesis of 4-(5,5-dimethyl-1,3,2-dioxaborinan-2-yl)-2-[1-methyl-5-(trifluoromethyl)-1H-pyrazol-3-yl]-2H-indazole (Intermediate C7)

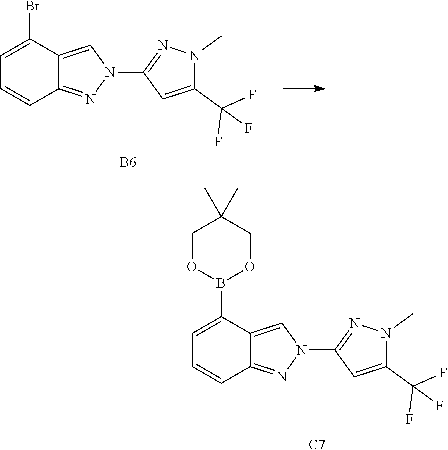

4-Bromo-2-[1-methyl-5-(trifluoromethyl)-1H-pyrazol-3-yl]-2H-indazole (B6) (1.00 g, 2.90 mmol) is dissolved in dioxane (15 mL). Bis(neopentyl glycolato)diboron (0.98 g, 4.35 mmol) and potassium acetate (0.85 g, 8.69 mmol) are added, and the mixture is purged with argon. Pd(dppf)Cl₂×DCM (0.71 g, 0.87 mmol) is added, and the reaction mixture is stirred at 100° C. for 3 h. After the reaction mixture is cooled to RT, it is filtered through a pad of Celite and is purified by flash chromatography (hexane/acetone 100/0→hexane/acetone 0/100). The residue is triturated with pentane/diethyl ether to afford the intermediate C7.

Analysis (method F): Rₜ: 2.65 min, [M+H]⁺: 311 (boronic acid)

### Synthesis of Intermediate C9

### Synthesis of 2-[5-(difluoromethyl)-1-methyl-1H-pyrazol-3-yl]-4-(4,4,5,5-tetramethyl-1,3,2-dioxaborolan-2-yl)-2H-indazole (Intermediate C9)

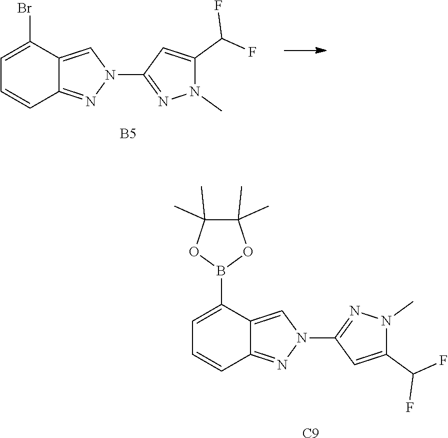

4-Bromo-2-[5-(difluoromethyl)-1-methyl-1H-pyrazol-3-yl]-2H-indazole (B5) (15.0 g, 44.6 mmol), Bis(pinacolato)diboron (12.5 g, 49.0 mmol), potassium acetate (14.0 g, 142 mmol), Pd(dppf)Cl₂×DCM (2.00 g, 2.45 mmol) are dissolved in dioxane (200 mL). The mixture is purged with argon and is stirred at 90° C. for 3 h. After the reaction mixture is cooled to RT, it is diluted with DCM (200 mL), filtered, and concentrated to afford the intermediate C9.

Analysis (method C): Rₜ: 1.16 min, [M+H]⁺: 375

### Synthesis of Intermediate C10

### Synthesis of 2-[1-methyl-5-(trifluoromethyl)-1H-pyrazol-3-yl]-4-(4,4,5,5-tetramethyl-1,3,2-dioxaborolan-2-yl)-1H-1,3-benzodiazole (Intermediate C10)

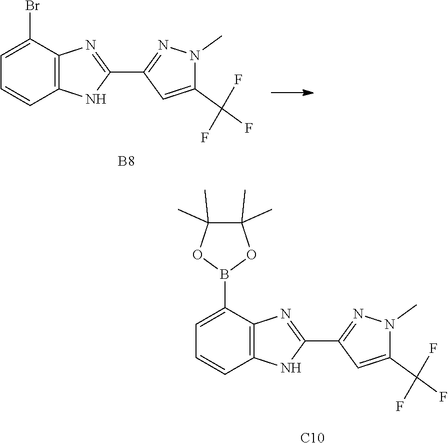

4-Bromo-2-[1-methyl-5-(trifluoromethyl)-1H-pyrazol-3-yl]-1H-1,3-benzodiazole (B8) (1.30 g, 3.77 mmol) is dissolved in dioxane (50 mL). Bis(pinacolato)diboron (1.45 g, 5.65 mmol) and potassium acetate (1.12 g, 11.3 mmol) are added, and the mixture is purged with argon. Pd(dppf)Cl₂×DCM (153 mg, 0.19 mmol) is added, and the reaction mixture is stirred at 80° C. for 3 h. After the reaction mixture is cooled to RT the reaction mixture is filtered over celite and concentrated. The residue is extracted with EtOAc and sat. NaCl solution. The organic layer is dried over MgSO₄, filtered, and concentrated. The residue is purified by reversed phase chromatography (HPLC; ACN/water/formic acid) to afford the intermediate C10.

Analysis (method N): Rₜ: 0.67 min, [M+H]⁺: 311

### Synthesis of Intermediate C11

### Synthesis of 2-(5-cyclopropyl-1-methyl-1H-pyrazol-3-yl)-4-(5,5-dimethyl-1,3,2-dioxaborinan-2-yl)-2H-indazole (Intermediate C11)

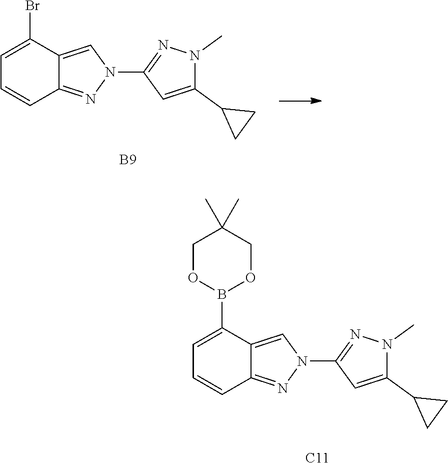

4-Bromo-2-(5-cyclopropyl-1-methyl-1H-pyrazol-3-yl)-2H-indazole 4 (B9) (950 mg, 2.99 mmol), bis(neopentylglycolato)diboron (1.39 g, 5.99 mmol), potassium acetate (0.88 g, 8.98 mmol) and Pd(dppf)Cl₂×DCM (244 mg, 0.300 mmol) are dissolved in dioxane (15 mL). The mixture is purged with argon and is stirred at 80° C. for 3 h. After the reaction mixture is cooled to RT the reaction mixture is absorbed on diatomaceous earth and is purified by flash chromatography (CH/EtOAc 100/0→0/100). The residue is diluted in n-heptane, sonicated for 5 min and the precipitation is filtered to afford the intermediate C11.

Analysis (method A): Rₜ: 0.45 min, [M+H]⁺: 351

### Synthesis of Intermediate C12

### Synthesis of {2-[1-methyl-5-(trifluoromethyl)-1H-pyrazol-3-yl]-2H-indazol-4-yl}boronic acid (Intermediate C12)

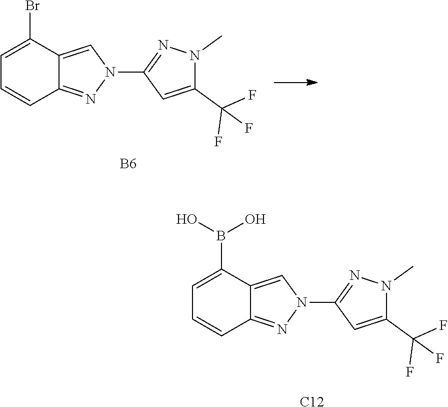

4-Bromo-2-[1-methyl-5-(trifluoromethyl)-1H-pyrazol-3-yl]-2H-indazole (B6) (475 mg, 1.37 mmol), Bis(neopentylglycolato)diboron (466 mg, 2.06 mmol), potassium acetate (405 mg, 2.06 mmol) and Pd(dppf)Cl₂×DCM (279 mg, 0.365 mmol) are dissolved in dioxane (15 mL). The mixture is purged with argon and is stirred at 100° C. for 2.5 h. After the reaction mixture is cooled to RT, filtered, and concentrated. The residue is purified by reversed phase chromatography (HPLC; ACN/water/TFA) to afford the intermediate C12.

Analysis (method E): Rₜ: 0.87 min, [M+H]⁺: 311

### Synthesis of Intermediate C13

### Synthesis of 2-{1-[(4-methoxyphenyl)methyl]-5-(trifluoromethyl)pyrazol-3-yl}-4-(4,4,5,5-tetramethyl-1,3,2-dioxaborolan-2-yl)indazole and 2-{2-[(4-methoxyphenyl)methyl]-5-(trifluoromethyl)pyrazol-3-yl}-4-(4,4,5,5-tetramethyl-1,3,2-dioxaborolan-2-yl)indazole (Intermediate C13)

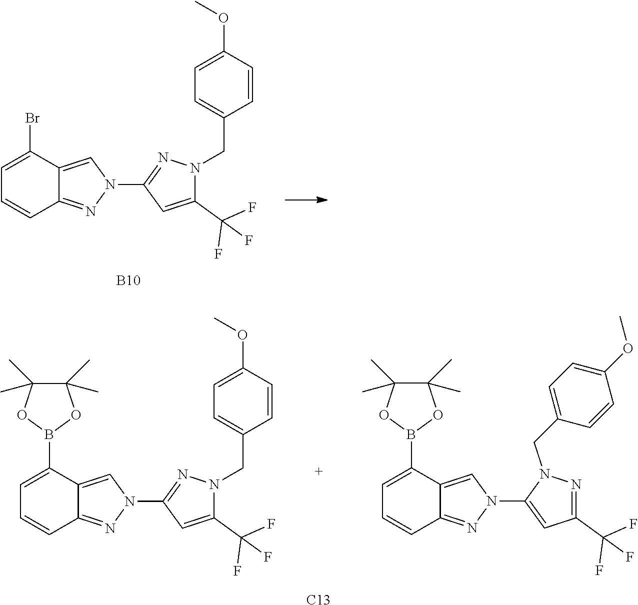

4-Bromo-2-{1-[(4-methoxyphenyl)methyl]-5-(trifluoromethyl)pyrazol-3-yl}indazole (B10) (550 mg, 1.29 mmol) is dissolved in acetonitril (10 mL). Bis(pinacolato)diboron (625 mg, 2.44 mmol) and potassium pivalate (540 mg, 3.7 mmol) are added, and the mixture is purged with argon. Pd(dppf)Cl₂×DCM (100 mg, 0.12 mmol) is added, and the reaction mixture is stirred at 80° C. for 3 h. After the reaction mixture is cooled to RT the reaction mixture is. extracted with EtOAc and water. The organic layer is dried over to afford the intermediate C13.

Analysis (method E): Rₜ: 1.00 and 1.25 min

### Synthesis of Intermediates D1 and D2

### Synthesis of Intermediate D1

### Step 1: Synthesis of 5-cyclopropyl-1-methyl-1H-imidazole

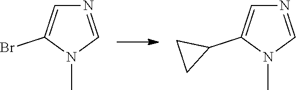

Under an argon atmosphere, 5-bromo-1-methyl-1H-imidazole (7.50 g, 46.6 mmol), cyclopropyl zinc bromide (132 mL, 65.9 mmol, 0.5 M in THF) and Pd(dppf)Cl₂ (2.20 g, 3.01 mmol) are mixed together, and the reaction mixture is stirred at 70° C. for 20 h. The reaction mixture is concentrated, and the residue is purified by flash chromatography (DCM/MeOH 100/0→DCM/MeOH 90/10) to afford the desired compound.

Analysis (method D): Rₜ: 0.31 min, [M+H]⁺: 123

### Step 2: Synthesis of 2,4-dibromo-5-cyclopropyl-1-methyl-1H-imidazole (Intermediate D1)

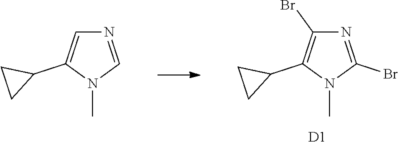

5-Cyclopropyl-1-methyl-1H-imidazole (9.20 g, 52.7 mmol) is dissolved in ACN (200 mL). At −5° C. NBS (18.8 g, 105 mmol) is added in portions and the reaction mixture is stirred at −5° C. for 30 min and at RT for 4 h. The reaction mixture is quenched by the addition of a saturated Na₂S₂O₃ solution (40.7 mL, 4.4 M). The formed precipitate is filtered and washed with ACN. The filtrate is extracted with EtOAc and the organic layer is dried (Na₂SO₄), filtered and concentrated. The residue is purified by flash chromatography (CycH/EtOAc 95/5→CycH/EtOAc 65/35) to afford the intermediate D1.

Analysis (method C): Rₜ: 0.77 min, [M+H]⁺: 279

### Synthesis of Intermediate D2

### Synthesis of 5-iodo-3-methyl-4-(propan-2-yl)-1H-pyrazole (Intermediate D2)

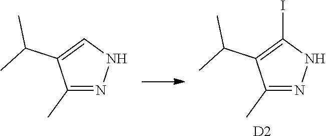

3-Methyl-4-(propan-2-yl)-1H-pyrazole (10 g, 80.5 mmol) is dissolved in ACN (150 mL). NIS (25 g, 111 mmol) is added, and the reaction mixture is stirred at 80° C. overnight. The reaction mixture is filtered, and the filtrate is evaporated. The residue is quenched with a half saturated Na₂S₂O₃ solution and extracted three times with DCM. The combined organic layers are dried (Na₂SO₄), filtered and concentrated. The crude product is purified by flash chromatography (CycH/EtOAc 95/5→CycH/EtOAc 76/24) to afford the intermediate D2.

Analysis (method E): Rₜ: 0.93 min, [M+H]⁺: 251

### Synthesis of Intermediates E1-E7

### Synthesis of Intermediate E1

### Synthesis of 4-chloro-N-methyl-3-(trifluoromethoxy)benzamide (Intermediate E1)

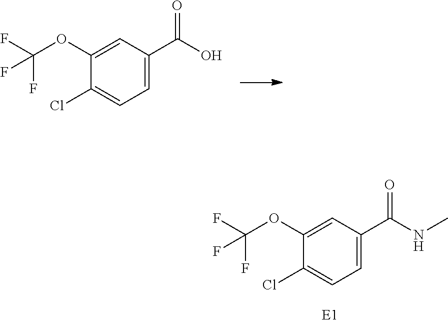 [SMILES: O=C(O)c1ccc(Cl)c(OC(F)(F)F)c1 ; CNC(=O)c1ccc(Cl)c(OC(F)(F)F)c1]

4-Chloro-3-(trifluoromethoxy)benzoic acid (1.00 g, 4.16 mmol) is dissolved in DMF (8 mL). DIPEA (2.20 mL, 12.7 mmol) and HATU (1.90 g, 5.00 mmol) are added, and the reaction mixture is stirred at rt for 10 min. Then methylamine (2 M in THF) (4.20 mL, 8.40 mmol) is added, and it is stirred at rt for 18 h. The reaction mixture is diluted with DCM (20 mL) and washed with an aqueous NaHCO₃ solution (1.1 M) (10 mL). The organic layer is dried and concentrated. The residue is purified by reversed phase chromatography (HPLC; ACN/water/NH₃) to afford the intermediate E1.

Analysis (method D): Rₜ: 0.56 min, [M+H]⁺: 254

### Synthesis of Intermediate E2 and E3

### Step 1: Synthesis of ethyl [4-chloro-3-(trifluoromethoxy)phenyl](methyl)phosphinate

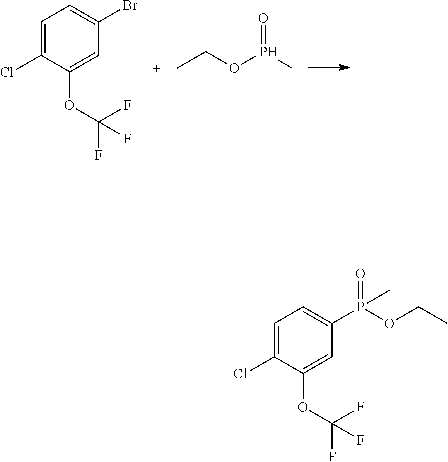

Under an argon atmosphere 4-bromo-1-chloro-2-(trifluoromethoxy)benzene (700 mg, 2.41 mmol) and ethyl methylphosphinate (604 mg, 5.31 mmol) are dissolved in ACN (5 mL) and DIPEA (1.67 mL, 9.66 mmol). Pd(PPh₃)₄ (279 mg, 0.24 mmol) is added and the reaction mixture is stirred at 85° C. overnight. The reaction mixture is filtered, and the filtrate is purified by reversed phase chromatography (HPLC; ACN/water/TFA) to obtain the title compound.

Analysis (method B): Rₜ: 0.93 min, [M+H]⁺: 303

### Step 2: Synthesis of 1-chloro-4-[ethyl(methyl)phosphoryl]-2-(trifluoromethoxy)benzene (Intermediate E2)

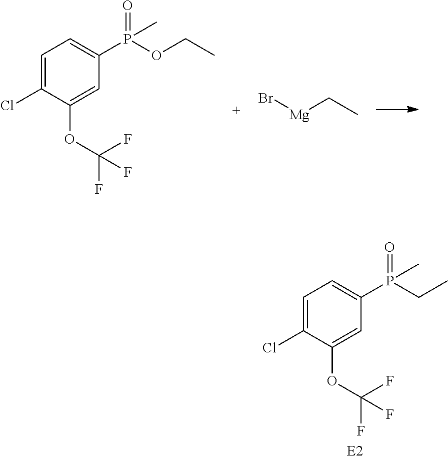

Under a nitrogen atmosphere ethyl [4-chloro-3-(trifluoromethoxy)phenyl](methyl)phosphinate (748 mg, 2.47 mmol) is dissolved in THF (10 mL). Ethyl magnesium bromide (2.91 mL, 3.4 M, 9.89 mmol) is added at −10° C. dropwise and the reaction mixture is stirred at RT overnight. Ethyl magnesium bromide (3.0 mL) is added at 0° C. dropwise and the reaction mixture is stirred at RT for 4 h. The reaction mixture is quenched with water and is extracted with EtOAc. The organic layer is dried (Na₂SO₄), filtered and concentrated to afford the intermediate E2.

Analysis (method E): Rₜ: 0.87 min, [M+H]⁺: 287

### Step 3: Synthesis of 1-chloro-4-[(S)-ethyl(methyl)phosphoryl]-2-(trifluoromethoxy)benzene (Intermediate E3)

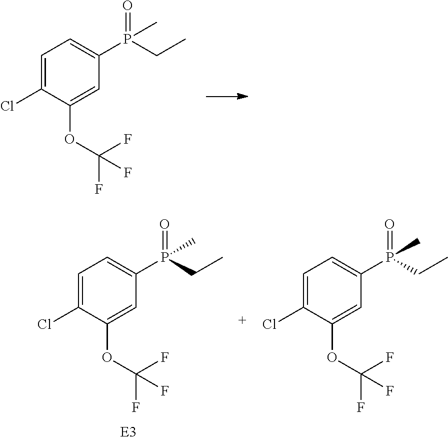 [SMILES: CCP(C)(=O)c1ccc(Cl)c(OC(F)(F)F)c1 ; CC[P@](C)(=O)c1ccc(Cl)c(OC(F)(F)F)c1 ; CC[P@@](C)(=O)c1ccc(Cl)c(OC(F)(F)F)c1]

1-Chloro-4-[ethyl(methyl)phosphoryl]-2-(trifluoromethoxy)benzene (2.94 g, 9.96 mmol) is separated by chiral purification method O to afford the compounds 1-chloro-4-[(S)-ethyl(methyl)phosphoryl]-2-(trifluoromethoxy)benzene E3 (analysis (method G): Rₜ: 2.49 min) and 1-chloro-4-[(R)-ethyl(methyl)phosphoryl]-2-(trifluoromethoxy)benzene (Analysis (method G): Rₜ: 2.71 min).

### Synthesis of Intermediate E4

### Synthesis of 1-chloro-4-(dimethylphosphoryl)-2-methylbenzene (Intermediate E4)

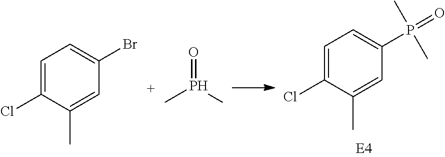

Under an argon atmosphere, 4-bromo-1-chloro-2-methylbenzene (2.00 g, 9.74 mmol) and (methylphosphonoyl)methane (840 mg, 10.8 mmol) are dissolved in ACN (40 mL) and DIPEA (6.74 mL, 39.0 mmol). Pd(PPh₃)₄ (1.12 g, 0.97 mmol) is added and the reaction mixture is stirred at 80° C. overnight. The reaction mixture is filtered, and the filtrate is purified by reversed phase chromatography (HPLC; ACN/water/TFA) to obtain the intermediate E4.

Analysis (method C): Rₜ: 0.57 min. [M+H]⁺: 203

### Synthesis of Intermediate E5

### Step 1: Synthesis of methyl 4-bromo-3-(hydroxymethyl)benzoate

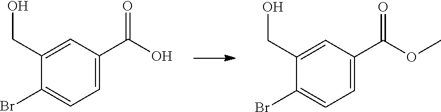

4-Bromo-3-(hydroxymethyl)benzoic acid (200 mg, 0.85 mmol) is dissolved in MeOH (5 mL). HCl (4 M in dioxane) (5 mL, 20.0 mmol) is added, and the reaction mixture is stirred at 70° C. for 3 h. The reaction mixture is concentrated to dryness to afford the desired compound.

Analysis (method E): Rₜ: 0.82 min, [M+H]⁺: 245

### Step 2: Synthesis of 4-bromo-3-(methoxymethyl)benzoic acid

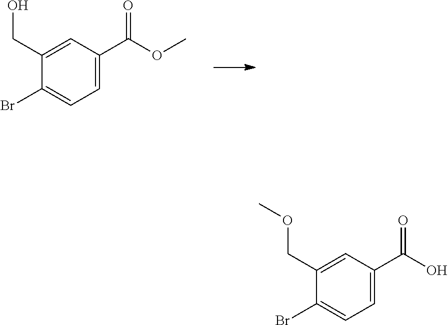

Methyl 4-bromo-3-(hydroxymethyl)benzoate (210 mg, 0.86 mmol) is dissolved in THF (5 mL). NaH (55%, 86.0 mg, 1.97 mmol) is added, and the reaction mixture is stirred at rt for 0.5 h. Then MeI (64.0 μL, 1.03 mmol) is added, and the reaction mixture is stirred at rt for 1 h. More NaH (55%, 86.0 mg, 1.97 mmol) and MeI (64.0 μL, 1.03 mmol) are added, and the reaction mixture is stirred at rt overnight. The reaction is diluted with EtOAc and washed with 1 M HCl and water. The organic layer is dried (MgSO₄), filtered, and evaporated to afford the titled compound.

Analysis (method H): Rₜ: 0.92 min, [M+H]⁺: 243

### Step 3: Synthesis of methyl 4-bromo-3-(methoxymethyl)benzoate (Intermediate E5)

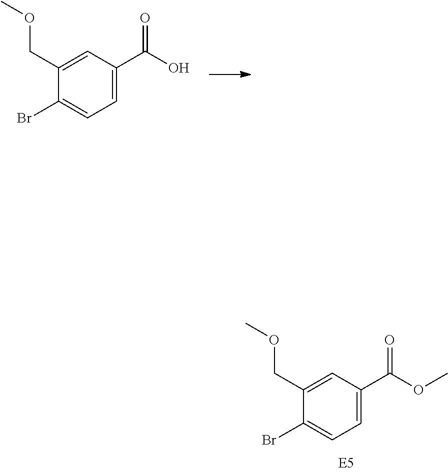

4-Bromo-3-(methoxymethyl)benzoic acid (210 mg, 0.86 mmol) is dissolved in MeOH (5 mL). HCl (4 M in dioxane) (5 mL, 20.0 mmol) is added, and the reaction mixture is stirred at 70° C. for 2 h. The reaction mixture is concentrated to dryness to afford the intermediate E5.

Analysis (method E): Rₜ: 0.99 min, [M+H]⁺: 259

### Synthesis of Intermediate E6

### Synthesis of 1-chloro-4-(diethylphosphoryl)-2-(trifluoromethoxy)benzene (Intermediate E6)

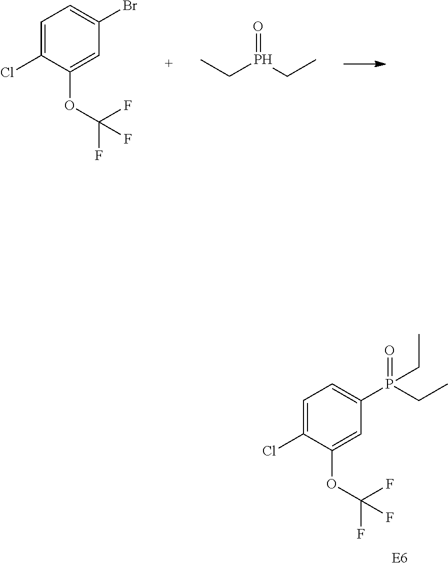

Under an argon atmosphere, 4-bromo-1-chloro-2-(trifluoromethoxy)benzene (1.00 g, 3.45 mmol) and (ethylphosphonoyl)ethane (700 mg, 6.27 mmol) are dissolved in ACN (5 mL) and DIPEA (2.50 mL, 14.5 mmol). Pd(PPh₃)₄ (1.40 g, 1.21 mmol) is added and the reaction mixture is stirred at 80° C. for 19.5 h. The reaction mixture is filtered, concentrated, and the residue is purified by reversed phase chromatography (HPLC; ACN/water/NH₃) to obtain the intermediate E6.

Analysis (method A): Rₜ: 0.55 min, [M+H]⁺: 301

### Synthesis of Intermediate E7

### Synthesis of 1-chloro-4-(dimethylphosphoryl)-2-(trifluoromethoxy)benzene (Intermediate E7)

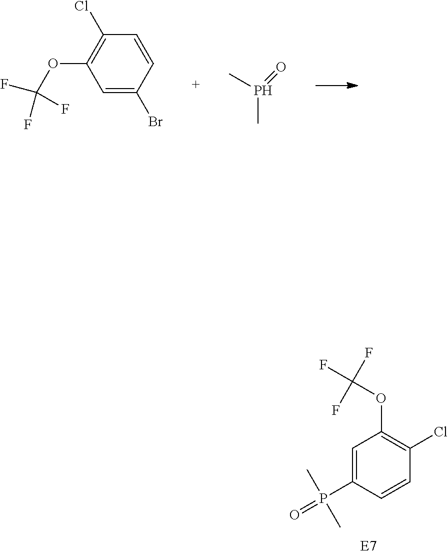

Under an argon atmosphere, 4-bromo-1-chloro-2-(trifluoromethoxy)benzene (1.00 g, 3.44 mmol) and (methylphosphonoyl)methane (425 mg, 5.17 mmol) are dissolved in ACN (5 mL) and DIPEA (2.40 mL, 13.8 mmol). Pd(PPh₃)₄ (400 mg, 0.34 mmol) is added and the reaction mixture is stirred at 80° C. for 24 h. The reaction mixture is filtered, and the filtrate is purified by reversed phase chromatography (HPLC; ACN/water/TFA) to obtain the intermediate E7.

Analysis (method D): Rₜ: 0.56 min, [M+H]⁺: 273

### Synthesis of Intermediates F1-F9

### Synthesis of Intermediate F1

### Synthesis of 4-(5,5-dimethyl-1,3,2-dioxaborinan-2-yl)-N-methyl-3-(trifluoromethoxy) benzamide (Intermediate F1)

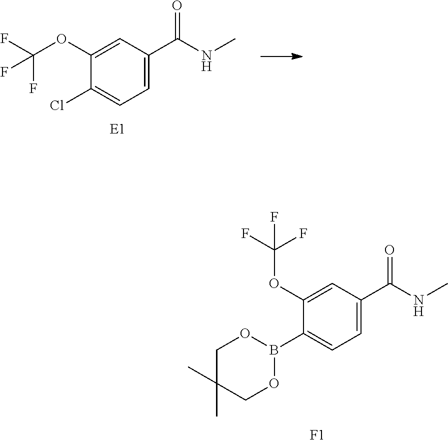

4-Chloro-N-methyl-3-(trifluoromethoxy)benzamide (E1) (750 mg, 2.96 mmol) is dissolved in dioxane (15 mL). Bis(neopentyl glycolato)diboron (1.20 g, 5.31 mmol) and potassium acetate (900 mg, 9.17 mmol) are added, and the mixture is purged with argon. XPhos Pd G3 (300 mg, 0.35 mmol) is added, and the reaction mixture is stirred at 100° C. for 1.5 h. After the reaction mixture is cooled to RT, it is diluted with ACN (20 mL), and the precipitate is filtered. The filtrate is concentrated, and the residue is triturated with MTBE. The formed precipitate is filtered and dried to afford the intermediate F1.

Analysis (method C): Rₜ: 0.42 min, [M+H]⁺: 264 (boronic acid)

### Synthesis of Intermediate F2

### Synthesis of 4-(4,4,5,5-tetramethyl-1,3,2-dioxaborolan-2-yl)-3-(trifluoromethoxy)benzonitrile (Intermediate F2)

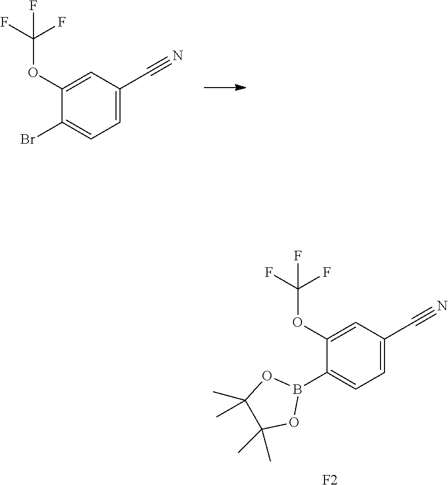

4-Bromo-3-(trifluoromethoxy)benzonitrile (7.50 g, 28.2 mmol) is dissolved in dioxane (120 mL). Bis(pinacolato)diboron (10.7 g, 42.3 mmol) and potassium acetate (6.91 g, 70.5 mmol) are added, and the mixture is purged with argon. Pd(dppf)Cl₂×DCM (2.30 g, 2.82 mmol) is added, and the reaction mixture is stirred at 80° C. for 2 h. After the reaction mixture is cooled to RT it is diluted with ACN/MeOH 5/1 and is filtered through a pad of celite and SiO₂. The filtrated is concentrated and the residue is purified by flash chromatography (CycH/EtOAc 100/0→CycH/EtOAc 75/25→CycH/EtOAc 0/100) to afford the intermediate F2.

Analysis (method C): Rₜ: 0.61 min and 1.14 min (boronic ester)

TLC: silica gel, CycH/EtOAc 4/1: R_{f}: 0.4-0.5

### Synthesis of Intermediate F3

### Synthesis of [2-(2,2-difluoroethoxy)-4-(methoxycarbonyl)phenyl]boronic acid (Intermediate F3)

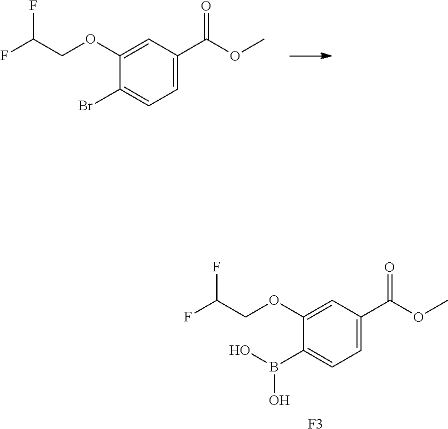

Methyl 4-bromo-3-(2,2-difluoroethoxy)benzoate (300 mg, 0.97 mmol, 95% purity) is dissolved in dioxane (5 mL). Bis(neopentyl glycolato)diboron (240 mg, 1.06 mmol) and potassium acetate (303 mg, 3.09 mmol) are added, and the mixture is purged with argon. Pd(dppf)Cl₂×DCM (78.9 mg, 0.10 mmol) is added, and the reaction mixture is stirred at 100° C. for 2 h. After the reaction mixture is cooled to RT, it is diluted with ACN, filtered, and purified by reversed phase chromatography (HPLC; ACN/water/TFA) to obtain the intermediate F3.

Analysis (method E): Rₜ: 0.78 min. [M+H]⁺: 261

### Synthesis of Intermediate F4

### Synthesis of 2-{4-[(S)-ethyl(methyl)phosphoryl]-2-(trifluoromethoxy)phenyl}-4,4,5,5-tetramethyl-1,3,2-dioxaborolane (Intermediate F4)

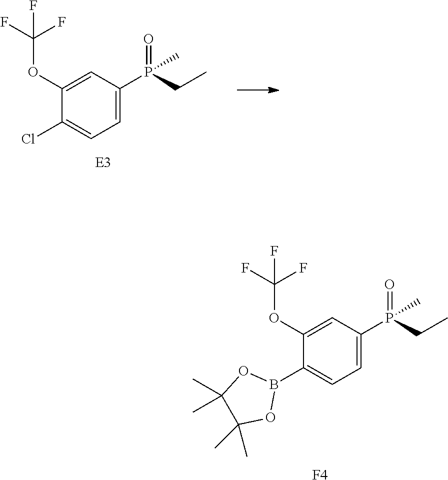 [SMILES: CC[P@](C)(=O)c1ccc(Cl)c(OC(F)(F)F)c1 ; CC[P@](C)(=O)c1ccc(B2OC(C)(C)C(C)(C)O2)c(OC(F)(F)F)c1 ; "F4"]

1-Chloro-4-[ethyl(methyl)phosphoryl]-2-(trifluoromethoxy)benzene (E3) (100 mg, 0.35 mmol) is dissolved in dioxane (3 mL). Bis(pinacolato)diboron (177 mg, 0.70 mmol) and potassium acetate (105 mg, 1.07 mmol) are added, and the mixture is purged with argon. Xphos Pd G3 (20.0 mg, 0.02 mmol) is added, and the reaction mixture is stirred at 80° C. for 40 min and at 100° C. for 1 h. After the reaction mixture is cooled to RT it is filtered and the filtrate is concentrated to afford the intermediate F4.

Analysis (method C): Rₜ: 0.90 min, [M+H]⁺: 379

The intermediates compiled in the following table are obtained by following a reaction sequence analogous to that described for intermediate F3.

| | | | | MS | |
|---|---|---|---|---|---|
| | | | | (ESI+): | |
| Inter- | Starting materials | | LC | m/z | t_{R} |
| mediate | and conditions | Structure/Name | Method | [M + H]⁺ | [min] |
| F5 | E4 + Bis(neopentyl glycolato)diboron KOAc Pd(dppf)Cl₂ × DCM 90° C. 26 h | 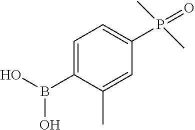 [4-(dimethylphosphoryl)-2- methylphenyl]boronic acid | D | 213 | 0.12 |
| F6 | E5 + Bis(neopentyl glycolato)diboron KOAc Pd(dppf)Cl₂ × DCM 100° C. 2 h | 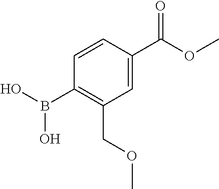 [SMILES: COCc1cc(C(=O)OC)ccc1B(O)O] [4-(methoxycarbonyl)-2- (methoxymethyl)phenyl]boronic acid | E | 247 (+ Na) | 0.69 |
| F7 | E2 + Bis(neopentyl glycolato)diboron KOAc Pd(dppf)Cl₂ × DCM 100° C. 2 h | 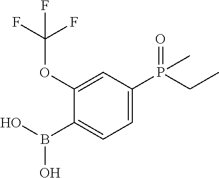 [SMILES: CCP(C)(=O)c1ccc(B(O)O)c(OC(F)(F)F)c1] {4-[ethyl(methyl)phosphoryl]-2- (trifluoromethoxy)phenyl}boronic acid | E | 297 | 0.62 |
| F8 | E6 + Bis(neopentyl glycolato)diboron KOAc Bis[cinnamyl palladium(II) chloride] Rac-BIDIME 90° C. 2.5 h | 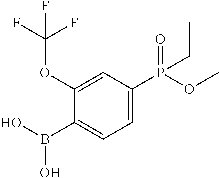 [SMILES: CCP(=O)(OC)c1ccc(B(O)O)c(OC(F)(F)F)c1] [4-(diethylphosphoryl)-2- (trifluoromethoxy)phenyl]boronic acid | A | 311 | 0.34 |

### Synthesis of Intermediate F9

### Synthesis of [4-(dimethylphosphoryl)-2-(trifluoromethoxy)phenyl]boronic acid (Intermediate F9)

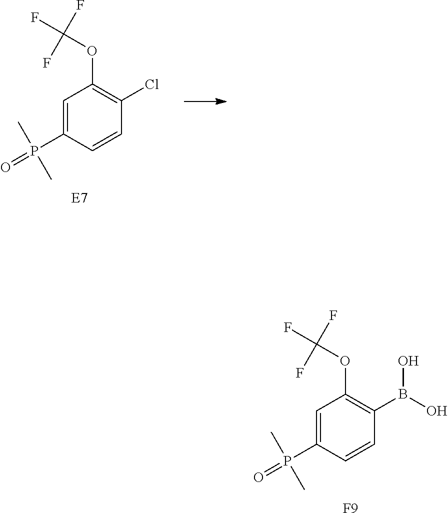

1-Chloro-4-(dimethylphosphoryl)-2-(trifluoromethoxy)benzene (E7) (613 mg, 2.24 mmol), Bis(neopentyl glycolato)diboron (1.27 g, 5.62 mmol), Pd(dppf)Cl₂×DCM (300 mg, 0.367 mmol) and potassium acetate (662 mg, 6.74 mmol) are dissolved in dioxane (10 mL). The mixture is purged with argon and is stirred at 100° C. for 3 h. After the reaction mixture is cooled to RT, filtered and the filtrate is concentrated. To the residue is added ACN (10 ml), water (2 ml) and TFA (0.5 ml) and the precipitation is filtered. The filtrate is purified by reversed phase chromatography (HPLC; ACN/water/TFA) to obtain the intermediate F9.

Analysis (method C): Rₜ: 0.37 min, [M+H]⁺: 283

### Synthesis of Intermediates G1-G10

### Synthesis of Intermediate G1

### Synthesis of 4-(4-bromo-5-cyclopropyl-1-methyl-1H-imidazol-2-yl)-N-methyl-3-(trifluoromethoxy)benzamide (Intermediate G1)

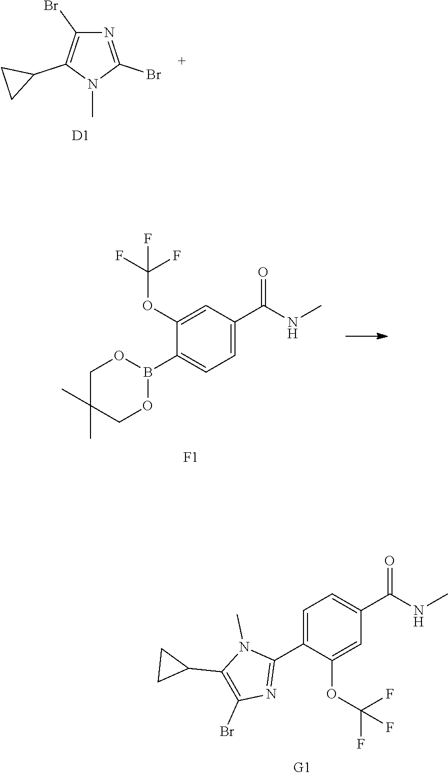

2,4-Dibromo-5-cyclopropyl-1-methyl-1H-imidazole (D1) (0.80 g, 2.86 mmol) is dissolved in dioxane (12 mL) and water (3 mL). 4-(5,5-dimethyl-1,3,2-dioxaborinan-2-yl)-N-methyl-3-(trifluoromethoxy) benzamide (F1) (2.00 g, 2.96 mmol, 49% purity) and an aq. solution of Cs₂CO₃ (1 M, 9.00 mL, 9.00 mmol) are added, and the mixture is purged with argon. Pd(PPh₃)₄ (0.33 g, 0.29 mmol) is added, and the reaction mixture is stirred at 80° C. for 3 h. After the reaction mixture is cooled to RT, it is diluted with isopropyl acetate (20 mL). The organic layer is separated and concentrated. The residue is purified by preparative HPLC (HPLC; ACN/water/NH₃) to afford intermediate Gh.

Analysis (method C: Rₜ: 0.76 min, [M+H]⁺: 418

The intermediates compiled in the following table are obtained by following a reaction sequence analogous to that described for intermediate G1.

| | | | | MS | |
|---|---|---|---|---|---|
| | | | | (ESI+): | |
| Inter- | Starting materials | | LC | m/z | t_{R} |
| mediate | and conditions | Structure/Name | Method | [M + H]⁺ | [min] |
| G2 | D1 + F2 Xphos Pd G3 80° C., 2 h | 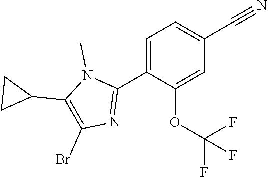 [SMILES: Cn1c(-c2ccc(C#N)cc2OC(F)(F)F)nc(Br)c1C1CC1] 4-(4-bromo-5-cyclopropyl-1-methyl- 1H-imidazol-2-yl)-3- (trifluoromethoxy)benzonitrile | A | 386 | 0.63 |
| G3 | D1 + F3 Xphos Pd G3 80° C., 2 h | 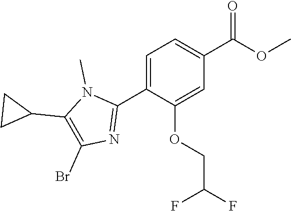 [SMILES: COC(=O)c1ccc(-c2nc(Br)c(C3CC3)n2C)c(OCC(F)F)c1] methyl 4-(4-bromo-5-cyclopropyl-1- methyl-1H-imidazol-2-yl)-3-(2,2- difluoroethoxy)benzoate | B | 416 | 0.96 |
| G6 | D1 + F6 Xphos Pd G3 80° C., 2 h | 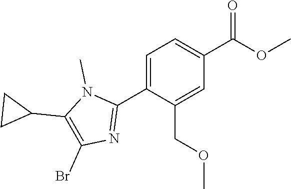 [SMILES: COCc1cc(C(=O)OC)ccc1-c1nc(Br)c(C2CC2)n1C] methyl 4-(4-bromo-5-cyclopropyl-1- methyl-1H-imidazol-2-yl)-3- (methoxymethyl)benzoate | E | 379 | 0.87 |
| G7 | D1 + F7 Xphos Pd G3 80° C., 2 h | 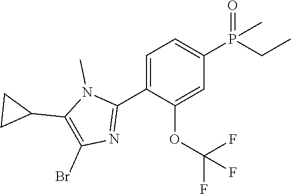 [SMILES: CCP(C)(=O)c1ccc(-c2nc(Br)c(C3CC3)n2C)c(OC(F)(F)F)c1] 4-bromo-5-cyclopropyl-2-{4- [ethyl(methyl)phosphoryl]-2- (trifluoromethoxy)phenyl}-1-methyl- 1H-imidazole | E | 451 | 0.87 |
| G9 | D1 + F8 Pd(PPh₃)₄ DMF 85° C., 2 h | 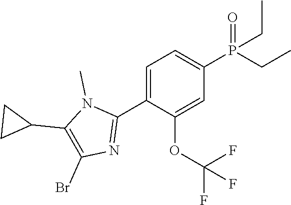 [SMILES: CCP(=O)(CC)c1ccc(-c2nc(Br)c(C3CC3)n2C)c(OC(F)(F)F)c1] 4-bromo-5-cyclopropyl-2-[4- (diethylphosphoryl)-2- (trifluoromethoxy)phenyl]-1-methyl- 1H-imidazole | A | 465 | 0.56 |

### Synthesis of Intermediate G10

### Synthesis of 4-bromo-5-cyclopropyl-2-[4-(dimethylphosphoryl)-2-(trifluoromethoxy)phenyl]-1-methyl-1H-imidazole (Intermediate G10)

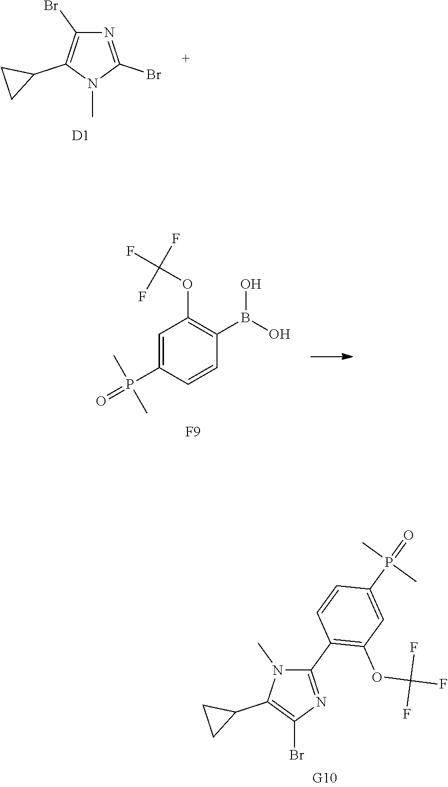

2,4-Dibromo-5-cyclopropyl-1-methyl-1H-imidazole (D1) (360 mg, 1.28 mmol), [4-(dimethylphosphoryl)-2-(trifluoromethoxy)phenyl]boronic acid (400 mg, 0.851 mmol), Xphos Pd G3 (75.0 mg, 0.0868 mmol) and an aq. solution of Cs₂CO₃ (1 M, 2.55 mL, 2.55 mmol) are dissolved in dioxane (5 mL). The reaction mixture is stirred at 80° C. for 3 h and after at RT for 14 h. The reaction mixture is extracted with DCM and water. The organic layer is separated and concentrated. The residue is purified by preparative HPLC (HPLC; ACN/water/TFA) to afford intermediate G10.

Analysis (method C): Rₜ: 0.70 min, [M+H]⁺: 437

### Synthesis of Intermediate G4

### Synthesis of 4-bromo-5-cyclopropyl-2-{4-[(S)-ethyl(methyl)phosphoryl]-2-(trifluoromethoxy) phenyl}-1-methyl-1H-imidazole (Intermediate G4)

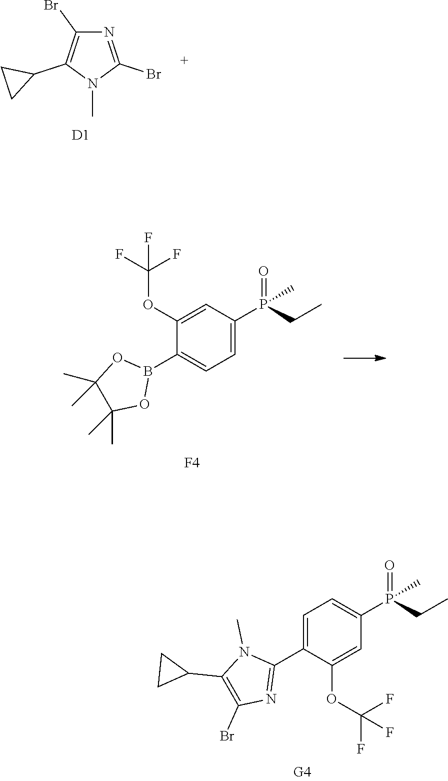

2,4-Dibromo-5-cyclopropyl-1-methyl-1H-imidazole (D1) (98.0 mg, 0.35 mmol) is dissolved in dioxane (3 mL). 2-{4-[Ethyl(methyl)phosphoryl]-2-(trifluoromethoxy)phenyl}-4,4,5,5-tetramethyl-1,3,2-dioxaborolane (F4) (0.30 g, 0.35 mmol, 44% purity) and an aq. solution of Cs₂CO₃ (1 M, 1.40 mL, 1.40 mmol) are added, and the mixture is purged with argon. Xphos Pd G3 (30.0 mg, 0.04 mmol) is added, and the reaction mixture is stirred at 80° C. for 2 h. After the reaction mixture is cooled to RT, it is diluted with DCM (20 mL) and water (10 mL). The organic layer is separated and concentrated. The residue is purified by preparative HPLC (HPLC; ACN/water/TFA) to afford intermediate G4.

Analysis (method C): Rₜ: 0.75 min, [M+H]⁺: 451

### Synthesis of Intermediate G5

### Synthesis of 1-[4-(dimethylphosphoryl)-2-methylphenyl]-3-iodo-5-methyl-4-(propan-2-yl)-1H-pyrazole (Intermediate G5)

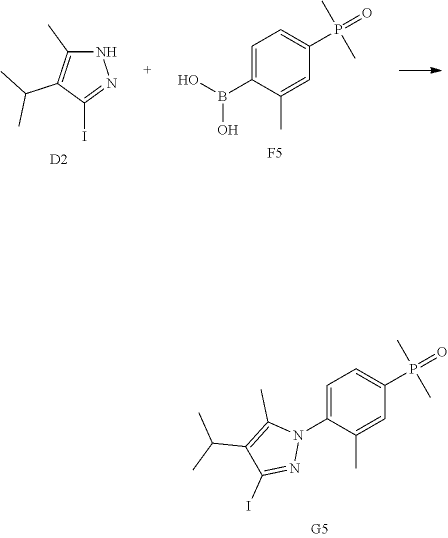

3-Iodo-5-methyl-4-(propan-2-yl)-1H-pyrazole (D2) (280 mg, 1.12 mmol), [4-(dimethylphosphoryl)-2-methylphenyl]boronic acid (F5) (261 mg, 1.23 mmol) and copper (II) acetate (61.0 mg, 0.34 mmol) are dissolved in ACN (10 mL), DCM (10 mL) and pyridine (159 mg, 2.02 mmol). The reaction mixture is stirred at 60° C. for 14 h under an atmosphere of air. The reaction mixture is filtered, and the filtrate is purified by preparative HPLC (HPLC; ACN/water/TFA) to afford intermediate G5.

Analysis (method E): Rₜ: 1.02 min, [M+H]⁺: 417

### Synthesis of Intermediate G8

### Step 1: Synthesis of 4-(4-bromo-5-cyclopropyl-1-methyl-1H-imidazol-2-yl)-3-(trifluoromethoxy)benzoic acid

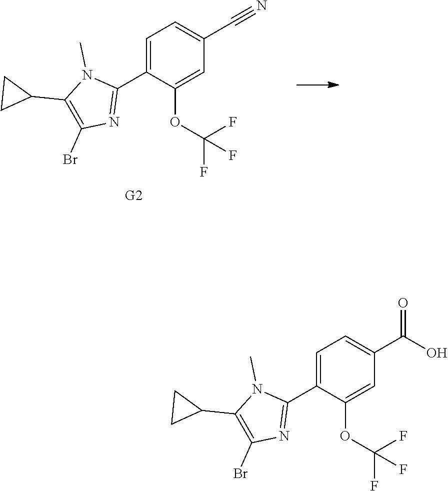 [SMILES: Cn1c(-c2ccc(C#N)cc2OC(F)(F)F)nc(Br)c1C1CC1 ; Cn1c(-c2ccc(C(=O)O)cc2OC(F)(F)F)nc(Br)c1C1CC1]

4-(4-Bromo-5-cyclopropyl-1-methyl-1H-imidazol-2-yl)-3-(trifluoromethoxy)benzonitrile (G2) (0.20 g, 0.52 mmol) is dissolve in EtOH (5 mL). NaOH (4 M, 1.50 mL, 6.00 mmol) is added, and the reaction mixture is stirred at 80° C. for 1.5 h. After the reaction mixture is cooled to RT, it is acidified with TFA (1 mL) and purified by preparative HPLC (HPLC; ACN/water/TFA) to afford the desired compound.

Analysis (method C): Rₜ: 0.75 min, [M+H]⁺: 405

### Step 2: Synthesis of 4-(4-bromo-5-cyclopropyl-1-methyl-1H-imidazol-2-yl)-N,N-dimethyl-3-(trifluoromethoxy)benzamide (Intermediate G8)

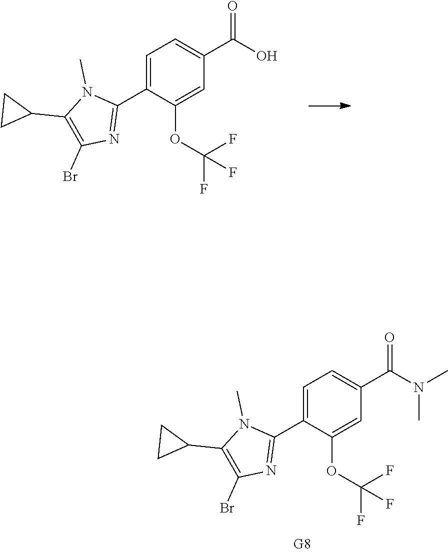 [SMILES: Cn1c(-c2ccc(C(=O)O)cc2OC(F)(F)F)nc(Br)c1C1CC1 ; CN(C)C(=O)c1ccc(-c2nc(Br)c(C3CC3)n2C)c(OC(F)(F)F)c1]

4-(4-Bromo-5-cyclopropyl-1-methyl-1H-imidazol-2-yl)-3-(trifluoromethoxy)benzoic acid×TFA (0.17 g, 0.32 mmol) is dissolved in DMF (3 mL). DIPEA (0.28 mL, 1.65 mmol) and HATU (0.19 g, 0.49 mmol) are added and stirred at rt for 5 min. Then dimethyl amine (2 M in THF, 0.41 mL, 0.81 mmol) is added and the reaction mixture is stirred at rt for 20 min. The reaction mixture is purified by preparative HPLC (HPLC; ACN/water/NH₃) to afford the intermediate G8.

Analysis (method C): Rₜ: 0.78 min, [M+H]⁺: 432

### Synthesis of Examples

### Synthesis of Examples 1, 2, 9, 15, 17, 18 and 19

### Synthesis of 4-{5-cyclopropyl-4-[2-(1-cyclopropyl-3-methoxy-1H-pyrazol-4-yl)-2H-indazol-4-yl]-1-methyl-1H-imidazol-2-yl}-N-methyl-3-(trifluoromethoxy)benzamide (Example 1)

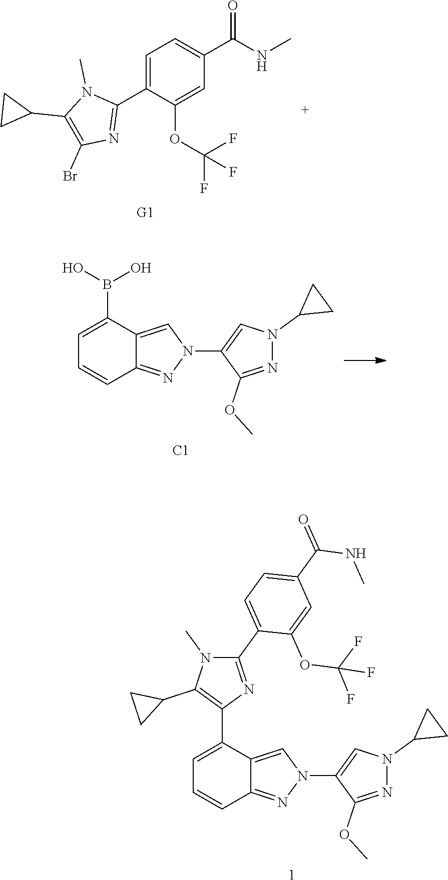 [SMILES: CNC(=O)c1ccc(-c2nc(Br)c(C3CC3)n2C)c(OC(F)(F)F)c1 ; COc1nn(C2CC2)cc1-n1cc2c(B(O)O)cccc2n1 ; "C1" ; CNC(=O)c1ccc(-c2nc(-c3cccc4nn(-c5cn(C6CC6)nc5OC)cc34)c(C3CC3)n2C)c(OC(F)(F)F)c1 ; "1"]

4-(4-Bromo-5-cyclopropyl-1-methyl-1H-imidazol-2-yl)-N-methyl-3-(trifluoromethoxy) benzamide (G1) (63.1 mg, 0.15 mmol) is dissolved in dioxane (2 mL). [2-(1-Cyclopropyl-3-methoxy-1H-pyrazol-4-yl)-2H-indazol-4-yl]boronic acid (C1) (45.0 mg, 0.15 mmol) and an aqueous solution of K₃PO₄ (2 M, 604 μL, 1.21 mmol) are added, and the mixture is purged with argon. XPhos Pd G3 (6.39 mg, 0.01 mmol) is added, and the reaction mixture is stirred at 80° C. for 3 h. After the reaction mixture is cooled to RT, it is filtered, and the residue is purified by preparative HPLC (HPLC; ACN/water/TFA) to afford example 1.

Analysis (method C) Rₜ: 0.67 min, [M+H]⁺: 592

The examples compiled in the following table are obtained by following a reaction sequence analogous to that described for example 1.

| | | | | MS | |
|---|---|---|---|---|---|
| | Starting | | | (ESI+): | |
| Exam- | materials and | | LC | m/z | t_{R} |
| ple | conditions | Structure/Name | Method | [M + H]⁺ | [min] |
| 2 | G10 + C9 XPhos Pd G3 K₃PO₄ 80° C., 3 h | 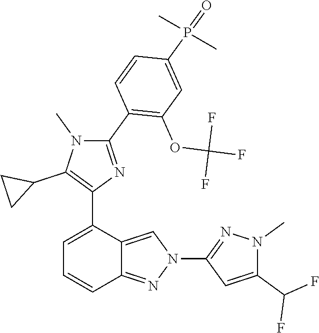 [SMILES: Cn1nc(-n2cc3c(-c4nc(-c5ccc(P(C)(C)=O)cc5OC(F)(F)F)n(C)c4C4CC4)cccc3n2)cc1C(F)F] 4-{5-cyclopropyl-2-[4- (dimethylphosphoryl)-2- (trifluoromethoxy)phenyl]-1-methyl- 1H-imidazol-4-yl}-2-[5- (difluoromethyl)-1-methyl-1H- pyrazol-3-yl]-2H-indazole | C | 605 | 0.64 |
| 9 | G5 + C1 XPhos Pd G3 K₃PO₄ 80° C., 3 h | 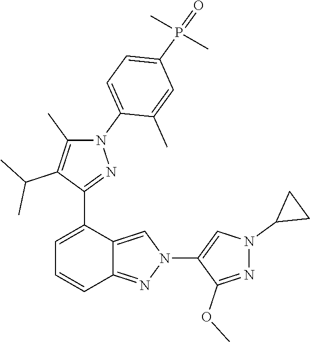 [SMILES: COc1nn(C2CC2)cc1-n1cc2c(-c3nn(-c4ccc(P(C)(C)=O)cc4C)c(C)c3C(C)C)cccc2n1] 2-(1-cyclopropyl-3-methoxy-1H- pyrazol-4-yl)-4-{1-[4- (dimethylphosphoryl)-2- methylphenyl]-5-methyl-4-(propan-2- yl)-1H-pyrazol-3-yl}-2H-indazole | A | 543 | 0.65 |
| 15 | G8 + C1 XPhos Pd G3 K₃PO₄ 80° C., 3 h | 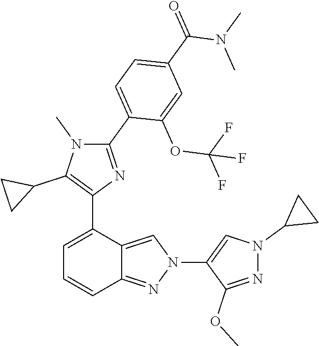 [SMILES: COc1nn(C2CC2)cc1-n1cc2c(-c3nc(-c4ccc(C(=O)N(C)C)cc4OC(F)(F)F)n(C)c3C3CC3)cccc2n1] 4-{5-cyclopropyl-4-[2-(1-cyclopropyl- 3-methoxy-1H-pyrazol-4-yl)-2H- indazol-4-yl]-1-methyl-1H-imidazol-2- yl}-N,N-dimethyl-3- (trifluoromethoxy)benzamide | A | 606 | 0.55 |
| 17 | G9 + C7 XPhos Pd G3 K₃PO₄ 80° C., 3 h | 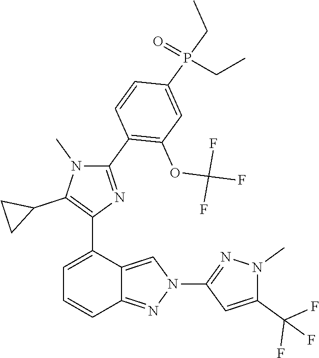 [SMILES: CCP(=O)(CC)c1ccc(-c2nc(-c3cccc4nn(-c5cc(C(F)(F)F)n(C)n5)cc34)c(C3CC3)n2C)c(OC(F)(F)F)c1] 4-{5-cyclopropyl-2-[4- (diethylphosphoryl)-2- (trifluoromethoxy)phenyl]-1-methyl- 1H-imidazol-4-yl}-2-[1-methyl-5- (trifluoromethyl)-1H-pyrazol-3-yl]-2H- indazole | D | 651 | 0.79 |
| 18 | G4 + C10 XPhos Pd G3 K₃PO₄ 80° C., 2 h | 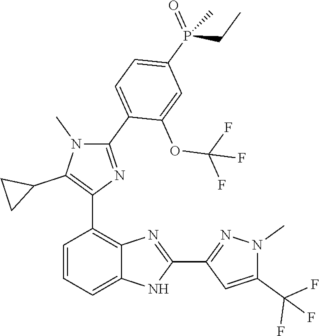 [SMILES: CC[P@](C)(=O)c1ccc(-c2nc(-c3cccc4[nH]c(-c5cc(C(F)(F)F)n(C)n5)nc34)c(C3CC3)n2C)c(OC(F)(F)F)c1] 4-(5-cyclopropyl-2-{4-[(S)- ethyl(methyl)phosphoryl]-2- (trifluoromethoxy)phenyl}-1-methyl- 1H-imidazol-4-yl)-2-[1-methyl-5- (trifluoromethyl)-1H-pyrazol-3-yl]-1H- 1,3-benzodiazole | J | 637 | 0.90 |
| 19 | G4 + C11 XPhos Pd G3 K₃PO₄ 80° C., 2 h | 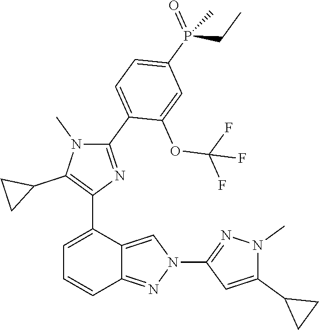 [SMILES: CC[P@](C)(=O)c1ccc(-c2nc(-c3cccc4nn(-c5cc(C6CC6)n(C)n5)cc34)c(C3CC3)n2C)c(OC(F)(F)F)c1] 2-(5-cyclopropyl-1-methyl-1H-pyrazol- 3-yl)-4-(5-cyclopropyl-2-{4-[(S)- ethyl(methyl)phosphoryl]-2- (trifluoromethoxy)phenyl}-1-methyl- 1H-imidazol-4-yl)-2H-indazole | A | 609 | 0.54 |

### Synthesis of Examples 4, 8, 11 and 16

### Synthesis of Example 4

### Step 1: Synthesis of 4-{5-cyclopropyl-4-[2-(3-ethoxy-1-methyl-1H-pyrazol-4-yl)-2H-indazol-4-yl]-1-methyl-1H-imidazol-2-yl}-3-(trifluoromethoxy)benzonitrile

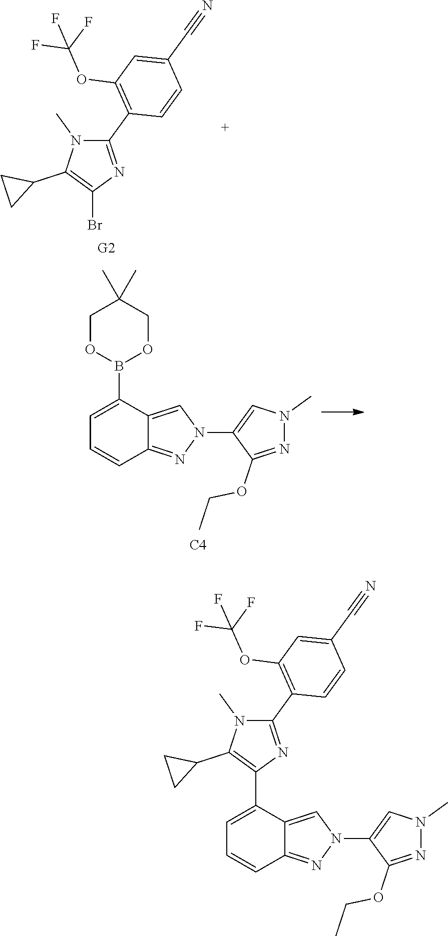

4-(4-Bromo-5-cyclopropyl-1-methyl-1H-imidazol-2-yl)-3-(trifluoromethoxy)benzonitrile (G2) (59.1 mg, 0.153 mmol) is dissolved in dioxane (1 mL). 4-(5,5-dimethyl-1,3,2-dioxaborinan-2-yl)-2-(3-ethoxy-1-methyl-1H-pyrazol-4-yl)-2H-indazole (C4) (86.0 mg, 0.153 mmol) and an aqueous solution of K₃PO₄ (2 M, 382 μL, 0.765 mmol) are added, and the mixture is purged with argon. XPhos Pd G3 (20.0 mg, 0.0236 mmol) is added, and the reaction mixture is stirred at 80° C. for 2 h. After the reaction mixture is cooled to RT, diluted with DCM and water. The organic phase is separated and concentrated to afford the desired compound.

Analysis (method C): Rₜ: 0.73 min, [M+H]⁺: 548

### Step 2: Synthesis 4-{5-cyclopropyl-4-[2-(3-ethoxy-1-methyl-1H-pyrazol-4-yl)-2H-indazol-4-yl]-1-methyl-1H-imidazol-2-yl}-3-(trifluoromethoxy)benzamide (Example 4)

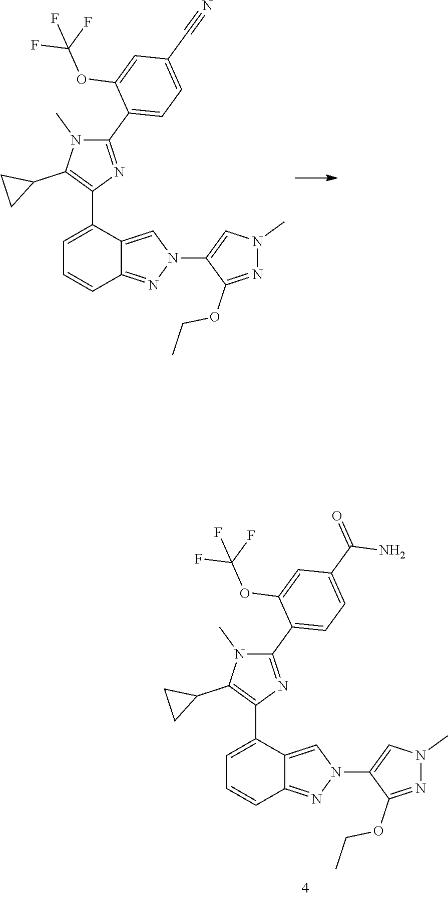 [SMILES: CCOc1nn(C)cc1-n1cc2c(-c3nc(-c4ccc(C#N)cc4OC(F)(F)F)n(C)c3C3CC3)cccc2n1 ; CCOc1nn(C)cc1-n1cc2c(-c3nc(-c4ccc(C(N)=O)cc4OC(F)(F)F)n(C)c3C3CC3)cccc2n1]

4-{5-cyclopropyl-4-[2-(3-ethoxy-1-methyl-1H-pyrazol-4-yl)-2H-indazol-4-yl]-1-methyl-1H-imidazol-2-yl}-3-(trifluoromethoxy)benzonitrile (135 mg, 0.153 mmol) is dissolved in DMSO (1.5 mL). H₂O₂ (30%, 394 μL, 3.82 mmol) and K₂CO₃ (71.0 mg, 0.514 mmol) are added, and the reaction mixture is stirred at rt for 30 min. The reaction mixture is filtered, and the residue is purified by preparative HPLC (HPLC; ACN/water/TFA) to afford example 4.

Analysis (method C): Rₜ: 0.63 min, [M+H]⁺: 566

The examples compiled in the following table are obtained by following a reaction sequence analogous to that described for example 4.

| | | | | MS | |
|---|---|---|---|---|---|
| | Starting | | | (ESI+): | |
| Exam- | materials and | | LC | m/z | t_{R} |
| ple | conditions | Structure/Name | Method | [M + H]⁺ | [min] |
| 8 | G2 + C6 XPhos Pd G3 K₃PO₄ 95° C., 2 h H₂O₂, K₂CO₃ rt, 2 h | 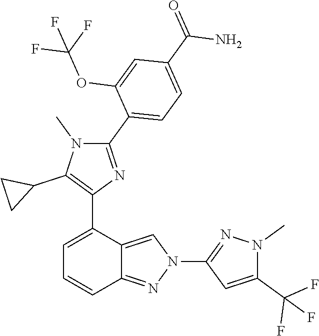 [SMILES: Cn1nc(-n2cc3c(-c4nc(-c5ccc(C(N)=O)cc5OC(F)(F)F)n(C)c4C4CC4)cccc3n2)cc1C(F)(F)F] 4-(5-cyclopropyl-1-methyl-4-{2-[1- methyl-5-(trifluoromethyl)-1H- pyrazol-3-yl]-2H-indazol-4-yl}-1H- imidazol-2-yl)-3- (trifluoromethoxy)benzamide | J | 590 | 0.97 |
| 11 | G2 + C8 XPhos Pd G3 K₃PO₄ 95° C., 3 h H₂O₂, K₂CO₃ rt, 2 h | 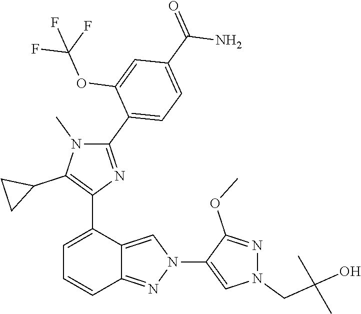 [SMILES: COc1nn(CC(C)(C)O)cc1-n1cc2c(-c3nc(-c4ccc(C(N)=O)cc4OC(F)(F)F)n(C)c3C3CC3)cccc2n1] 4-(5-cyclopropyl-4-{2-[1-(2-hydroxy- 2-methylpropyl)-3-methoxy-1H- pyrazol-4-yl]-2H-indazol-4-yl}-1- methyl-1H-imidazol-2-yl)-3- (trifluoromethoxy)benzamide | E | 610 | 0.85 |
| 16 | G2 + C1 XPhos Pd G3 K₃PO₄ 80° C., 3 h H₂O₂, K₂CO₃ rt, 2 h | 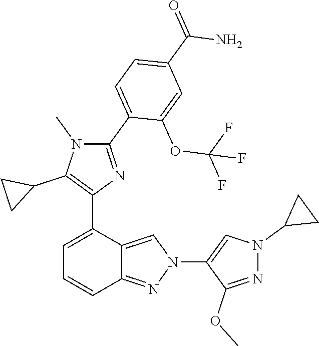 [SMILES: COc1nn(C2CC2)cc1-n1cc2c(-c3nc(-c4ccc(C(N)=O)cc4OC(F)(F)F)n(C)c3C3CC3)cccc2n1] 4-{5-cyclopropyl-4-[2-(1-cyclopropyl- 3-methoxy-1H-pyrazol-4-yl)-2H- indazol-4-yl]-1-methyl-1H-imidazol- 2-yl}-3-(trifluoromethoxy)benzamide | A | 578 | 0.51 |

### Synthesis of Examples 3 and 5

### Synthesis of Example 3

### Step 1: Synthesis of 4-{5-cyclopropyl-4-[2-(3-methoxy-1-methyl-1H-pyrazol-4-yl)-2H-indazol-4-yl]-1-methyl-1H-imidazol-2-yl}-3-(trifluoromethoxy)benzonitrile

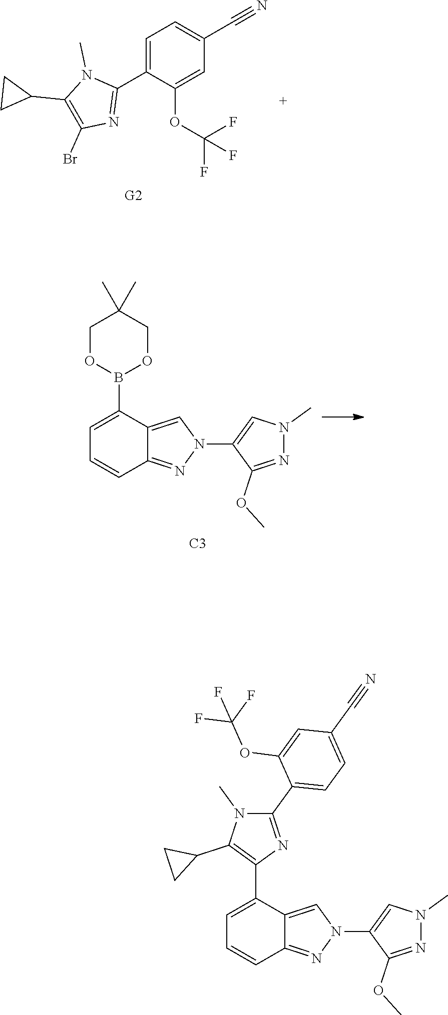 [SMILES: Cn1c(-c2ccc(C#N)cc2OC(F)(F)F)nc(Br)c1C1CC1 ; COc1nn(C)cc1-n1cc2c(B3OCC(C)(C)CO3)cccc2n1 ; "C3" ; COc1nn(C)cc1-n1cc2c(-c3nc(-c4ccc(C#N)cc4OC(F)(F)F)n(C)c3C3CC3)cccc2n1]

4-(4-Bromo-5-cyclopropyl-1-methyl-1H-imidazol-2-yl)-3-(trifluoromethoxy)benzonitrile (G2) (0.27 g, 0.70 mmol) is dissolved in dioxane (5 mL). 4-(5,5-Dimethyl-1,3,2-dioxaborinan-2-yl)-2-(3-methoxy-1-methyl-1H-pyrazol-4-yl)-2H-indazole (C₃) (0.30 g, 0.75 mmol, 85% purity) and an aqueous solution of K₃PO₄ (2 M, 1.05 mL, 2.10 mmol) are added, and the mixture is purged with argon. XPhos Pd G3 (59.2 mg, 0.07 mmol) is added, and the reaction mixture is stirred at 80° C. for 2 h. After the reaction mixture is cooled to RT, it is diluted with ACN (4 mL), filtered, and concentrated. The residue is purified by preparative HPLC (HPLC; ACN/water/TFA) to afford the desired compound.

Analysis (method C): Rₜ: 0.68 min, [M+H]⁺: 534

### Step 2: Synthesis of 4-{5-cyclopropyl-4-[2-(3-methoxy-1-methyl-1H-pyrazol-4-yl)-2H-indazol-4-yl]-1-methyl-1H-imidazol-2-yl}-3-(trifluoromethoxy)benzoic acid

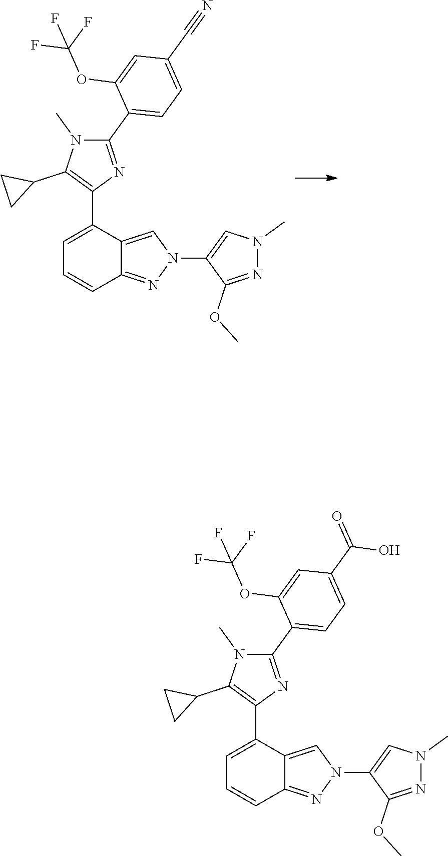 [SMILES: COc1nn(C)cc1-n1cc2c(-c3nc(-c4ccc(C#N)cc4OC(F)(F)F)n(C)c3C3CC3)cccc2n1 ; COc1nn(C)cc1-n1cc2c(-c3nc(-c4ccc(C(=O)O)cc4OC(F)(F)F)n(C)c3C3CC3)cccc2n1]

4-{5-Cyclopropyl-4-[2-(3-methoxy-1-methyl-1H-pyrazol-4-yl)-2H-indazol-4-yl]-1-methyl-1H-imidazol-2-yl}-3-(trifluoromethoxy)benzonitrile×TFA (0.14 g, 0.22 mmol) is dissolved in EtOH (2 mL). NaOH (4 M, 0.55 mL, 2.20 mmol) is added, and the reaction mixture is stirred at 80° C. for 1 h and at rt overnight. The reaction mixture is diluted with water (10 mL) and acidified with 4 M HCl (0.55 mL) till pH 4. The formed precipitate is filtered, washed with water, and dried in vacuum at 40° C. to afford the desired compound.

Analysis (method C): Rₜ: 0.64 min, [M+H]⁺: 553

### Step 3: Synthesis of 4-{5-cyclopropyl-4-[2-(3-methoxy-1-methyl-1H-pyrazol-4-yl)-2H-indazol-4-yl]-1-methyl-1H-imidazol-2-yl}-N,N-dimethyl-3-(trifluoromethoxy)benzamide (Example 3)

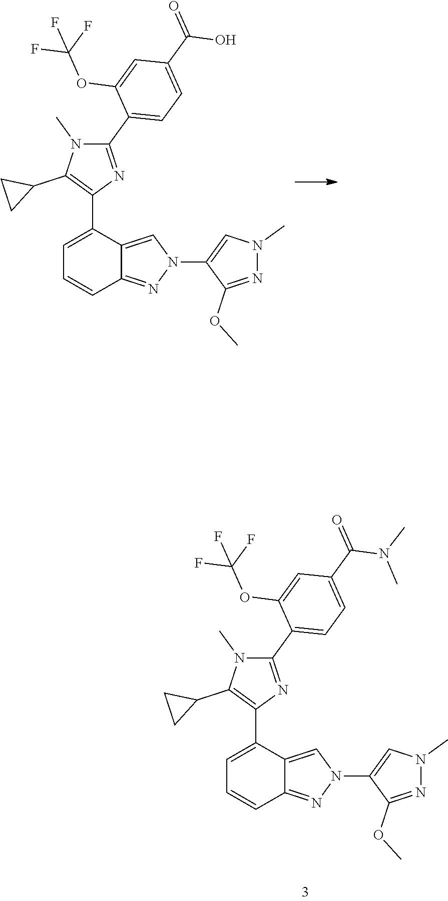 [SMILES: COc1nn(C)cc1-n1cc2c(-c3nc(-c4ccc(C(=O)O)cc4OC(F)(F)F)n(C)c3C3CC3)cccc2n1 ; COc1nn(C)cc1-n1cc2c(-c3nc(-c4ccc(C(=O)N(C)C)cc4OC(F)(F)F)n(C)c3C3CC3)cccc2n1]

4-{5-Cyclopropyl-4-[2-(3-methoxy-1-methyl-1H-pyrazol-4-yl)-2H-indazol-4-yl]-1-methyl-1H-imidazol-2-yl}-3-(trifluoromethoxy)benzoic acid×HCl (60.0 mg, 0.09 mmol, 91% purity) is dissolved in DMF (1 mL). DIPEA (48.1 μL, 0.28 mmol) and HATU (38.8 mg, 0.10 mmol) are added and stirred at rt for 10 min. Then dimethyl amine (2 M in THF, 92.7 μL, 0.19 mmol) is added and the reaction mixture is stirred at rt overnight. The reaction mixture is filtered and purified by SFC (preparative SFC; BEH_2-EP; MeOH/CO₂; 40° C.; BPR: 120 bar) to afford example 3.

Analysis (method I): Rₜ: 0.85 min, [M+H]⁺: 580

The examples compiled in the following table are obtained by following a reaction sequence analogous to that described for example 3.

| | | | | MS | |
|---|---|---|---|---|---|
| | Starting | | | (ESI+): | |
| Exam- | materials and | | LC | m/z | t_{R} |
| ple | conditions | Structure/Name | Method | [M + H]⁺ | [min] |
| 5 | G2 + C3 XPhos Pd G3 K₃PO₄ 80° C., 2 h 4 M NaOH 80° C., 1 h Methyl amine Rt, 18 h | 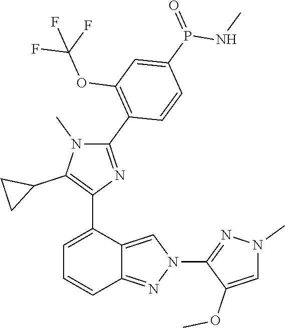 4-{5-cyclopropyl-4-[2-(3-methoxy-1- methyl-1H-pyrazol-4-yl)-2H-indazol- 4-yl]-1-methyl-1H-imidazol-2-yl}-3- (trifluoromethoxy)benzamide | C | 566 | 0.63 |

### Synthesis of Example 6 and 12

### Synthesis of Example 6

### Step 1: Synthesis of methyl 4-{5-cyclopropyl-4-[2-(1-cyclopropyl-3-methoxy-1H-pyrazol-4-yl)-2H-indazol-4-yl]-1-methyl-1H-imidazol-2-yl}-3-(2,2-difluoroethoxy)benzoate

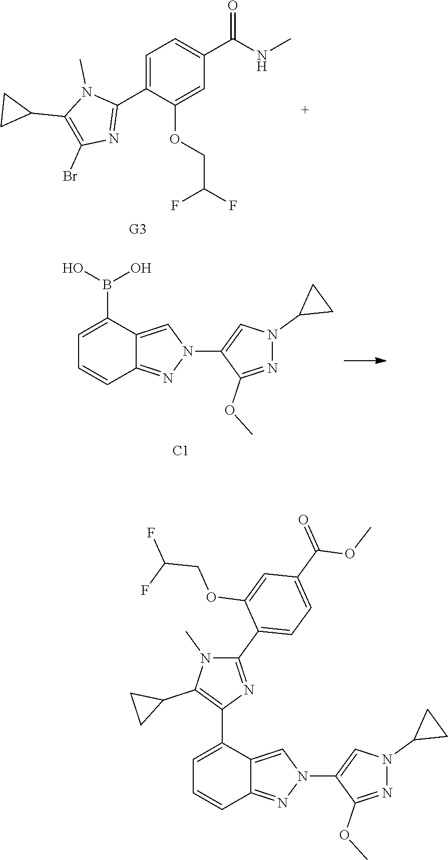 [SMILES: CNC(=O)c1ccc(-c2nc(Br)c(C3CC3)n2C)c(OCC(F)F)c1 ; COc1nn(C2CC2)cc1-n1cc2c(B(O)O)cccc2n1 ; "C1" ; COC(=O)c1ccc(-c2nc(-c3cccc4nn(-c5cn(C6CC6)nc5OC)cc34)c(C3CC3)n2C)c(OCC(F)F)c1]

Methyl 4-(4-bromo-5-cyclopropyl-1-methyl-1H-imidazol-2-yl)-3-(2,2-difluoroethoxy)benzoate (G3) (93.8 mg, 0.23 mmol) is dissolved in dioxane (5 mL). [2-(1-Cyclopropyl-3-methoxy-1H-pyrazol-4-yl)-2H-indazol-4-yl]boronic acid (C1) (80.8 mg, 0.27 mmol) and an aqueous solution of Cs₂CO₃ (2 M, 1.00 mL, 2.00 mmol) are added, and the mixture is purged with argon. Pd(dtbpf)Cl₂ (14.7 mg, 0.02 mmol) is added, and the reaction mixture is stirred at 80° C. for 5 h. After the reaction mixture is cooled to RT, it is filtered, and the residue is purified by preparative HPLC (HPLC; ACN/water/TFA) to afford the desired compound.

Analysis (method E): Rₜ: 0.83 min. [M+H]⁺: 589

### Step 2: Synthesis of 4-{5-cyclopropyl-4-[2-(1-cyclopropyl-3-methoxy-1H-pyrazol-4-yl)-2H-indazol-4-yl]-1-methyl-1H-imidazol-2-yl}-3-(2,2-difluoroethoxy)benzoic acid

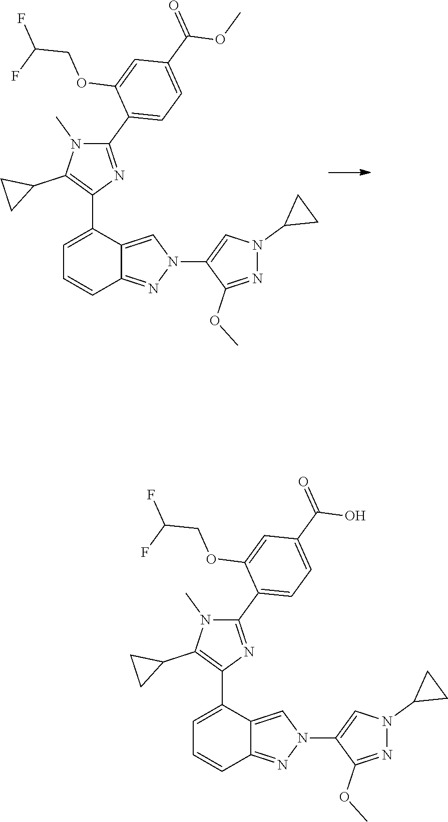 [SMILES: COC(=O)c1ccc(-c2nc(-c3cccc4nn(-c5cn(C6CC6)nc5OC)cc34)c(C3CC3)n2C)c(OCC(F)F)c1 ; COc1nn(C2CC2)cc1-n1cc2c(-c3nc(-c4ccc(C(=O)O)cc4OCC(F)F)n(C)c3C3CC3)cccc2n1]

4-{5-Cyclopropyl-4-[2-(1-cyclopropyl-3-methoxy-1H-pyrazol-4-yl)-2H-indazol-4-yl]-1-methyl-1H-imidazol-2-yl}-3-(2,2-difluoroethoxy)benzoate (117 mg, 0.20 mmol) is dissolved in MeOH (4 mL). NaOH (1 M, 500 μL, 0.50 mmol) is added, and the reaction mixture is stirred at rt overnight. The reaction mixture is neutralized with 1 M HCl and concentrated. The residue is triturated with acetone, the precipitate is filtered, and the filtrate is concentrated to afford the desired compound.

Analysis (method E): Rₜ: 0.80 min, [M+H]⁺: 575

### Step 3: Synthesis of 4-{5-cyclopropyl-4-[2-(1-cyclopropyl-3-methoxy-1H-pyrazol-4-yl)-2H-indazol-4-yl]-1-methyl-1H-imidazol-2-yl}-3-(2,2-difluoroethoxy)benzamide (Example 6)

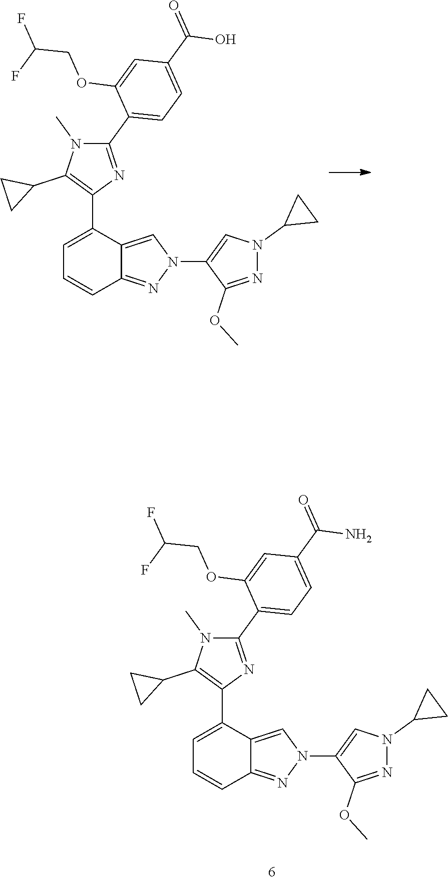 [SMILES: COc1nn(C2CC2)cc1-n1cc2c(-c3nc(-c4ccc(C(=O)O)cc4OCC(F)F)n(C)c3C3CC3)cccc2n1 ; COc1nn(C2CC2)cc1-n1cc2c(-c3nc(-c4ccc(C(N)=O)cc4OCC(F)F)n(C)c3C3CC3)cccc2n1]

4-{5-Cyclopropyl-4-[2-(1-cyclopropyl-3-methoxy-1H-pyrazol-4-yl)-2H-indazol-4-yl]-1-methyl-1H-imidazol-2-yl}-3-(2,2-difluoroethoxy)benzoic acid (40.0 mg, 0.07 mmol) is dissolved in DMF (2 mL). DIPEA (60.2 μL, 0.35 mmol), HATU (29.1 mg, 0.08 mmol) and ammonium chloride (11.2 mg, 0.21 mmol) are added, and the reaction mixture is stirred at rt overnight. The reaction mixture is filtered and purified by preparative HPLC (HPLC; ACN/water/NH₃) to afford example 6.

Analysis (method E): Rₜ: 0.78 min, [M+H]⁺: 574

The examples compiled in the following table are obtained by following a reaction sequence analogous to that described for example 6.

| | | | | MS | |
|---|---|---|---|---|---|
| | Starting | | | (ESI+): | |
| Exam- | materials and | | LC | m/z | t_{R} |
| ple | conditions | Structure/Name | Method | [M + H]⁺ | [min] |
| 12 | G6 + C1 Pd(dtbpf)Cl₂ Cs₂CO₃ 80° C., 3 h 1 M NaOH rt, 16 h ammonium carbonate HATU, TEA Rt, 18 h | 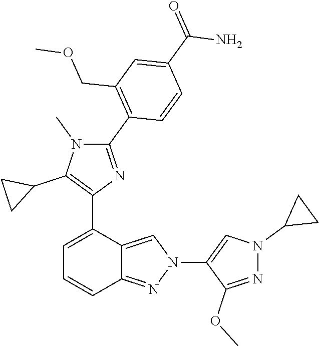 [SMILES: COCc1cc(C(N)=O)ccc1-c1nc(-c2cccc3nn(-c4cn(C5CC5)nc4OC)cc23)c(C2CC2)n1C] 4-{5-cyclopropyl-4-[2-(1-cyclopropyl- 3-methoxy-1H-pyrazol-4-yl)-2H- indazol-4-yl]-1-methyl-1H-imidazol- 2-yl}-3-(methoxymethyl)benzamide | K | 538 | 0.76 |

### Synthesis of Example 7

### Synthesis of 4-(5-cyclopropyl-2-{4-[(S)-ethyl(methyl)phosphoryl]-2-(trifluoromethoxy)phenyl}-1-methyl-1H-imidazol-4-yl)-2-[5-(difluoromethyl)-1-methyl-1H-pyrazol-3-yl]-2H-indazole (Example 7)

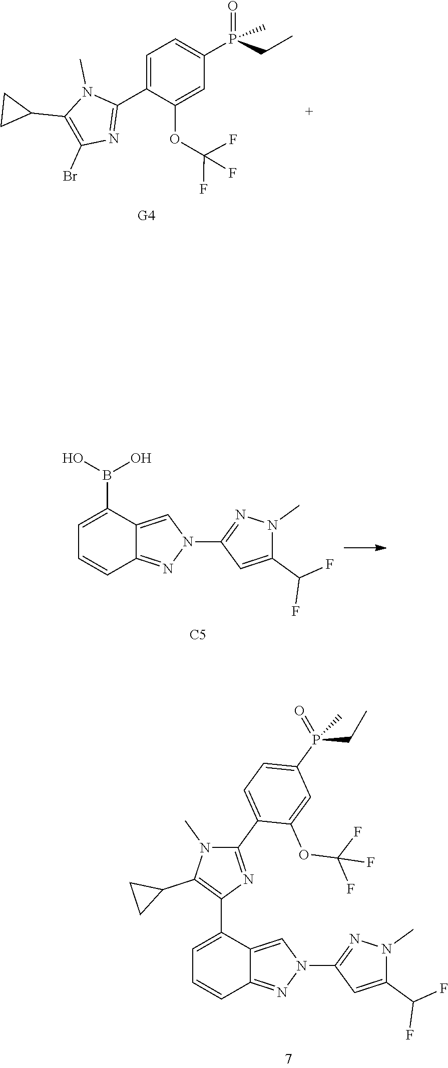 [SMILES: CC[P@](C)(=O)c1ccc(-c2nc(Br)c(C3CC3)n2C)c(OC(F)(F)F)c1 ; Cn1nc(-n2cc3c(B(O)O)cccc3n2)cc1C(F)F ; "C5" ; CC[P@](C)(=O)c1ccc(-c2nc(-c3cccc4nn(-c5cc(C(F)F)n(C)n5)cc34)c(C3CC3)n2C)c(OC(F)(F)F)c1]

4-Bromo-5-cyclopropyl-2-{4-[(S)-ethyl(methyl)phosphoryl]-2-(trifluoromethoxy)phenyl}-1-methyl-1H-imidazole (G4) (29.0 mg, 0.06 mmol) is dissolved in dioxane (2 mL). {2-[5-(Difluoromethyl)-1-methyl-1H-pyrazol-3-yl]-2H-indazol-4-yl}boronic acid (CS) (24.0 mg, 0.08 mmol, 95% purity) and an aqueous solution of Cs₂CO₃ (1 M, 0.20 mL, 0.20 mmol) are added, and the mixture is purged with argon. Pd(dtbpf)Cl₂ (8.00 mg, 0.01 mmol) is added, and the reaction mixture is stirred at 80° C. for 3 h and at rt for 14 h. After the reaction mixture is cooled to RT, it is diluted with DCM (10 mL) and water (5 mL). The organic layer is separated and concentrated. The residue is purified by preparative HPLC (HPLC; ACN/water/TFA) to afford example 7.

Analysis (method C): Rₜ: 0.67 min, [M+H]⁺: 619

### Synthesis of Example 10

### Synthesis of 4-(5-cyclopropyl-2-{4-[(S)-ethyl(methyl)phosphoryl]-2-(trifluoromethoxy)phenyl}-1-methyl-1H-imidazol-4-yl)-2-[1-methyl-5-(trifluoromethyl)-1H-pyrazol-3-yl]-2H-indazole (Example 10)

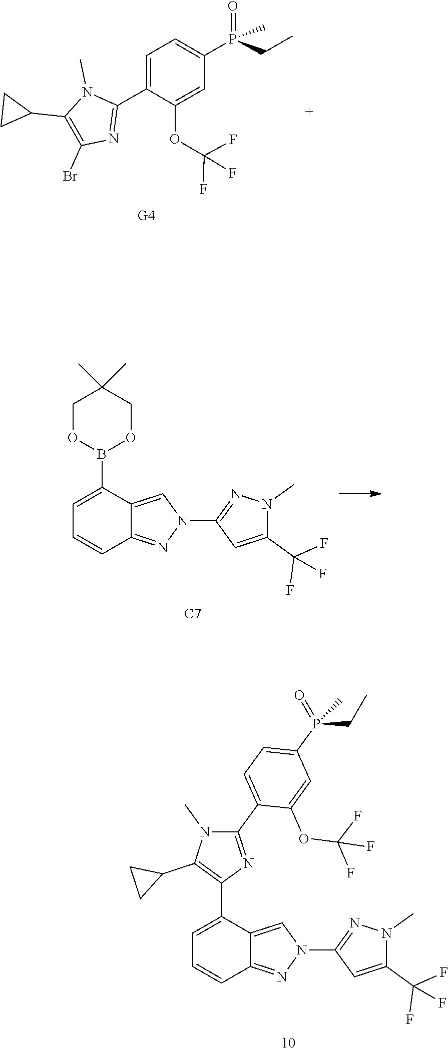 [SMILES: CC[P@](C)(=O)c1ccc(-c2nc(Br)c(C3CC3)n2C)c(OC(F)(F)F)c1 ; Cn1nc(-n2cc3c(B4OCC(C)(C)CO4)cccc3n2)cc1C(F)(F)F ; "C7" ; CC[P@](C)(=O)c1ccc(-c2nc(-c3cccc4nn(-c5cc(C(F)(F)F)n(C)n5)cc34)c(C3CC3)n2C)c(OC(F)(F)F)c1]

4-Bromo-5-cyclopropyl-2-{4-[(S)-ethyl(methyl)phosphoryl]-2-(trifluoromethoxy)phenyl}-1-methyl-1H-imidazole (G4) (33.0 mg, 0.06 mmol, 84% purity) is dissolved in dioxane (3 mL). 4-(5,5-Dimethyl-1,3,2-dioxaborinan-2-yl)-2-[1-methyl-5-(trifluoromethyl)-1H-pyrazol-3-yl]-2H-indazole (C7) (38.3 mg, 0.09 mmol, 91% purity) and an aqueous solution of Cs₂CO₃ (1 M, 0.20 mL, 0.20 mmol) are added, and the mixture is purged with argon. Pd(dtbpf)Cl₂ (4.00 mg, 0.006 mmol) is added, and the reaction mixture is stirred at 80° C. for 3 h and at rt for 16 h. After the reaction mixture is cooled to RT, it is diluted with ACN (5 mL), filtered, and concentrated. The residue is purified by preparative HPLC (HPLC; ACN/water/TFA) to afford example 10.

Analysis (method L): Rₜ: 0.73 min, [M+H]⁺: 637

### Synthesis of Examples 13 and 14

### Step 1: Synthesis of 4-(5-cyclopropyl-2-{4-[ethyl(methyl)phosphoryl]-2-(trifluoromethoxy)phenyl}-1-methyl-1H-imidazol-4-yl)-2-(1-cyclopropyl-3-methoxy-1H-pyrazol-4-yl)-2H-indazole

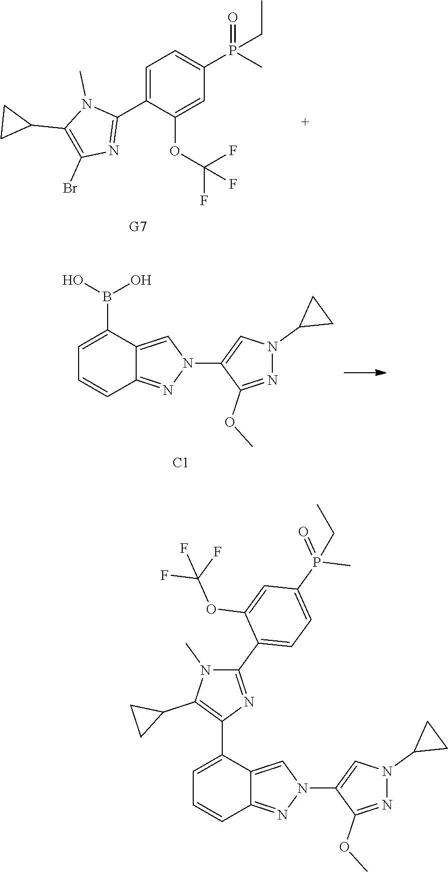 [SMILES: CCP(C)(=O)c1ccc(-c2nc(Br)c(C3CC3)n2C)c(OC(F)(F)F)c1 ; COc1nn(C2CC2)cc1-n1cc2c(B(O)O)cccc2n1 ; "C1" ; CCP(C)(=O)c1ccc(-c2nc(-c3cccc4nn(-c5cn(C6CC6)nc5OC)cc34)c(C3CC3)n2C)c(OC(F)(F)F)c1]

4-Bromo-5-cyclopropyl-2-{4-[ethyl(methyl)phosphoryl]-2-(trifluoromethoxy)phenyl}-1-methyl-1H-imidazole (G7) (50.0 mg, 0.11 mmol) is dissolved in dioxane (2.5 mL). [2-(1-Cyclopropyl-3-methoxy-1H-pyrazol-4-yl)-2H-indazol-4-yl]boronic acid (C1) (50.0 mg, 0.17 mmol) and an aqueous solution of Cs₂CO₃ (2 M, 177 μL, 0.36 mmol) are added, and the mixture is purged with argon. Pd(dtbpf)Cl₂ (7.22 mg, 0.01 mmol) is added, and the reaction mixture is stirred at 80° C. for 4 h. After the reaction mixture is cooled to RT, it is filtered, concentrated and the residue is purified by preparative HPLC (HPLC; ACN/water/TFA) to afford the desired compound.

Analysis (method E): Rₜ: 0.80 min, [M+H]⁺: 625

### Step 2: Synthesis of 4-(5-cyclopropyl-2-{4-[(R)-ethyl(methyl)phosphoryl]-2-(trifluoromethoxy)phenyl}-1-methyl-1H-imidazol-4-yl)-2-(1-cyclopropyl-3-methoxy-1H-pyrazol-4-yl)-2H-indazole (Example 13) and 4-(5-cyclopropyl-2-{4-[(S)-ethyl(methyl)phosphoryl]-2-(trifluoromethoxy)phenyl}-1-methyl-1H-imidazol-4-yl)-2-(1-cyclopropyl-3-methoxy-1H-pyrazol-4-yl)-2H-indazole (Example 14)

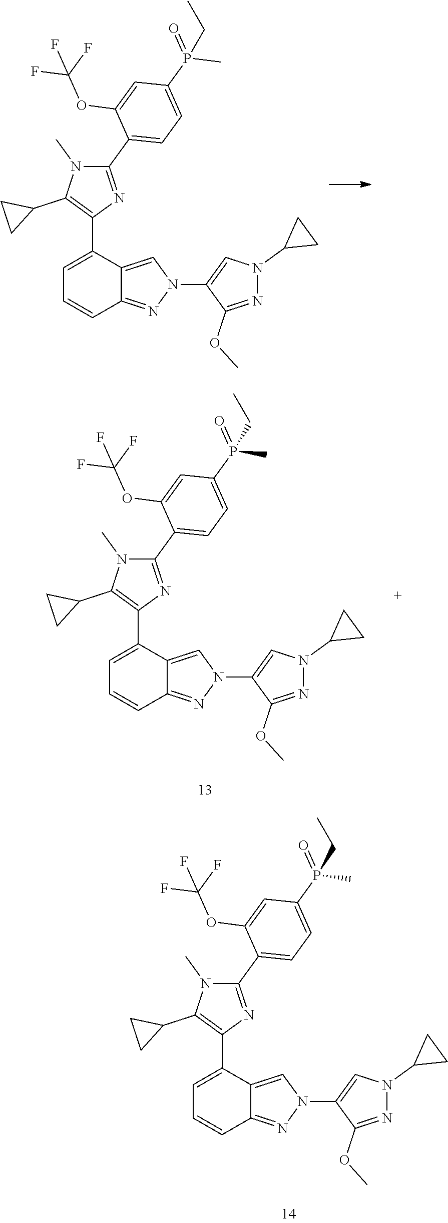

4-(5-Cyclopropyl-2-{4-[ethyl(methyl)phosphoryl]-2-(trifluoromethoxy)phenyl}-1-methyl-1H-imidazol-4-yl)-2-(1-cyclopropyl-3-methoxy-1H-pyrazol-4-yl)-2H-indazole (67.0 mg, 0.11 mmol) is separated by chiral purification method P to afford the compounds 4-(5-cyclopropyl-2-{4-[(R)-ethyl(methyl)phosphoryl]-2-(trifluoromethoxy)phenyl}-1-methyl-1H-imidazol-4-yl)-2-(1-cyclopropyl-3-methoxy-1H-pyrazol-4-yl)-2H-indazole (Example 13) (Analysis (method M): Rₜ: 0.76 min, [M+H]⁺: 625) and 4-(5-cyclopropyl-2-{4-[(S)-ethyl(methyl)phosphoryl]-2-(trifluoromethoxy)phenyl}-1-methyl-1H-imidazol-4-yl)-2-(1-cyclopropyl-3-methoxy-1H-pyrazol-4-yl)-2H-indazole (Example 14) (Analysis (method M): Rₜ: 1.10 min, [M+H]⁺: 625).

### Synthesis of Example 20

### Synthesis of 4-{5-cyclopropyl-2-[4-(dimethylphosphoryl)-2-(trifluoromethoxy)phenyl]-1-methyl-1H-imidazol-4-yl}-2-[1-methyl-5-(trifluoromethyl)-1H-pyrazol-3-yl]-2H-indazole (Example 20)

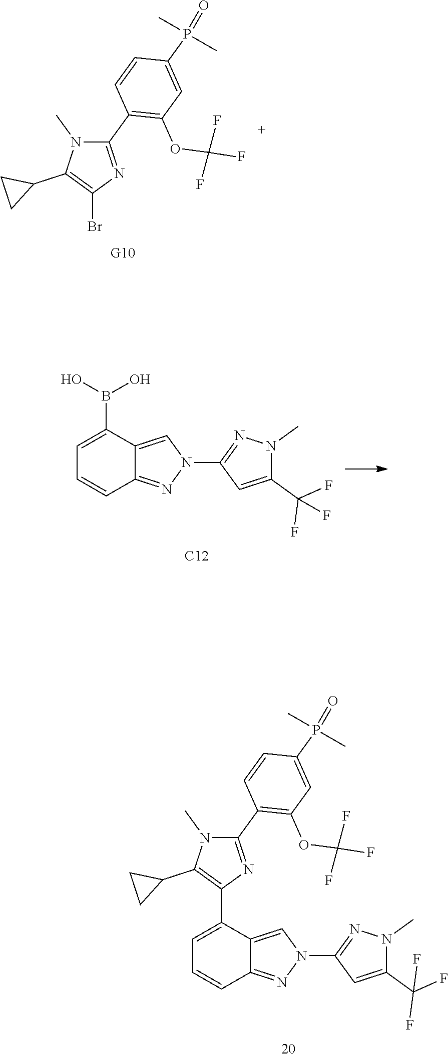 [SMILES: Cn1c(-c2ccc(P(C)(C)=O)cc2OC(F)(F)F)nc(Br)c1C1CC1 ; Cn1nc(-n2cc3c(B(O)O)cccc3n2)cc1C(F)(F)F ; "C12" ; Cn1nc(-n2cc3c(-c4nc(-c5ccc(P(C)(C)=O)cc5OC(F)(F)F)n(C)c4C4CC4)cccc3n2)cc1C(F)(F)F]

4-Bromo-5-cyclopropyl-2-[4-(dimethylphosphoryl)-2-(trifluoromethoxy)phenyl]-1-methyl-1H-imidazole (G10) (65.0 mg, 0.118 mmol), {2-[1-methyl-5-(trifluoromethyl)-1H-pyrazol-3-yl]-2H-indazol-4-yl}boronic acid (C12) (55.0 mg, 0.177 mmol), K₃PO₄ (2 M, 176 μL, 0.35 mmol) and XPhos Pd G3 (20.0 mg, 0.0232 mmol) are dissolved in dioxane (2 mL). The mixture is purged with argon and is stirred at 80° C. for 14 h. After the reaction mixture is cooled to RT, it is filtered, concentrated and the residue is purified by preparative HPLC (HPLC; ACN/water/TFA) to afford example 20.

Analysis (method C): Rₜ: 0.70 min, [M+H]⁺: 623

### Synthesis of Example 21

### Synthesis of 4-{5-cyclopropyl-2-[4-(dimethylphosphoryl)-2-(trifluoromethoxy)phenyl]-1-methylimidazol-4-yl}-2-[5-(trifluoromethyl)-1H-pyrazol-3-yl]indazole (Example 21)

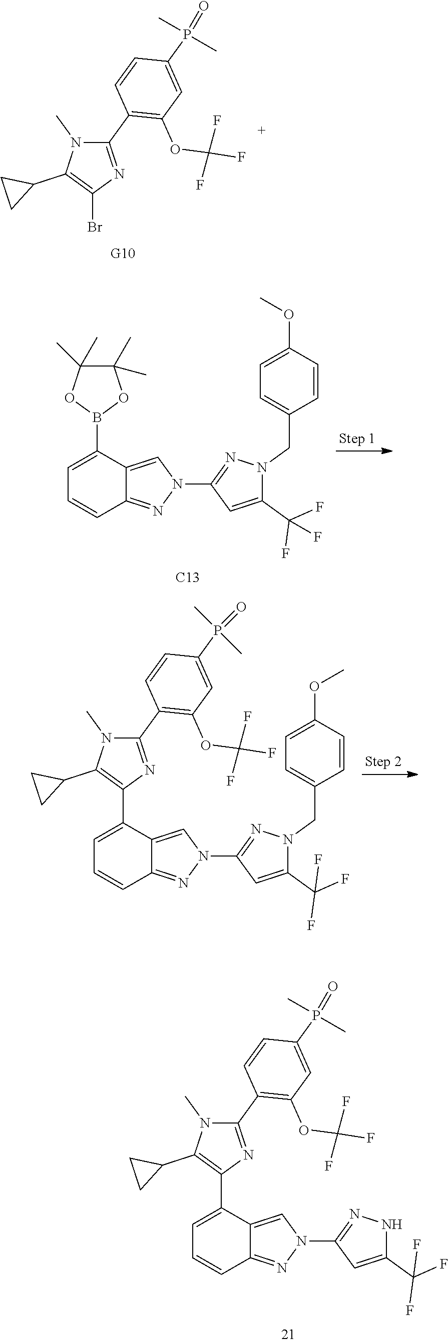 [SMILES: Cn1c(-c2ccc(P(C)(C)=O)cc2OC(F)(F)F)nc(Br)c1C1CC1 ; COc1ccc(Cn2nc(-n3cc4c(B5OC(C)(C)C(C)(C)O5)cccc4n3)cc2C(F)(F)F)cc1 ; "C13" ; COc1ccc(Cn2nc(-n3cc4c(-c5nc(-c6ccc(P(C)(C)=O)cc6OC(F)(F)F)n(C)c5C5CC5)cccc4n3)cc2C(F)(F)F)cc1 ; Cn1c(-c2ccc(P(C)(C)=O)cc2OC(F)(F)F)nc(-c2cccc3nn(-c4cc(C(F)(F)F)[nH]n4)cc23)c1C1CC1]

### Step 1

4-Bromo-5-cyclopropyl-2-[4-(dimethylphosphoryl)-2-(trifluoromethoxy)phenyl]-1-methyl-1H-imidazole (G10) (100 mg, 0.226 mmol), C13 (mixture of regioisomers, 135 mg, 0.272 mmol), K₃PO₄ (2 M, 340 μL, 0.68 mmol) and XPhos Pd G3 (14 mg, 0.016 mmol) are dissolved in 2-methyltetrahydrofuran (2 mL). The mixture is purged with argon and is stirred at 80° C. for 3 h. After the reaction mixture is cooled to RT, it is filtered, concentrated and the residue is purified by preparative HPLC to afford an intermediate used for step 2.

Analysis (method A): Rₜ: 0.63 min, [M+H]⁺: 729

### Step 2

4-{5-Cyclopropyl-2-[4-(dimethylphosphoryl)-2-(trifluoromethoxy)phenyl]-1-methyl-1H-imidazol-4-yl}-2-{1-[(4-methoxyphenyl)methyl]-5-(trifluoromethyl)-1H-pyrazol-3-yl}-2H-indazole (60 mg, 0.082 mmol) is dissolved in TFA (1 mL) and anisol (9 μL) and stirred at 120° C. for 20 min under microwave. The mixture is diluted with acetonitrile and concentrated. The residue is dissolved in acetonitrile and purified via reversed phase chromatography to afford example 21.

Analysis (method A): Rₜ: 0.51 min, [M+H]⁺: 609

The example 22

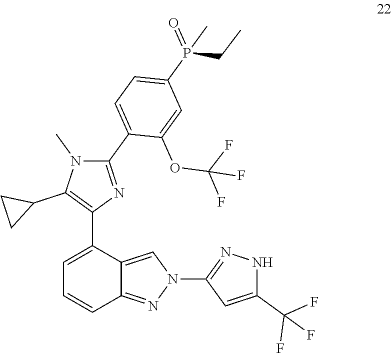 [SMILES: CC[P@](C)(=O)c1ccc(-c2nc(-c3cccc4nn(-c5cc(C(F)(F)F)[nH]n5)cc34)c(C3CC3)n2C)c(OC(F)(F)F)c1]

is obtained by following a reaction sequence analogous to that described for example 21, using the intermediate G4 (see above) instead of G10.

By analogous methods to the methods disclosed herein above the other compounds shown in Table 2A can be synthesized using the corresponding intermediates.

### Analytical HPLC Methods

Method A (X012_S01)

| | Vol % water | | |
|---|---|---|---|
| time (min) | (incl. 0.1% TFA) | Vol % ACN | Flow [mL/min] |
| 0.00 | 99 | 1 | 1.6 |
| 0.02 | 99 | 1 | 1.6 |
| 1.00 | 0 | 100 | 1.6 |
| 1.10 | 0 | 100 | 1.6 |

Device description: Waters Acquity; Analytical column: Xbridge (Waters) BEH C18_2.1 × 30 mm_1.7 μm; column temperature: 60° C.

Method B (Z011_S03)

| | Vol % water | | |
|---|---|---|---|
| time (min) | (incl. 0.1% NH₃) | Vol % ACN | Flow [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Xbridge (Waters) C18_3.0 × 30 mm_2.5 μm; column temperature: 60° C.

Method C (X018_S03)

| | Vol % water | | |
|---|---|---|---|
| time (min) | (incl. 0.1% TFA) | Vol % ACN | Flow [mL/min] |
| 0.00 | 95 | 5 | 1.5 |
| 1.30 | 0 | 100 | 1.5 |
| 1.50 | 0 | 100 | 1.5 |

Device description: Waters Acquity; Analytical column: Sunfire (Waters) C18_3.0 × 30 mm_2.5 μm; column temperature: 60° C.

Method D (X011_S05)

| | Vol % water | | Flow |
|---|---|---|---|
| time (min) | (incl. 0.1% NH₃) | Vol % ACN | [mL/min] |
| 0.00 | 95 | 5 | 1.3 |
| 0.02 | 95 | 5 | 1.3 |
| 1.00 | 0 | 100 | 1.3 |
| 1.30 | 0 | 100 | 1.3 |

Device description: Waters Acquity;
Analytical column: Xbridge (Waters) BEH C18_2.1 × 30 mm_2.5 μm;
column temperature: 60° C.

Method E (Z018_S04)

| | Vol % water | | Flow |
|---|---|---|---|
| time (min) | (incl. 0.1% TFA) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200;
Analytical column: Sunfire (Waters) C18_3.0 × 30 mm_2.5 μm;
column temperature: 60° C.

Method F (SLV-Method-111)

| | Vol % water | | Flow |
|---|---|---|---|
| time (min) | (incl. 0.05% NH₃) | Vol % ACN | [mL/min] |
| 0.00 | 80 | 20 | 1.0 |
| 3.35 | 20 | 80 | 1.0 |
| 3.75 | 20 | 80 | 1.0 |
| 3.90 | 5 | 95 | 1.0 |
| 4.75 | 5 | 95 | 1.0 |
| 5.00 | 80 | 20 | 1.0 |
| 6.00 | 80 | 20 | 1.0 |

Device description: Dionex UHPLC Ultimate 3000;
Analytical column: Kinetex XB-C18_4.6 × 50 mm_2.6 μm;
column temperature: 25° C.

Method G (I_IH_15_IPA_NH3_003)

| | | Vol % IPA | Flow |
|---|---|---|---|
| time (min) | Vol % scCO₂ | (incl. 20 mM NH₃) | [mL/min] |
| 0.00 | 85 | 15 | 4.0 |
| 10.00 | 85 | 15 | 4.0 |

Device description: Agilent 1260 Infinity II SFC;
Analytical column: Chiralpak ® (Daicel) IH_4.6 × 250 mm_5 μm;
column temperature: 40° C.;
back pressure: 2175 psi

Method H (Z020_S01)

| | Vol % water | | Flow |
|---|---|---|---|
| time (min) | (incl. 0.1% FA) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200;
Analytical column: Sunfire (Waters) C18_3.0 × 30 mm_2.5 μm;
column temperature: 60° C.

Method I (008_CA02)

| | Vol % water | | Flow |
|---|---|---|---|
| time (min) | (incl. 0.1% NH₃) | Vol % ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity;
Analytical column: Xbridge (Waters) C18_3.0 × 30 mm_2.5 μm;
column temperature: 60° C.

Method J (008_CA11)

| | Vol % water | | Flow |
|---|---|---|---|
| time (min) | (incl. 0.1% NH₃) | Vol % ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity;
Analytical column: Xbridge (Waters) C18_3.0 × 30 mm_2.5 μm;
column temperature: 60° C.

Method K (008_CA10)

| | Vol % water | | Flow |
|---|---|---|---|
| time (min) | (incl. 0.1% NH₃) | Vol % ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity;
Analytical column: Xbridge (Waters) C18_3.0 × 30 mm_2.5 μm;
column temperature: 60° C.

Method L (007_CA11)

| | Vol % water | Vol % ACN | Flow |
|---|---|---|---|
| time (min) | (incl. 0.1% TFA) | (incl. 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity;
Analytical column: Sunfire (Waters) C18_3.0 × 30 mm_2.5 μm;
column temperature: 60° C.

Method M (I_AC_20_MEOH_NH3_002)

| | | Vol % MeOH | Flow |
|---|---|---|---|
| time (min) | Vol % scCO₂ | (incl. 20 mM NH₃) | [mL/min] |
| 0.00 | 80 | 20 | 2.0 |
| 4.00 | 80 | 20 | 2.0 |

Device description: Agilent 1260 Infinity II SFC;
Analytical column: Chiralpak ® (Daicel) IH_4.6 × 250 mm_5 μm;
column temperature: 40° C.;
back pressure: 2175 psi

Method N (Z017_S04)

| | Vol % water | | Flow |
|---|---|---|---|
| time (min) | (incl. 0.1% TFA) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200;
Analytical column: StableBond (Zorbax) C18_3.0 × 30 mm_1.8 μm;
column temperature: 60° C.

Method O

| Column | Chiralpak ® IH 20 × 250 mm_5 μm | |
|---|---|---|
| Solvents: | | |
| scCO2 | 95% | |
| MeOH + 20 mM NH3 | 5% | |
| Backpressure Regulator | 150 | bar |
| Temperature | 40° | C. |
| Flowrate | 60 | ml/min |
| Sample concentration | 30 | mg/ml |
| Sample solvent | MeOH | |
| Injection volume | 100 | μl |
| Detector wavelength | 220 | nm |

| | |
|---|---|
| Device | Sepiatec I Prep SFC 100 |

Method P

| Column | CHIRAL ART ® Amylose-C_neo_10 × | |
|---|---|---|
| | 250 mm_5 μm | |
| Solvents: | | |
| scCO2 | 80% | |
| MeOH + 20 mM NH3 | 20% | |
| Backpressure Regulator | 150 | bar |
| Temperature | 40° | C. |
| Flowrate | 15 | ml/min |
| Sample concentration | 13 | mg/ml |
| Sample solvent | MeOH | |
| Injection volume | 100 | μl |
| Detector wavelength | 220 | nm |

| | |
|---|---|
| Device | Sepiatec PrepSFC50 |

### List of Abbreviations:

Ac acetyl
ACN acetonitrile
AIBN 2,2′-azobis(isobutyronitrile)
Boc tert-butyloxycarbonyl
Cbz benzyloxycarbonyl
CycH cyclohexane
d day(s)
DAST diethylamino sulfur trifluoride
DCE 1,2-dichloroethane
DCM dichloromethane
DEAD diethyl azodicarboxylate
DIAD diisopropyl azodicarboxylate
DIPEA N,N-diisopropylethylamine
DMF N,N-dimethylformamide
DMP Dess-Martin Periodinane
DMSO dimethyl sulfoxide
EtOAc ethyl acetate
EtOH ethanol
h hour(s)
HATU O-(7-azabenzotriazol-1-yl)-N,N,N′,N′-tetramethyluronium-hexafluorophosphate
HPLC high performance liquid chromatography
HPLC-MS coupled high performance liquid chromatography-mass spectrometry
IPA isopropyl alcohol
LC liquid chromatography
LC-MS coupled liquid chromatography—mass spectrometry
LiHMDS Lithium-bis(trimethylsilyl)amide
M molar (mol/L)
MeI methyl iodide
MeTHF 2-methyltetrahydrofuran
MeOH methanol
min minute(s)
MS mass spectrometry
MTBE methyl-tertbutyl-ether
n-BuLi n-Buthyllithium
NBS N-Bromosuccinimide
NIS N-Iodosuccinimide
NMP N-methyl-2-pyrrolidone
NMR nuclear magnetic resonance
PEPPSI(TM)-IPR (1,3-Bis(2,6-diisopropylphenyl)imidazolidene) (3-chloropyridyl) palladium(II) dichloride
PdCl2(dtbpf) 1,1′-Bis-(di-tert-butylphosphino-)ferrocene-palladiumdichloride
Pd(dppf)Cl₂ 1,1′-bis(diphenylphosphino)ferrocenedichloropalladium(II)
Pd(PPh₃)₄ palladium (0) tetrakis(triphenylphosphine)
XPhos Pd G3 2-Dicyclohexylphosphino-2′,4′,6′-triisopropyl-1,1′-biphenyl)[2-(2′-amino-1,1′-biphenyl)]palladium(II) methanesulfonat
pet. petroleum
R_{f} retention factor
RP reverse phase
rt room temperature
t_{R} retention time (in HPLC/LC)
SFC supercritical fluid chromatography
TBAF tetrabutylammonium fluoride
TBTU O-(benzotriazol-1-yl)-N,N,N′,N′-tetramethyluronium tetrafluoroborate
TEA triethylamine
TFA trifluoroacetic acid
THF tetrahydrofuran
THP tetrahydro-2h-pyran
TLC thin-layer chromatography
TMAD N,N,N′N′-Tetramethylazodicarboxamide
UV ultraviolet
V volume

### Biological Assays and Data

The activity of the compounds of the invention may be demonstrated using the following in vitro STING biochemical and cell assays.

### Human STING HTRF Binding Assay

Binders to human STING WT (R232) were identified using a competitive HTRF assay format (Cisbio 64BDSTGPEG), which uses d2-labeled STING ligand, a 6His tagged human STING protein, and an anti 6His Cryptate-labeled antibody. Compounds compete with the STING ligand-d2 and thereby prevents FRET from occurring, which can be measured by an EnVision™ reader (PerkinElmer).

Assay method: Compounds were delivered as 10 mM DMSO solution, serially diluted by an Agilent Bravo Workstation and transferred to the 384 well assay plate (Perkin Elmer #6005359) using a Cybiwell dispenser. Typically, 8 concentrations were used with the highest concentration at 10 μM or 1 μM in the final assay volume followed by ^{˜}1:5 dilution steps. DMSO concentration was set to 1% in the final assay volume. The 384 well assay plate contained 20 test compounds and DMSO in column 23 and 24. A cGAMP standard dilution row was prepared according to the manufacturer and transferred to each assay plate. After transfer of compound solution or dilution buffer for negative (high) and positive (low) controls, 5 μl of the human STING protein (cyclic binding domain (residues 138-379) of the WT R232 human version, fused to a 6 His tag at the Nter part; 1:50 dilution in detection buffer) were dispensed to all wells except of the positive control, which received detection buffer only. Plates were the centrifuged for 20 sec at 1000 rpm. After that, 10 μl of Anti-6His-Cryptate antibody/Sting ligand-d2 mix was added to all wells using a Multidrop combi dispenser, followed by another 20 sec/1000 rpm centrifugation step. After an incubation of the plates for 180 min at room temperature, excitation at 665/620 nM (HTRF ratio) was measured using an Envison Reader (PerkinElmer)

Data evaluation and calculation: For data evaluation and calculation, HTRF ratios were calibrated using the cGAMP standard curve. After that, the measurement of the low control was set as 0% control and the measurement of the high control was set as 100% control. The IC50 values were calculated using the standard 4 parameter logistic regression formula. Calculation: [y=(a−d)/(1+(x/c)\hat{ }b)+d], a=low value, d=high value; x=conc M; c=IC50 M; b=slope;

The results of this assay are shown in the characterising data table below.

### Determination of the Increase of Stability of STING Protein Against Thermal Denaturation, Differential Scanning Fluorimetry (DSF)

The binding affinity of the compounds of the invention may be demonstrated using a thermal shift assay that measures the stability of a suitable protein material of human STING against thermal denaturation in the presence of compounds. In this assay, the unfolding temperature of a protein is monitored in the presence of a fluorescent dye which exhibits affinity for the hydrophobic amino acids of the protein that are buried in its folded state and are gradually exposed during unfolding. Dye fluorescence is quenched in aqueous environment and increases upon association of the dye with the hydrophobic parts of the unfolding protein. A plot of the fluorescence intensity as a function of temperature typically displays a sigmoidal curve that is interpreted by a two-state model of protein unfolding (Differential Scanning Fluorimetry). The inflection point of the curve represents the “melting” temperature of the protein (Tm) which is calculated numerically using the Boltzmann equation.

Method: The thermal stability of the STING protein was measured using a specific expression construct of the cGAMP binding domain of wild-type (GRR) human STING comprising residues 155-341 and a N-terminal 8x His-tag in assay buffer containing 20 mM Tris, 150 mM NaCl at pH7.5.

The assay uses Hard-Shell®PCR Plates 384-Well CLR/WHT (Catalog #HSP3805, BIO-RAD), Microseal®‘B’ Adhesive Seals for PCR Plates (Catalog #MSB-1001, BIO-RAD) and was run on a CFX384 Real-Time System (Bio-Rad).

A DMSO stock solution of SYPRO orange (SIGMA S5692-500UL) was prepared.

Compound stock solutions (10 mM in DMSO) were diluted 1:2 in DMSO to an intermediate compound concentration of 5 mM and then further diluted 1:40 in assay buffer resulting in a compound concentration of 125 μM and 2.5% DMSO.

Fluorescent dye stock solution (5000×SYPRO Orange) was then mixed with target protein and buffer to a concentration of 15 uM Protein and 25×SYPRO Orange. 2 ul of this protein-dye-mixture was added to 8 ul compound solution. Final volume was 10 uL. 3-6 well positions were used as negative control (protein with 2% DMSO). The plates were prepared for duplicate measurement and centrifuged for 2 min at 1000 g. In the measurement, 160 cycles of 0.5° C. were used (temperature ramp 15 s/cycle, 15° C. to 95° C.).

Final Assay concentrations for compound characterization were as follows:

100 uM compound, 3 uM target protein, 5×SYPRO Orange, 2% DMSO in 10 ul. All dispensing steps were performed using a HamiltonStar pipetting robot (Hamilton).

Dissociation curves were processed in Bio-Rad CFX Manager. Peak type was set to “negative”.

Compound codes for screen were assigned in the plate layout.

Two replicates of TM measurements were averaged, and the standard deviation was calculated. In cases of SD>1.5° C. the measurement was repeated.

The melting point (Tm) obtained for STING protein alone was subtracted from T obtained for protein incubated with ligand to generate ΔTm values.

Protein production and purification: The protein used for the biophysical experiments was a recombinant human STING protein comprising its cytosolic ectodomain. A codon optimized DNA sequence (for expression in Escherichia coli) encoding amino acid residues 155 to 341 (Swiss Prot Q86WV6) of human STING (WT) was synthesized by GeneArt (Regensburg, Germany) and inserted into a pET17b E. coli expression vector. The protein construct encodes an N-terminal 8× His-tag followed by tobacco etch virus protease (TEV) cleavage site and the above STING gene sequence. The resulting protein sequence for the used STING variant is listed below:

His-TEV-hSTING (WT)
(SEQ ID NO: 1)
MHHHHHHHHENLYFQSGVAHGLAWSYYIGYLRLILPELQARIRTYNQH
YNNLLRGAVSQRLYILLPLDCGVPDNLSMADPNIRFLDKLPQQTGDRA
GIKDRVYSNSIYELLENGQRAGTCVLEYATPLQTLFAMSQYSQAGFSR
EDRLEQAKLFCRTLEDILADAPESQNNCRLIAYQEPADDSSFSLSQEV
LRHLRQEEKEEV

For expression of recombinant human STING above construct was transformed into E. coli BL21 DE3 strain and grown in shake flasks in LB-medium at 37° C. Expression was induced by addition of isopropyl β-D-1-thiogalactopyranoside to a final concentration of 1 mM and cultures shaken overnight. Cell pellets were centrifuged and stored at −70° C. until further use. Protein was purified by cell thawing in lysis buffer (20 mM TRIS-HCl, pH 8, 300 mM NaCl, 2 mM mercaptoethanol, 20 mM imidazole, Complete Protease Inhibitor (Roche) and DNase (Roche)), followed by metal affinity purification using Ni-NTA resins and elution buffer consisting of 20 mM TRIS-HCl, pH 8, 300 mM NaCl, 2 mM mercaptoethanol, 300 mM imidazole and size exclusion chromatography in running buffer (20 mM TRIS-HCl, pH 8, 100 mM NaCl, 2 mM DTT). The peak fraction was collected and concentrated to 2.5 mg/mL.

The results of this assay are shown in the characterising data table below.

### Human Whole Blood Assay (HWBA)

For the detection of STING inhibition in physiological environment human whole blood was stimulated by the cyclic dinucleotide cGAMP. Pathway activity was monitored by measuring the IFNα2α production.

Assay method: Compounds were delivered as 10 mM DMSO solution and serial diluted and transferred to the 96-well Cell culture Plate (Corning #3595), prefilled with 20 μl OptiMEM (Gibco #11058-021) in each well, using an Echo acoustic dispenser. Typically, 8 concentrations were used with the highest concentration at 10 μM in the final assay volume followed by ^{˜}1:5 dilution steps. DMSO concentration was set to 0.1% in the final assay volume. The 96 well assay plate contained 9 test compounds, a reference compound and DMSO in control wells.

Collection of human whole blood from 3 or more healthy donors (male or female, no medication for 7 days, exception contraceptive and thyroxine) as Na-citrate blood (e.g. 3.8% in Monovettes from Sarstedt) is conducted in parallel. Whole blood was kept at room temperature for a maximum of 3 hours after collection until use in the assay.

160 μl of the whole blood samples were transferred to each well of the 96-well assay plates filled with compound/OptiMEM. All assay plates are prepared as duplicates with blood from different donors. Blood plates were kept at room temperature for 60 minutes and continuous shaking with 450 rpm, covered with the lid, but not sealed.

A 10× cGAMP assay solution was diluted from a 2 mM stock solution in 1×HBSS immediately before use at room temperature. 20 μl of the 10× cGAMP/HBSS were added to all compound and all high control wells, whereas HBSS only was added to all low control wells.

After covering assay plates with aera seals and the lid, blood plates were kept at room temperature for 30 minutes and continuous shaking with 450 rpm, followed by an overnight incubation of 22 h at 37° C. in the incubator, without shaking.

For the detection of IFNα-2α in human plasma, the biotinylated capture antibody (Antibody set IFNA2, Meso Scale Diagnostics #B21VH-3, including coating and capture antibody) was diluted 1:17.5 in Diluent 100 (Meso Scale Diagnostics #R50AA-4, according to the manufacturer. U-Plex MSD GOLD 96-well Small Spot Streptavidin SECTOR Plates (Meso Scale Diagnostics #L45SA-5) were coated with 25 μl diluted capture antibody. Coated plates were incubated for 60 min at room temperature under continuous shaking at 700 rpm. MSD IFNα-2α plates were washed three times with 150 μl wash buffer (1×HBSS, 0.05% Tween).

After blocking the plates with 100 μl block solution/well (1×HBSS with 0.2% Tween, 2% BSA) for 60 min at room temperature and continuous shaking at 700 rpm, plates were emptied as dry as possible by dumping just before continuing with the human plasma. Whole Blood assay plates were centrifuged at 1600 rpm for 10 minutes. 25 μl of supernatant was transferred with a pipetting robotics from each whole blood plate to the corresponding IFNα-2α plate. Plates were sealed with microplate seals and kept at room temperature again under continuous shaking at 700 rpm for two hours. Next MSD IFNα-2α plates were washed three times with 150 μl wash buffer (1×HBSS, 0.05% Tween), before adding 25 μl MSD SULFO-TAG IFNα-2α Antibody solution (1:100 diluted in Diluent 3 (Meso Scale Diagnostics #R50AP-2) to each well of the plates. Afterwards plates were sealed with microplate seals and kept at room temperature again under continuous shaking at 700 rpm for two hours. Finally, MSD IFNα-2α plates were washed three times with 150 μl wash buffer (1×HBSS, 0.05% Tween). 150 μl 2× Read buffer was added to each well and plates were immediately measured with the MSD Sector S600 Reader using the vendor barcode.

Data evaluation and calculation: For data evaluation and calculation, % control calculation of each well was based on the mean of high (cGAMP stimulated control) and mean of low (unstimulated control) controls by using the following formula:

$$[ counts ( sample ) - counts ( low ) ) / ( counts ( high ) - counts ( low ) ) ] * 100$$

The IC50 values were calculated using the standard 4 parameter logistic regression formula.

Calculation: [y=(a−d)/(1+(x/c)\hat{ }b)+d], a=low value, d=high value; x=conc M; c=IC50 M; b=slope;

The results of this assay are shown in the characterising data table below.

### Human STING Reporter Gene Assay

A THP1-BlueISG reporter cell line expressing wildtype STING and IRF dependent alkaline phosphatase reporter was used for the potency measurement of activators of human wildtype STING.

Assay Method: Compounds were delivered 10 mM DMSO solution and serially diluted in assay medium (RPMI 1640 (Life Technologies #A10491-01), 10% FCS (Life Technologies #10500-064), 1× Pen/Strep solution (Life Technologies #15140-122). Typically, 8 concentrations were used with the highest concentration at 10 or 100 μM in the final assay volume followed by ^{˜}1:5 dilution steps. DMSO concentration was set to 1% in the final assay volume. The 384 well assay plate contained 21 test compounds (column 1-21), a reference compound (column 22) and DMSO in column 23 and 24;

Cells, cultivated according to manufacturer's conditions (culture medium: RPMI 1640 (Life Technologies #A10491-01), 10% FCS (Life Technologies #10500-064), 1× Pen/Strep solution (Life Technologies #15140-122), 100 μg/mL Normocin (Life Technologies #ant-nr-1), 100 μg/mL Zeocin (Life Technologies #R25001) were harvested, resuspended, and diluted in fresh assay medium. The cells were then seeded in 15 μl assay media to the assay plates (10000 cells/well), followed by addition of 5 μl prediluted compound solution to wells of the assay plates. Afterwards 5 ul per well of assay medium was added to the wells containing compounds, followed by a 30 min incubation at RT and a 24 h incubation at 37° C. Then 5 ul per well of assay medium with DMSO (1% f.c.) was added to the wells for the controls, plus 5 μl of assay medium alone for negative controls (low values) or 5 μl of prediluted 2′3′-cGAMP (20 μM f.c.; BIOLOG Life Science Institute #C 161 or Invivogen #tlrl-nacga23) for positive controls (high values).

Finally, 75 μl of Quanti Blue reagent was added to the plates using a MultiDrop Combi, followed by 30 min incubation at 37° C. The absorbance was measured on the EnVision™ reader (PerkinElmer).

Data evaluation and calculation: For data evaluation and calculation, the measurement of the low control was set as 100% control and the measurement of the high control was set as 200% control. The EC50 values were calculated using the standard 4 parameter logistic regression formula. Calculation: [y=(a−d)/(1+(x/c)\hat{ }B)+d], a=low value, d=high value; x=conc M; c=IC50 M; b=slope;

The results of this assay are negative for agonism, wherein the threshold was set larger than 30 μM.

Characterising Data Table

| | STING HTRF | DSF | HWBA |
|---|---|---|---|
| Example | IC50 (nM) | ΔTm (K) | IC50 (μM) |
| 1 | | 28 | 0.082 |
| 2 | 25.36 | 30 | 0.21 |
| 3 | 122.00 | 26 | 2.6 |
| 4 | 40.89 | 30 | 0.095 |
| 5 | 30.27 | 28 | 0.81 |
| 6 | 15.99 | 28 | 0.058 |
| 7 | 19.66 | 30 | 0.057 |
| 8 | 85.33 | 32 | 0.08 |
| 9 | 17.57 | 22 | 1.5 |
| 10 | 31.89 | 30 | 0.078 |
| 11 | 42.33 | 26 | 0.88 |
| 12 | 9.60 | 25 | 0.84 |
| 13 | 18.89 | 27 | 0.038 |
| 14 | 6.49 | 30 | 0.011 |
| 15 | 32.88 | 25 | 0.11 |
| 16 | 13.85 | 32 | 0.035 |
| 17 | 48.14 | 29 | 0.089 |
| 18 | 26.53 | 34 | 0.087 |
| 19 | 17.98 | 28 | 0.14 |
| 20 | 49.10 | 30 | 0.18 |
| 21 | | 29 | 0.13 |

As shown by the characterizing data, the inventive compounds can inhibit STING and by doing so are advantageous in the prevention, delaying and/or treatment of diseases or conditions which can be influenced by STING inhibition, for example but not limited to those disclosed herein above. In a preferred embodiment, the inventive compounds have in a competitive HTRF assay format (Cisbio 64BDSTGPEG) an IC50 value of at least and including 0.3 nM and not more than 250 nM, preferably not more than 150 nM, more preferably not more than 125 nM and even more preferably not more than 70 nM. In another preferred embodiment, said IC 50 value is at least and including 0.8 nM or at least and including 2 nM. In another preferred embodiment said IC50 value is not more than 45 nM, more preferably not more than 40 nM.

### Further Characterization

Efflux Ratio from MDCK-PGP

The efflux ratio from MDCK-PGP cells is measured using standard methods according to the international patent application published as WO24089006 or as in the publication by Dong et al. Pharm Res (2020) 37: 194, https://doi.org/10.1007/s11095-020-02895-9.

In one embodiment the efflux ratio in MDCK-PgP cell is equal to or below 30, preferably equal to or below 25 more preferably equal to or below 15. In a more preferred embodiment, the efflux ratio is less than 15 but higher than 0.5. Exemplary values are ratios of 22 or 13.

Efflux Ratio from CACO2 Cells

The efflux ratio from CACO2 cells is determined using standard methods for example as disclosed in the international patent applications published as WO15048318, WO22254371 and WO24110851, or as in the publication by Dong et al. Pharm Res (2020) 37: 194, https://doi.org/10.1007/s11095-020-02895-9.

In one embodiment the CACO2 cell efflux ratio of the inventive compounds is equal to or below 12, 10, 8, 7, 6, 5, 4.5, 4, 3.5, 3, 2.5, 2, 1.5, 1.3. In one embodiment the efflux ratio is above 0.7. An exemplary efflux value is around 1.

### Inhibition of Cytochrome P450 Enzymes CYP2D6 and CYP3A4

Standard assays for testing the inhibition of cytochrome P450 enzymes using typical substrates are known in the art. For example, the susbtrate dextromethorphan is known to be primarily metabolized by CYP2D6 (Schadel M, Wu D, Otton S V, Kalow W, Sellers E M. Pharmacokinetics of dextromethorphan and metabolites in humans: influence of the CYP2D6 phenotype and quinidine inhibition. J Clin Psychopharmacol. 1995 August; 15(4):263-9. doi: 10.1097/00004714-199508000-00005. PMID: 7593709.) and inhibiting effects of new compounds on the metabolization of dextromethorphan in human liver microsomes by drug-drug-interaction are commonly used (see for example experimental sections of the patent applications published as WO15073310& WO14197345 and the U.S. Pat. No. 8,138,188 BB).

For testing the possible inhibition of the compounds of the invention, demethylation of Dextromethorphan (5 μM) by the test compound at five different concentrations or no compound (high control) is assayed at 37° C. with human liver microsomes and measured with LC-MS/MS. The IC50 values of the compounds are determined. The IC50 of a positive control inhibitor (quinidine) is also determined as a control.

Similar assay systems using human liver microsomes for the possible inhibition of other cytochrome P450 enzymes for example CYP3A4 are known (see for example experimental sections of the patent applications published as WO15073310& WO14197345 and the U.S. Pat. No. 8,138,188 BB). For testing the possible inhibition of the compounds of the invention, hydroxylation of Midazolam (5 μM) by the test compound at five different concentrations or no compound (high control) is assayed at 37° C. with human liver microsomes and measured with LC-MS/MS. The IC50 values of the compounds are determined. The IC50 of a positive control inhibitor (ketoconazole) is also determined as a control.

CYP3A4 and/or CYP2D6 inhibition is observed for the inventive compounds with IC50 values of equal to or greater 1 μmol, preferably equal to or greater 10 μmol and more preferred equal to or greater 20 μmol and even more preferred equal to or greater 25 μmol and most preferred over 30 μmol.

Measuring Clearance from Human Hepatocytes

The metabolic degradation of a test compound is assayed in a human hepatocyte suspension using known methods as in the patent application. US2024327429.

In one embodiment the hepatocyte clearance is lower than 25% Qₕ [%], preferably equal to or lower than 20%, 15%, 10%, 8%, 7%, 6%, 5%, 4%, 3% or 2%. Exemplary values of inventive compounds are 6.5% and about 2%.

### Plasma Protein Binding

Plasma protein binding of a test compound is assessed with known methods, for example as known from the international patent application WO17004537 or the more recent WO25036713. The equilibrium dialysis technique is used to determine the approximate in vitro fractional binding of test compounds to plasma proteins applying Dianorm Teflon dialysis cells (micro 0.2). Each dialysis cell consists of a donor and an acceptor chamber, separated by an ultrathin semipermeable membrane with a 5 kDa molecular weight cutoff. Stock solutions for each test compound are prepared in DMSO at 1 mM and serially diluted to obtain a final test concentration of 1 μM. The subsequent dialysis solutions are prepared in plasma (supplemented with NaEDTA as anticoagulant), and aliquots of 200 μl test compound dialysis solution in plasma are dispensed into the donor (plasma) chambers. Aliquots of 200 μl dialysis buffer (100 mM potassium phosphate, pH 7.4, supplemented with up to 4.7% Dextran) are dispensed into the buffer (acceptor) chamber. Incubation is carried out for 2 hours under rotation at 37° C. for establishing equilibrium.

At the end of the dialysis period, aliquots obtained from donor and acceptor chambers, respectively, are transferred into reaction tubes and processed for HPLC-MS/MS analysis. Analyte concentrations are quantified in aliquots of samples by HPLC-MS/MS against calibration curves.

Percent bound compound is calculated using the formula:

% bound=(plasma concentration−buffer concentration/plasma concentration)×100

In one embodiment the plasma protein binding of the compounds of the invention is equal to or less than 3%, preferably less than 2 percent and more preferably less than 1.5%. Exemplary values are 1.0% and 0.85%.

IP10 Production in Human Dermal MicroVascular Endothelial Cells (MVEC) after Double-Stranded DNA Stimulation

Interferon gamma-induced protein 10 (IP-10) also known as C—X—C motif chemokine ligand 10 (CXCL10) is produced as one of the responses of the presence of double-stranded DNA in the cytoplasm and resulting STING activity. In some diseases, imbalanced STING activation can result to damage in the endothelium, for example in SAVI patients (Liu Yet al. Activated STING in a vascular and pulmonary syndrome. N Engl J Med. 2014 Aug. 7; 371(6):507-518. doi: 10.1056/NEJMoa1312625). To test the efficacy of the inventive STING inhibitors, experiments in human microvascular endothelial cells (HMVEC) are performed with the inventive compounds. Dermal HMVEC are available from Lonza, US. They are cultured in 96 well plates according to the manufacturer's instructions. Using typical protocols, the cells are serum starved and then treated with the compound for 1 hour. The cells are then treated with 400 ng/mL of dsDNA as a complex with Lipofectamine 3000 (from Thermo Fisher Scientific Inc., Waltham, MA, USA) and incubated for 6 hours. The supernatants are collected and assayed for IP10 production.

Detection of IP10 is done using the U-PLEX HUMAN IP-10 ASSAY from Meso Scale Diagnostics (1601 Research Boulevard, Rockville, Maryland 20850-3173, USA) according to manufacturer's protocols.

Results: The inventive compounds show good inhibition of IP10 production after stimulation of HMVEC with dsDNA. This demonstrates that the inventive compounds show direct target engagement in MVEC cells which is not the case for some known STING inhibitors.

Table Exemplary Inhibition of Human microvascular
endothelial cells by the inventive compounds

| Example | have (IC50 nM) |
|---|---|
| 7 | 21 |
| 10 | 11 |
| 20 | 33 |

The rounded average of multiple experiments is shown.

In a preferred embodiment, the compounds of the invention for the prevention of progression or the treatment of a disease that involves undesirable STING activation in endothelial cells are those compounds, that show IC50 values of at least 0.001 nM and less than 150 nM, preferably less than 100 nM, more preferably less than 50 nM, even more preferably less than 20 nM when tested for inhibition of human dermal MVEC as described above. In another embodiment said IC 50 value of the inventive compound is in the range of and including 5 nM to and including 35 nM.

### Inhibition of STING in Fibroblasts

As many of the above-mentioned diseases like IPF or SAVI involve fibrosis, it is important to demonstrate the efficacy of the inventive compounds in fibroblast cells. In an initial test, fibroblasts from human patients suffering from SSc are stimulated with dsDNA and the response with or without the test compound is assessed, and in addition the known STING inhibitors SN-011 and H-151 ((Z. Hong et al, STING inhibitors target the cyclic dinucleotide binding pocket, Proc. Natl. Acad. Sci. U.S.A. 118 (24) e2105465118, https://doi.org/10.1073/pnas.2105465118 (2021); Haag, S. M., Gulen, M. F., Reymond, L. et al. Targeting STING with covalent small-molecule inhibitors. Nature 559, 269-273 (2018). https://doi.org/10.1038/s41586-018-0287-8).

These known inhibitors had been reported to be effective in other fibroblasts, but show very little inhibition in SSC fibroblast, while the compounds of the invention show IC50 values in the range from 3 nM to 300 nM.

In one embodiment, the compounds of the invention have an IC 50 value in human SSc fibroblasts of at least 0.1 nM to no more than 300 nM, preferably no more than 150 nM and even more preferably no more than 100 nM and most preferably no more than 80 nM.

Inhibition of IP10 production in human monocyte derived dendritic cells after cGAMP stimulation Monocyte derived dendritic cells derived from a specimen of a human donor are cultivated using standard techniques. With the exception of the respective negative controls, the cells are stimulated with cGAMP (Invivogen) in the presence or absence of different concentration of the compounds of the invention. Supernatants are collected and analysed by ELISA for IP10 presence (IP10 MSD kit, MesoScale Diagnostics). IC50 values of STING protein inhibition are calculated using standard methods. The IC50 values for the compounds of the invention are in the range and including 0.03 nM to 6.00 nM, preferably equal to or less than 4.00 nM, and more preferably equal to or less than 3.00 nM, and even more preferably equal to or less than 2.5 nM. In yet another preferred embodiment the average IC 50 value is between and including 0.07 nM and 2.10 nM. Exemplary values are 0.26 nM and 2.03 nM.

### Use in Treatment/Method of Use

As has been found, the compounds of formula (I) are characterized by their range of applications in the therapeutic field. Preferably, the compounds of the invention are used in diseases that can be treated by the inhibition of STING and/or whose progression can be prevented by the inhibition of STING.

Particular mention should be made of those applications for which the compounds of the invention are used on the basis of their pharmaceutical activity as STING inhibitors. While the cGAS/STING pathway is important for host defense against invading pathogens, such as viral infection and invasion by some intracellular bacteria, cellular stress and genetic factors may also cause production of aberrant cellular dsDNA, e.g. by nuclear or mitochondrial leakage, and thereby trigger autoinflammatory responses. Consequently, STING inhibitors have a strong therapeutic potential to be used in the treatment of diverse autoinflammatory and autoimmune diseases.

A STING inhibitor of the invention will block in full or in part inflammation and aberrant tissue remodeling in a cluster of autoimmune and inflammatory diseases including systemic lupus erythematosus (SLE), cutaneous lupus, systemic sclerosis, inflammatory bowel disease, sepsis, Sjogren's syndrome, vitiligo, prurigo nodularis, idiopathic inflammatory myopathy, myositis including dermatomyositis, rheumatoid arthritis, as well as a cluster fibrosis diseases including NASH (now referred to as MASH), IPF, chronic kidney fibrosis.

In one embodiment the inventive use of the novel STING inhibitors is to prevent or delay the progression of any of these diseases involving elevated STING activation from a milder to a more sever stage of said disease. Non-limiting examples are the progression from compensated to decompensated liver cirrhosis or the progression of chronic kidney disease from stage 2 to 3A, or 3A to 3B or from 3B to 4. In one aspect of the invention the progression of said disease is the progression of a renal disease for example but not limited to SSC renal crisis (SRC) to end stage renal disease/kidney failure, or renal death in the patient, with the use of the STING inhibitors of the invention preventing or delaying said progression.

A STING inhibitor also has applications to additional diseases such as cancer, decompensated liver cirrhosis, heart failure, AMD, retinopathy, glaucoma, diabetes, obesity, aging, muscle disorders, anti-neutrophil cytoplasm antibody (ANCA) associated vasculitis, alopecia, chronic kidney disease; Niemann-Pick Disease, Type C, myotonic dystrophy type 2, Huntington disease, Bloom syndrome, osteoarthritis, ALS, Parkinson's disease, COVID-19.
An et al., Arthritis Rheumatol. 2017 April; 69(4):800-807, disclosed that cGAS expression in peripheral blood mononuclear cells (PBMCs) was significantly higher in patients with the autoimmune disease systemic lupus erythematosus (SLE) than in normal controls. Targeted measurement of cGAMP by tandem mass spectrometry detected cGAMP in 15% of the tested SLE patients, but none of the normal or rheumatoid arthritis controls. Disease activity was higher in SLE patients with cGAMP versus those without cGAMP.
Thim-Uam et al (iScience. 2020 Sep. 4; 23(9):101530) demonstrated that STING deficiency ameliorated lupus development in Fcgr2b-deficient mice. Prabakaran et al (EBioMedicine. 2021 April; 66:103314) shows that a STING pathway inhibitor ISD017 blocks STING activity in vivo and ameliorates disease development in a mouse model for lupus. ISD017 treatment also blocks pathological cytokine responses in PBMCs from lupus patients with elevated IFN—I levels.
Skopelja-Gardner et al reported that ultraviolet B light triggers cGAS/STING-dependent skin and systemic IFN—I signature and could contribute to cutaneous lupus Alzeand fares of disease in patients with SLE (Sci Rep 2020 10:7908)
Ryu et al (Arthritis Rheumatol. 2020 November; 72(11):1905-1915) showed that plasma mtDNA concentrations were increased in the 2 Systemic sclerosis-associated interstitial lung disease (SSc-ILD) cohorts, reflective of ventilatory decline, and were positively associated with both TLR-9 and cGAS/STING activation as well as type I IFN and IL-6 expression. Liu et al (Rheumatology (Oxford) 2022 Jun. 10; keac324.) showed increased DNA leakage, STING expression and vascular inflammation in skins of SSc patients, and STING deficiency or H151 administration ameliorated fibrosis and vasculopathy both in vitro and in BLM-induced SSc mice.
Li et al show that plasma-derived DNA containing-extracellular vesicles induce STING-mediated proinflammatory responses in dermatomyositis (Theranostics. 2021; 11(15): 7144-7158). Zhou et al (J Clin Lab Anal. 2022 October; 36 (10): e24631) describes a correlation between activation of cGAS-STING pathway and myofiber atrophy/necrosis in dermatomyositis. Feng et al. suggested STING could be a potential therapeutic target in idiopathic inflammatory myositis-associated interstitial lung disease (IIM-ILD) (Feng et al., International Immunopharmacology, March 2025, 149, doi:10.1016). It was also reported that the GAS-STING pathway is activated in the muscle biopsies of idiopathic inflammatory myopathy (IIM) patients and its activation may lead to myofiber atrophy and necrosis in IIM and dermatomyositis patients (Zhou et al., J Clin Lab Anal. 2022; 36:e24631.).
Haag et al (Nature. 2018 July; 559(7713):269-273) demonstrated that a covalent STING inhibitor attenuated pathological features of autoinflammatory disease in TREX1_KO mice. Loss of function mutation of TREX1 leads rare monogenic interferonopathies such as Aicardi-Goutieres syndrome (AGS).
Hu et al (EBioMedicine. 2019 March; 41:497-508) showed that in human abdominal sepsis, STING expression was elevated in peripheral blood mononuclear cells and intestinal biopsies compared with healthy controls. In human abdominal sepsis, STING expression was elevated in peripheral blood mononuclear cells and intestinal biopsies compared with healthy controls. STING knockout mice attenuated alleviated inflammatory response, gut permeability, and decreased bacterial translocation in a sepsis model. Zeng et al (ci Transl Med. 2017 Oct. 18; 9 (412):eaan5689) also showed that STING deficiency in mice protected two sepsos modeled (LPS model and cecal ligation and puncture model) and the degree of STING expression in the human intestinal lamina propria correlated with the intestinal inflammation in septic patients. Inhibition of the ALK-STING pathway protects mice against CLP-induced polymicrobial sepsis.
In Schuliga et al., Clin. Sci. (Lond). 2020 Apr. 17; 134(7):889-905, it is described that self-DNA perpetuates IPF lung fibroblast senescence in a cGAS-dependent manner. Benmerzoug et al (Nat. Commun. 9, 1-19 (2018)) shows that STING-dependent sensing of self-DNA drives silica-induced lung inflammation, which can lead to lung fibrosis.
Additional scientific hints linking the cause for metabolic diseases such as non-alcoholic fatty liver disease (NAFLD), now referred to as metabolic dysfunction-associated steatotic liver disease (MASLD), see https://easl.eu/news/new_fatty_liver_disease_nomenclature-2, https://www.ncbi.nlm.nih.gov/pmc/articles/PMC10653297/), other fibrosing diseases such as non-alcoholic steatohepatitis (NASH), now referred to as metabolic dysfunction associated steatohepatitis (MASH), and alcoholic liver disease (ALD) with the cGAS/STING pathway have been described in Yu et al., J. Clin. Invest. 2019 Feb. 1; 129(2):546-555, and in Cho et al., Hepatology. 2018 October; 68(4): 1331-1346, and in Qiao et al., Metabolism 2018 April; 81:13-24 doi: 10.1016/j.metabol.2017.09.010. Epub 2017 Oct. 26, Petrasek et al., PNAS 2013 Oct. 8; 110(41):16544-9. doi: 10.1073/pnas.1308331110. Epub 2013 Sep. 19
Nascimento et al., Sci. Rep. 2019 Oct. 16; 9(1):14848, discloses that self-DNA release and STING-dependent sensing drives inflammation due to cigarette smoke in mice hinting at a link between the cGAS-STING pathway and chronic obstructive pulmonary disease (COPD).
Ahn et al (Cell Rep 2017 21:3873-3884) describes that STING-deficient mice protects in an Inflammatory Colitis model. Martin et al (Sci Rep 2019 Oct. 3; 9:14281) describes that STING deletion protects while or STING stimulation, exacerbates intestinal inflammation in the dextran sodium sulphate (DSS) model of colitis. These publications support STING as a potential therapeutic target for prevention of inflammatory bowel disease (IBD).
Kerur et al., Nat. Med. 2018 January; 24(1):50-61, describes that cGAS plays a significant role in noncanonical-inflammasome activation in age-related macular degeneration (AMD).
Further, the STING inhibitors also have a therapeutic potential in the treatment of cancer (see Hoong et al., Oncotarget. 2020 Jul. 28; 11(30):2930-2955, and Chen et al., Sci. Adv. 2020 Oct. 14; 6 (42):eabb8941). Furthermore shown in Bakhoum et el., Nature. 2018 Jan. 25; 553(7689):467-472: “Chromosomal instability drives metastasis through a cytosolic DNA response”, and in Liu et al., Nature. 2018 November; 563(7729):131-136: “Nuclear cGAS suppresses DNA repair and promotes tumorigenesis”.
STING inhibitors have also the potential in the treatment of obesity and diabetes as shown in Mao et al., Arterioscler Thromb Vasc Biol (2017) 37(5):920-9. doi: 10.1161/ATVBAHA.117.309017
Additionally, the STING inhibitors have also a therapeutic potential in the treatment of heart failure (King et al, Nat Med 2017 December; 23(12):1481-1487; Hu et al., Am. J. Physiol. Heart Circ. Physiol. 2020 Jun. 1; 318 (6):H1525-H1537).
Further scientific hints at a correlation between Parkinson's disease and the cGAS/STING pathway (Sliter et al., Nature. 2018 September; 561(7722):258-262), between amyotrophic lateral sclerosis (ALS) and STING (Yu et al, Cell 2020; 183:636-649) and between Sjogren's syndrome and the cGAS/STING pathway (Papinska et al., J. Dent. Res. 2018 July; 97(8):893-900) exist.
Furthermore, STING inhibitors have also a therapeutic potential in the treatment of COVID-19/SARS-CoV-2 infections as shown in Di Domizio et al., Nature. 2022 Jan. 19. doi: 10.1038/s41586-022-04421-w: “The cGAS-STING pathway drives type I IFN immunopathology in COVID-19”, and in Neufeldt et al., Commun Biol. 2022 Jan. 12; 5(1):45. doi: 10.1038/s42003-021-02983-5: “SARS-CoV-2 infection induces a pro-inflammatory cytokine response through cGAS-STING and NF-kappaB”. It has also been shown that severe COVID-19 and long COVID are associated with high expression of STING, cGAS and IFN-α (Sci Rep 2024 14:4974).
Additionally, STING inhibitors have a therapeutic potential in the treatment of renal inflammation and renal fibrosis as shown in Chung et al., Cell Metab. 2019 30:784-799: “Mitochondrial Damage and Activation of the STING Pathway Lead to Renal Inflammation and Fibrosis”, and in Maekawa et al., Cell Rep. 2019 29:1261-1273: “Mitochondrial Damage Causes Inflammation via cGAS-STING Signaling in Acute Kidney Injury”. It has also been shown that genetic deletion or pharmacological inhibition of STING ameliorates kidney inflammation fibrosis in a mouse models of chronic kidney disease (Cell Metab 2019 30:784-799).
Further, two cases of STING GOF mutants have been reported with alopecia symptom indicating STING activation can lead to alopecia (Front Immunol 2019 10:2770. doi: 10.3389; Pediatr Rheumatol Online J. 2024 22:9 doi: 10.1186). Blood mitochondrial DNA copy number has been reported as a diagnostic marker and indicator of degree of severity in alopecia areata (J Immunoassay Immunochem 2023 44:256-268).
In addition, ANCA vasculitis patients show increased levels of cGAMP and enhanced IFN—I signature. STING deficiency or a STING inhibitor protects a mouse model for ANCA associated pulmonary vasculitis (J Exp Med. 2022 219:e20220759). ANCA pulmonary vasculitis has also been reported in a SAVI patient (STING GOV mutation) (Front Immunol. 2020 11:575219).
Furthermore, the lysosomal membrane protein Niemann-Pick type C₁ (NPC1) has been identified as a cofactor in the trafficking of STING. Genetic deletion of STING significantly reduced the activation of microglia and relieved the loss of Purkinje neurons in the cerebellum of Npc1−/− mice, leading to improved motor function. This study indicates STING inhibitors as potential therapy for Niemann-Pick disease type C (Nature 2021 596(7873):570-575).
Additionally, it has been shown that in myotonic dystrophy type 2 (DM2) disease, patient PBMCs and fibroblasts show elevated type I interferon (IFN) signature which is mediated by the cGAS/STING pathway (Nat Commun. 2024 15:1534).
In Huntington's disease (HD), the mutated huntingtin gene induces DNA damage and cytosolic DNA accumulation and activates the cGAS-STING pathway to mediate inflammation and apoptosis (Proc Natl Acad Sci USA. 2024 121:e2313652121). Depletion of cGAS in HD neuron cells decreases the expression of inflammatory genes while suppressing the up-regulation of autophagy (Proc Natl Acad Sci 117:15989-15999).
In addition, Xie et al detected binding of cGAS with dsDNA in cytoplasm and the activation of the microglial cGAS-STING pathway in brains of human AD and aged mice. A STING inhibitor suppressed the activation of the cGAS-STING pathway and ameliorated AD pathogenesis in a mouse model of Alzheimer's disease (Nat Aging 2023 3:202-212).
Additionally, during ischemic stroke, tissue damage results in misplaced DNA within the cellular environment activates the cGAS/STING pathway, leading to cytokine production, neuroinflammation, and cell death (Expert Opin Drug Discov 2023 18:1133-1149; Drug Discov Today. 2023 28:103792). STING knockout decreased infarct progression, oedema volume and neuronal damage in mouse stroke model (Stroke Vasc Neurol 2023 Jul. 3:svn-2023-002320. doi: 10.1136)
Further, it has been shown that STING promotes senescence, apoptosis, and extracellular matrix degradation in osteoarthritis (Guo et al, Cell Death Dis. 2021 Jan. 4; 12(1):13. doi: 10.1038). cGAS/STING null-mice have reduced tissue inflammation, improved heart/muscle function and have an extended lifespan (Dou et al, Nature. 2017 550: 402-406). Furthermore, in humans a variation within the STING gene is associated with healthy aging, most likely due to a decreased inflammaging (Hamann et al, Gerontology 2019; 65:145-154). Taken together, a STING inhibitor will reduce senescence associated inflammation and senescent cell accumulation and will leads improvement in senescence associated diseases such as aging/muscle disorders and osteoarthritis.
Also, it was reported that the STING protein is involved in vitiligo, as the cytosolic mtDNA-cGAS-STING axis of melanocytes plays an important role in oxidative stress-triggered CD8+ T-cell response via melanocyte pyroptosis (Xu et al., Journal of Dermatological Science, 2025, 117(3), March 2025 doi:10.1016). Oxidative stress-induced mitochondrial damage in epidermal cells led to cytosolic mtDNA accumulation, which served as a trigger in activating the cGAS-STING axis in melanocytes resulting in production of IL-1 (3 and IL-18.
Prurigo nodularis is a chronic inflammatory skin condition characterized by intensely itchy pruritic nodules on the extremities and trunk that are often a result of persistent scratching. It was reported that both systemic and cutaneous immune responses in patients with PN are skewed toward a Th22/IL-22 profile (Belzberg et al., Journal of Investigative Dermatology (2021) 141, 2208e2218). Aden et al. reported that IL-22 aggravates epithelial cell death-mediated inflammation through STING activation in intestinal epithelial cells (Aden et al., J. Exp. Med. 2018 Vol. 215: 2868-2886). STING may also play a role in IL22 mediated pathogenic responses in the skin epithelium in Prurigo nodularis.

The compounds of formula (I) or (Ia), or the salts thereof for use in patients with a disease whose progression can be prevented by the inhibition of STING is an embodiment of the invention.

In one embodiment, the STING inhibitors of the invention are useful in the prevention of progression, and/or for the treatment of a condition or disease caused by immune dysregulation and involving the STING protein(s).

The use of the compounds of the invention for the prevention of progression or for the treatment of a disease or condition that involves undesirable STING activation in a manner independent of cGAS activity is one embodiment of the intervention, for examples but not limited to subjects with Niemann-Pick disease type C.

In another embodiment the compounds of the inventions are used as anti-fibrotic agents. An embodiment of the invention is the use of the compounds of the invention in the therapy of interferon-driven inflammatory and/or fibrotic diseases or symptoms, preferably those that are a side effect of an underlying disease that leads to cell damage and cytosolic DNA presence that is not derived from pathogens.

### Combinations

The compounds of formula (I) may be administered to the patient alone or in combination with one or more other pharmacologically active agents.

In a preferred embodiment of the invention the compounds may be combined with one or more pharmacologically active agents selected from the group of PDE 4 inhibitors (preferably 1-[[(5R)-2-[4-(5-chloropyrimidin-2-yl)-1-piperidyl]-5-oxo-6,7-dihydrothieno[3,2-d]pyrimidin-4-yl]amino]cyclobutyl]methanol and [1-[[(5R)-2-[4-(5-chlorophenyl-2-yl)-1-piperidyl]-5-oxo-6,7-dihydrothieno[3,2-d]pyrimidin-4-yl]amino]cyclobutyl]methanol as disclosed in WO 2013/026797), anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-limiting examples are anti-IL-23 such as Risankizumab, anti-IL-17 antibodies, anti-IL-1 antibodies, anti-IL-4 antibodies, anti-IL-13 antibodies, anti-IL-5 antibodies, anti-IL-6 antibodies such as Actemra™, anti-IL-12 antibodies and anti-IL-15 antibodies, non-specific immunotherapeutics such as interferons or other cytokines/chemokines, cytokine/chemokine receptor modulators (i.e. cytokine receptor agonists or antagonists), Toll-like receptor agonists (=TLR agonists), immune checkpoint regulators, anti-TNF antibodies for example but not limited to Humira™ and anti-B-cell activating factor (BAFF) agents e.g. without limitation Belimumab and Etanercept. Such a combination with anti-inflammatory agents and/or anti-fibrotic agents in one embodiment is a combination of one or more compounds of the invention with a) one or more known STING inhibitors and/or b) known cGAS inhibitors and/or c) anti-inflammatory agents that are not STING inhibitors and/or anti-fibrotic agents that are not STING inhibitors, for example but not limited to Pirfenidon, Nintedanib or Nerandomilast. Another aspect of the invention is to the combined use of the STIGN inhibitors of the invention in combination with known cGAS and/or STING inhibitors, for example those disclosed in the international patent applications PCT/EP2023/080705, PCT/EP2023/080711, PCT/EP2022/062496, PCT/EP2022/062480, PCT/EP2023/079890 or published as WO2021/138419, WO2023/148129, WO2023/237457, WO2024/263860, WO2025/012195 or WO2025/017045.

In a further aspect of the present invention, the one or more other pharmacologically active agents include immunosuppressive drugs, Nonsteroidal anti-inflammatory drug (NSAID), corticosteroids e.g. glucocorticoids, hydroxychloroquine or methotrexate, antibodies for example anti-B-cell activating factor (BAFF) antibody or CAR (chimeric antigen receptors) T cells.

In one aspect of the present invention, the one or more other pharmacologically active agents are RAAS inhibitors (Renin-Angiotensin-Aldosterone System). In one aspect of the present invention, the one or more other therapeutic substances is a direct renin inhibitor, an Angiotensin-Converting Enzyme (ACE) inhibitor and/or an angiotensin II receptor blocker (ARB).

In one embodiment, the invention comprise pharmaceutical compositions comprising one or more compounds of the invention and one or more other pharmacologically active agents for use in the treatment or prevention of progression of a disease selected from the group consisting of disease selected from the group consisting of systemic lupus erythematosus (SLE), cutaneous lupus, (monogenic and digenic) interferonopathies (including STING-associated vasculopathy with onset in infancy (SAVI), Aicardi-Goutieres syndrome (AGS), COPA syndrome, and familial chilblain lupus), type 1 interferonopathies with mutations in DNASE2 or ATAD3A genes, age-related macular degeneration (AMD), retinopathy, glaucoma, amyotrophic lateral sclerosis (ALS), Huntington disease, Alzheimer's disease, diabetes, obesity, inflammatory bowel disease (IBD), chronic obstructive pulmonary disease (COPD), Bloom's syndrome, Niemann-Pick Disease, Type C, ischaemic stroke, myotonic dystrophy type 2, Sjogren's syndrome, Parkinson's disease, heart failure, cancer, systemic sclerosis (SSc), vitiligo, prurigo nodularis, idiopathic inflammatory myopathy, myositis including dermatomyositis, metabolic dysfunction-associated steatotic liver disease (MASLD) (previously referred to as non-alcoholic fatty liver disease (NAFLD), metabolic dysfunction associated steatohepatitis (MASH, previously non-alcoholic steatotic hepatitis (NASH)), compensated and decompensated liver cirrhosis, acute on chronic liver failure (ACLF), alcoholic liver disease (ALD), interstitial lung disease (ILD), idiopathic pulmonary fibrosis (IPF), long COVID, aging/muscle disorders, sepsis, heart failure, anti-neutrophil cytoplasm antibody (ANCA) associated vasculitis, alopecia, chronic kidney disease, rheumatoid arthritis and osteoarthritis.

### Formulations

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, intrasternal, and subcutaneous injection or infusion. Inhalation refers to administration into the patient's lungs whether inhaled through the mouth or through the nasal passages. Topical administration includes application to the skin. The compounds of the invention may be administered via eye drops to treat Sjogren's syndrome.

Suitable forms for administration are for example tablets, capsules, solutions, syrups, emulsions or inhalable powders or aerosols. The content of the pharmaceutically effective compound(s) in each case should be in the range from 0.1 to 90 wt. %, preferably 0.5 to 50 wt. % of the total composition, i.e. in amounts which are sufficient to achieve the dosage range specified hereinafter.

The preparations may be administered orally in the form of a tablet, as a powder, as a powder in a capsule (e.g. a hard gelatin capsule), as a solution or suspension. When administered by inhalation the active substance combination may be given as a powder, as an aqueous or aqueous-ethanolic solution or using a propellant gas formulation.

Preferably, therefore, pharmaceutical formulations are characterized by the content of one or more compounds of formula (I) according to the preferred embodiments above.

It is particularly preferable if the compounds of formula (I), preferably of formula (Ia), are administered orally, and it is also particularly preferable if they are administered once or twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example kollidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatin capsules. Suitable suppositories may be made for example by mixing with carriers provided for this purpose, such as neutral fats or polyethylene glycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatin and the like. Moreover, lubricants such as magnesium stearate, sodium lauryl sulphate and talc may be used at the same time for the tableting process. In the case of aqueous suspensions, the active substances may be combined with various flavor enhancers or colorings in addition to the excipients mentioned above.

The inventive use in the prevention of and/or treatment of and/or delaying the occurrence of and/or delaying the progression of disorders is to be understood to refer to a prevention that reduces the risk for disorders related to elevated and/or deregulated STING activity, wherein prevention can be a reduction of the risk of such disorders whereby some risk may remain. Despite the use of the compounds of the invention, individual patients may still suffer from such disorders at least to some extent, although for the overall group of patients the use of the compounds of the invention typically is suitable to delay the occurrence and/or prevent such disorders.

Prevention or delay is typically identified by comparison with a control patient group or a patient not receiving any compound of the invention, preferably a patient group/patient receiving placebo and standard of care. The treatment group/patient receives standard of care for any other disorder not related to elevated and/or deregulated STING activity, and if applicable the standard of care for disorders related to elevated and/or deregulated STING activity, plus in addition one or more compound(s) of the invention.

Identification of a prevention or delay will typically require studies in a large group of patients and control group under controlled conditions, typically in a clinical trial, but the identified prevention or delay normally applies to any individual patient receiving the compound(s) of the invention, whereas the quantity of prevention or delay for the individual patient can be expected by the average value observed in the large group but modified due to individual factors. Therefore, the prevention or delay may be present but smaller than the observed average in large trials, or higher for the individual patient.

Throughout this description the term disorders is used interchangeably with diseases or conditions.

A further aspect of the present invention is to a method of preparation of a pharmaceutical composition comprising the compound of the invention for the use in the prevention of and/or treatment of and/or delaying the occurrence of and/or delaying the progression of disorders related to elevated and/or deregulated STING activity, wherein the method comprises the steps of a) producing the inventive compound or a salt thereof, preferably a pharmaceutically acceptable salt thereof, b) optionally adding with one or more inert adjuvant, diluent and/or carrier, c) optionally adding one or more pharmacologically active agents selected from the group of PDE 4 inhibitors (preferably 1-[[(5R)-2-[4-(5-chloropyrimidin-2-yl)-1-piperidyl]-5-oxo-6,7-dihydrothieno[3,2-d]pyrimidin-4-yl]amino]cyclobutyl]methanol and [1-[[(5R)-2-[4-(5-chlorophenyl-2-yl)-1-piperidyl]-5-oxo-6,7-dihydrothieno[3,2-d]pyrimidin-4-yl]amino]cyclobutyl]methanol as disclosed in WO 2013/026797), anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, preferably anti-IL-23 such as Risankizumab, anti-IL-17 antibodies, anti-IL-1 antibodies, anti-IL-4 antibodies, anti-IL-13 antibodies, anti-IL-5 antibodies, anti-IL-6 antibodies such as Actemra™, anti-IL-12 antibodies and/or anti-IL-15 antibodies, non-specific immunotherapeutics such as interferons or other cytokines/chemokines, cytokine/chemokine receptor modulators (i.e. cytokine receptor agonists or antagonists), Toll-like receptor agonists (=TLR agonists), immune checkpoint regulators, anti-TNF antibodies, preferably Humira™, and anti-BAFF agents, preferably Belimumab and/or Etanercept. Another aspect of the invention is to the combined use of one or more of the STING inhibitors of the invention in combination with known cGAS and/or STING inhibitors, and d) optionally formulating into a form for the preferred administration.

What is claimed is:

1. A compound of formula (I), 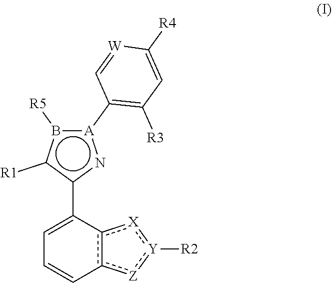
wherein
B-A is selected from the group B-A^{a} consisting of ═C—N— or —N—C═; this means A is C or N; B is C or N; but A and B are not N at the same time;
X—Y—Z is selected from the group X—Y—Z^{a} consisting of ═CH—N—N═, —N═C—NH— and —CH₂—N—C(O)—;
W is selected from the group W^{a} consisting of ═CH— and =N—;
R¹ is selected from the group R^{1a} consisting of R6-C₁₋₅-alkyl- and C₃₋₆-cycloalkyl-;
R² is selected from the group R²¹ consisting of; 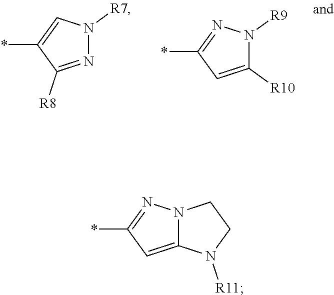
wherein * denotes the attachment point R2 in formula (I);
R³ is selected from the group R^{3a} consisting of halogen, HO—, C₁₋₃-alkyl-, C₁₋₅-alkyl-O—, C₁₋₃-alkyl-O—C₁₋₃-alkyl-, C₃₋₅-alkenyl-O—, C₂₋₃-alkenyl-O—C₁₋₃-alkyl-C₃₋₆-cycloalkyl- and heterocyclyl-;
wherein a C₁₋₃-alkyl-group is optionally substituted with 1 to 3 selected from the group consisting of fluorine, HO—, H₃C—O—, F₃C—O—, and F2HC—O—;
wherein the C₁₋₅-alkyl-group of the C₁₋₅-alkyl-O-group is optionally substituted with 1 to 5 (e.g. 2, 3 or 4) substituents independently selected from the group consisting of fluorine, HO—, H₂N—C(O)—, C₃₋₄-cycloalkyl-, C₁₋₃-alkyl-O—, heterocyclyl and heteroaryl;
R⁴ is selected from the group R⁴¹ consisting of C₁₋₃-alkyl-S(O)₂—, C₁₋₃-alkyl-S(O)—, (C₁₋₃-alkyl)₂P(O)—, (C₁₋₃-alkyl)(C₃₋₆-cycloalkyl)P(O)—, —C(O)—, H₂N—C(O)—, C₁₋₃-alkyl-NH—C(O)—, (C₁₋₃-alkyl)₂N—C(O)— and 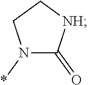 [SMILES: *N1CCNC1=O]
wherein * denotes the attachment point of R^{4a};
R⁵ is selected from the group R^{5a} consisting of C₁₋₄-alkyl-;
R⁶ is selected from the group R^{6a} consisting of H—, HO—and Halogen;
R⁷ is selected from the group R^{7a} consisting of H—, Halogen, HO—, HO(C₁₋₆-alkyl)₂C—CH₂—, C₁₋₃-alkyl-O—, C₁₋₆-alkyl-, C₃₋₅-alkenyl-, C₃₋₆-cycloalkyl-, aryl, heteroaryl and heterocyclyl;
wherein the C₁₋₆-alkyl-group is optionally substituted with 1 to 3 substituents independently selected from the group consisting of Halogen, HO—, and C₁₋₃-alkyl-O—,
wherein the heteroaryl group is optionally substituted with 1 substituent selected from the group consisting of C₁₋₃-alkyl-,
wherein the C₃₋₆-cycloalkyl- and/or aryl group is optionally substituted with 1 to 3 substituents independently selected from the group consisting of (H₃C)₂N—C(O)—;
R⁸ is selected from the group R^{8a} consisting of H—, C₁₋₆-alkyl-O—and heterocyclyl-O—;
wherein the C₁₋₆-alkyl-O-group is optionally substituted with 1 substituent selected from the group consisting of C₁₋₃-alkyl-O—and (H₃C)₂N—C(O)—;
C₁₋₅-alkyl-O—and Halogen;
R⁹ is selected from the group R^{9a} consisting of H—, C₁₋₃-alkyl- and H₂N—C(O)—CH₂—;
R¹⁰ is selected from the group R^{10a} consisting of C₁₋₃-alkyl-, C₂₋₃-alkenyl-, C₁₋₃-alkyl-O—, C₁₋₃-alkyl-S—and C₃₋₄-cycloalkyl-,
wherein the C₁₋₃-alkyl-group is optionally substituted with 1 to 3 substituents selected from the group consisting of F—;
R¹¹ is selected from the group R^{11a} consisting of C₁₋₅-alkyl-, or —C₁₋₅-alkyl-C(O)—, wherein the C₁₋₅-alkyl-, group is optionally substituted independently of one another with 1 to 3 substituents selected from the group consisting of C₁₋₃-alkyl-, halogen and HO—;
or a salt thereof.

2. A compound according to claim 1, wherein
B-A is selected from the group B-A^{c} consisting of —N—C═; this means A is C; B is N, or a salt thereof.

3. A compound according to claim 1, wherein
W is selected from the group W^{b} consisting of ═CH—,
or a salt thereof.

4. A compound according to claim 1, wherein
X—Y—Z is selected from the group X—Y—Z^{b} consisting of ═CH—N—N═ and —N═C—NH—, or a salt thereof.

5. A compound according to claim 1, wherein
R² is selected from the group R^{2b} consisting of, 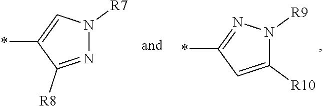
wherein * denotes the attachment points to the core structure,
or a salt thereof.

6. A compound according to claim 1, wherein
R³ is selected from the group R^{3c} consisting of F—, Cl—, HO—, H₃C—, F₃C—, (H₃C)₂C—, H₃C—CH₂—, cyclopropyl-, H₃C—O—, FH₂C—O—, F₂HC—O—, F₃C—O—, (H₃C)₂CH—O—, (H₃C)₃C—O—, H₃C—CH₂—O—, F₂HC—CH₂—O—, F₂C(CH₃)—CH₂—O—, H₃C—CH₂—CH₂—O—, F—CH₂—CH₂—O—, HO—CH₂—CH₂—O—, H₂N—CH₂—C(O)—, H₂C═CH—CH₂—O—, H₃C—O—CH₂—CH₂—O—, H₃C—O—CH₂—CH₂—CH₂—O—, (H₃C)₂C(OH)—CH₂—CH₂—O—, (H₃C)₂CH—CH₂—O—, cyclopropyl-O—, cyclopropyl-CH₂—O—, H₃C—O—CH₂—CH₂—, H₃C—O—CH₂—, 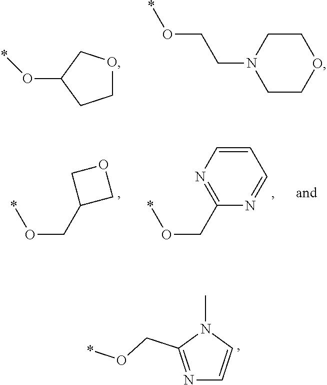
wherein * denotes the attachment point to the core structure,
or a salt thereof.

7. A compound according to claim 1, wherein
R⁴ is selected from the group R^{4b} consisting of
(C₁₋₃-alkyl)₂P(O)—, C₁₋₃-alkylNH—C(O)—, (C₁₋₃-alkyl)₂N—C(O)— and H₂N—C(O)—, or a salt thereof.

8. A compound according to claim 1, wherein the compound of formula (I) is a compound of formula (Ia) 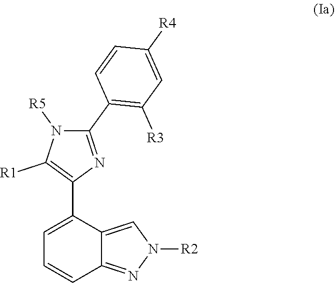
or a salt thereof.

9. A compound according to claim 1, wherein
B-A is selected from the group B-A^{c} consisting of —N—C═; this means A is C; B is N;
W is selected from the group W^{b} consisting of ═CH—;
X—Y—Z is selected from the group X—Y—Z^{b} consisting of ═CH—N—N═ and —N═C—NH—;
R¹ is selected from the group R^{1b} consisting of isopropyl- and cyclopropyl-;
R² is selected from the group R^{2a} consisting of
R² is selected from the group R^{2a} consisting of 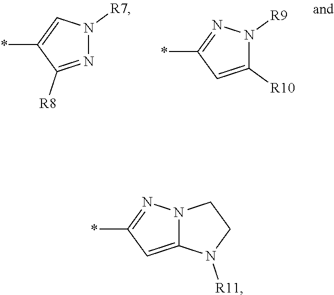
wherein * denotes the attachment point to the core;
R³ is selected from the group R^{3c} consisting of F—, Cl—, HO—, H₃C—, F₃C—, (H₃C)₂C—, H₃C—CH₂—, cyclopropyl-, H₃C—O—, FH₂C—O—, F₂HC—O—, F₃C—O—, (H₃C)₂CH—O—, (H₃C)₃C—O—, H₃C—CH₂—O—, F₂HC—CH₂—O—, F₂C(CH₃)—CH₂—O—, H₃C—CH₂—CH₂—O—, F—CH₂—CH₂—O—, HO—CH₂—CH₂—O—, H₂N—CH₂—C(O)—, H₂C═CH—CH₂—O—, H₃C—O—CH₂—CH₂—O—, H₃C—O—CH₂—CH₂—CH₂—O—, (H₃C)₂C(OH)—CH₂—CH₂—O—, (H₃C)₂CH—CH₂—O—, cyclopropyl-O—, cyclopropyl-CH₂—O—, H₃C—O—CH₂—CH₂—, H₃C—O—CH₂—, 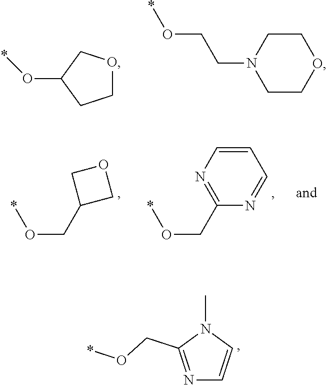
wherein * denotes the attachment point,
R⁴ is selected from the group R^{4b} consisting of (C₁₋₃-alkyl)₂P(O)—, C₁₋₃-alkylNH—C(O)—, (C₁₋₃-alkyl)₂N—C(O)— and H₂N—C(O)—;
R⁵ is selected from the group R^{5b} consisting of H₃C—, H₃C—CH₂—, H₃C—CH₂—CH₂—and (H₃C)₂C—;
R⁷ is selected from the group R⁷¹ consisting of H—, Halogen, HO—, HO(C₁₋₆-alkyl)₂C—CH₂—, C₁₋₃-alkyl-O—, C₁₋₆-alkyl-, C₃₋₅-alkenyl-, C₃₋₆-cycloalkyl-, aryl, heteroaryl and heterocyclyl;
wherein the C₁₋₆-alkyl-group is optionally substituted with 1 to 3 substituents independently selected from the group consisting of Halogen, HO—, and C₁₋₃-alkyl-O—,
wherein the heteroaryl group is optionally substituted with 1 substituent selected from the group consisting of C₁₋₃-alkyl-,
wherein the C₃₋₆-cycloalkyl- and/or aryl group is optionally substituted with 1 to 3 substituents independently selected from the group consisting of (H₃C)₂N—C(O)—;
R⁸ is selected from the group R^{8a} consisting of H—, C₁₋₆-alkyl-O—and heterocyclyl-O—;
wherein the C₁₋₆-alkyl-O-group is optionally substituted with 1 substituent selected from the group consisting of C₁₋₃-alkyl-O—and (H₃C)₂N—C(O)—;
C₁₋₅-alkyl-O—and Halogen;
R⁹ is selected from the group R^{9a} consisting of H—, C₁₋₃-alkyl- and H₂N—C(O)—CH₂—;
R¹⁰ is selected from the group R^{10a} consisting of C₁₋₃-alkyl-, C₂₋₃-alkenyl-, C₁₋₃-alkyl-O—, C₁₋₃-alkyl-S—and C₃₋₄-cycloalkyl-,
wherein the C₁₋₃-alkyl-group is optionally substituted with 1 to 3 substituents selected from the group consisting of F—;
R¹¹ is selected from the group R^{11a} consisting of C₁₋₅-alkyl-, or —C₁₋₅-alkyl-C(O)—, wherein the C₁₋₅-alkyl-, group is optionally substituted independently of one another with 1 to 3 substituents selected from the group consisting of C₁₋₃-alkyl-, halogen and HO—;
or a salt thereof, optionally a pharmaceutically acceptable salt.

10. A compound according to claim 1, wherein
B-A is selected from the group B-A^{c} consisting of —N—C═; this means A is C; B is N;
W is selected from the group W^{b} consisting of ═CH—;
X—Y—Z is selected from the group X—Y—Z^{c} consisting of ═CH—N—N═;
R¹ is selected from the group R^{1c} consisting of cyclopropyl-;
R² is selected from the group R^{2d} consisting of 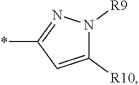
wherein * denotes the attachment point;
R³ is selected from the group R^{3e} consisting of H₃C—, F₂HC—CH₂—O—, F₃C—O—, and H₃C—O—CH₂—,
R⁴ is selected from the group R^{4d} consisting of (CH₃CH₂)₂P(O)—, (CH₃)(CH₃CH₂)P(O)—and (CH₃)₂P(O)—;
R⁵ is selected from the group R^{5b} consisting of H₃C—, H₃C—CH₂—, H₃C—CH₂—CH₂—and (H₃C)₂C—;
R⁹ is selected from the group R^{9d} consisting of H₃C—, or from group R^{9c} consisting of H—;
R¹⁰ is selected from the group R^{10 c} consisting of cyclopropyl-CF₂H—and CF₃—;
or a salt thereof, optionally a pharmaceutically acceptable salt.

11. A compound according to claim 1, selected from the following examples:

| No. | Structure |
|---|---|
| I | 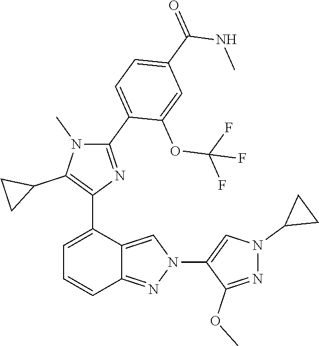 [SMILES: CNC(=O)c1ccc(-c2nc(-c3cccc4nn(-c5cn(C6CC6)nc5OC)cc34)c(C3CC3)n2C)c(OC(F)(F)F)c1] |
| II | 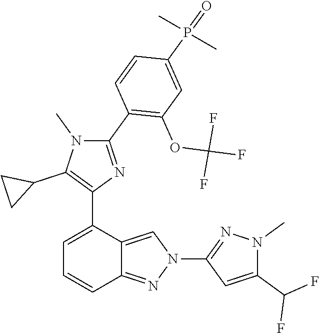 [SMILES: Cn1nc(-n2cc3c(-c4nc(-c5ccc(P(C)(C)=O)cc5OC(F)(F)F)n(C)c4C4CC4)cccc3n2)cc1C(F)F] |
| III | 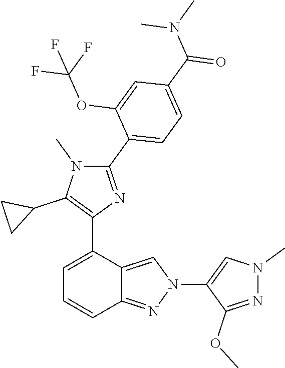 [SMILES: COc1nn(C)cc1-n1cc2c(-c3nc(-c4ccc(C(=O)N(C)C)cc4OC(F)(F)F)n(C)c3C3CC3)cccc2n1] |
| IV | 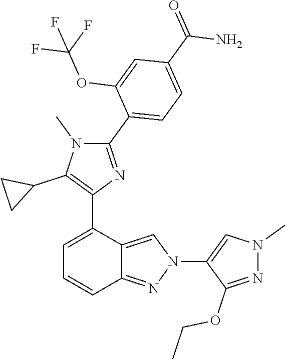 [SMILES: CCOc1nn(C)cc1-n1cc2c(-c3nc(-c4ccc(C(N)=O)cc4OC(F)(F)F)n(C)c3C3CC3)cccc2n1] |
| XI | 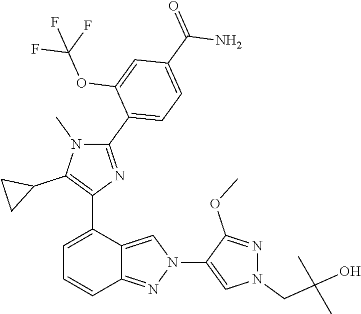 [SMILES: COc1nn(CC(C)(C)O)cc1-n1cc2c(-c3nc(-c4ccc(C(N)=O)cc4OC(F)(F)F)n(C)c3C3CC3)cccc2n1] |
| XII | 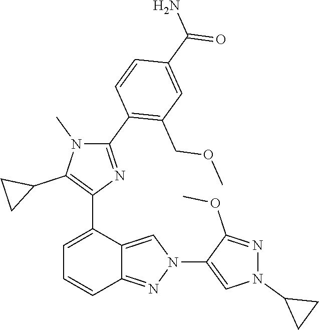 [SMILES: COCc1cc(C(N)=O)ccc1-c1nc(-c2cccc3nn(-c4cn(C5CC5)nc4OC)cc23)c(C2CC2)n1C] |
| XIII | 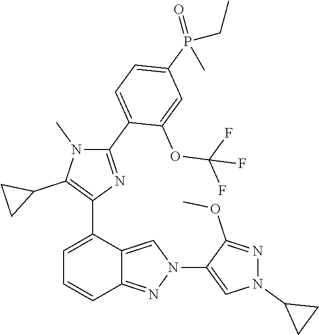 [SMILES: CCP(C)(=O)c1ccc(-c2nc(-c3cccc4nn(-c5cn(C6CC6)nc5OC)cc34)c(C3CC3)n2C)c(OC(F)(F)F)c1] |
| XIV | 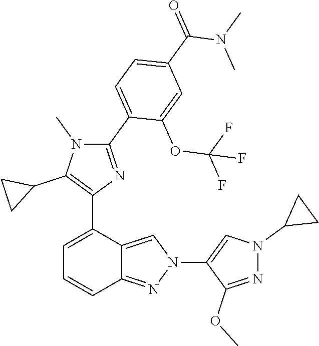 [SMILES: COc1nn(C2CC2)cc1-n1cc2c(-c3nc(-c4ccc(C(=O)N(C)C)cc4OC(F)(F)F)n(C)c3C3CC3)cccc2n1] |
| V | 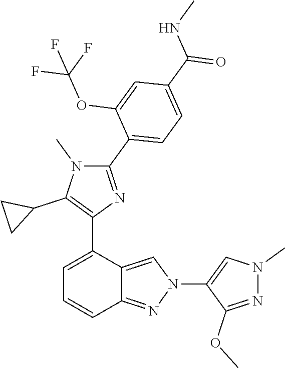 [SMILES: CNC(=O)c1ccc(-c2nc(-c3cccc4nn(-c5cn(C)nc5OC)cc34)c(C3CC3)n2C)c(OC(F)(F)F)c1] |
| VI | 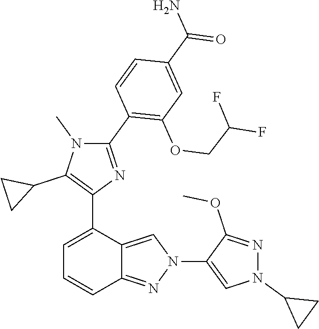 [SMILES: COc1nn(C2CC2)cc1-n1cc2c(-c3nc(-c4ccc(C(N)=O)cc4OCC(F)F)n(C)c3C3CC3)cccc2n1] |
| VII | 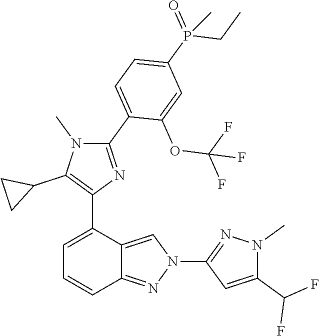 [SMILES: CCP(C)(=O)c1ccc(-c2nc(-c3cccc4nn(-c5cc(C(F)F)n(C)n5)cc34)c(C3CC3)n2C)c(OC(F)(F)F)c1] |
| VIII | 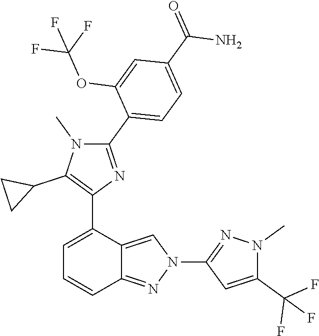 [SMILES: Cn1nc(-n2cc3c(-c4nc(-c5ccc(C(N)=O)cc5OC(F)(F)F)n(C)c4C4CC4)cccc3n2)cc1C(F)(F)F] |
| XV | 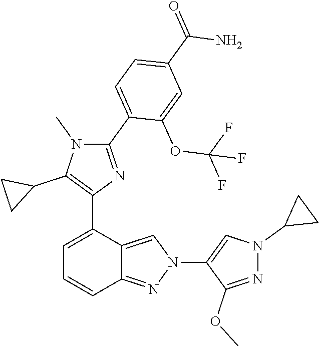 [SMILES: COc1nn(C2CC2)cc1-n1cc2c(-c3nc(-c4ccc(C(N)=O)cc4OC(F)(F)F)n(C)c3C3CC3)cccc2n1] |
| XVI | 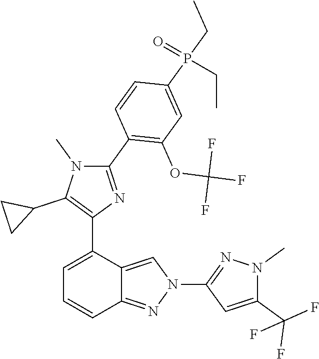 [SMILES: CCP(=O)(CC)c1ccc(-c2nc(-c3cccc4nn(-c5cc(C(F)(F)F)n(C)n5)cc34)c(C3CC3)n2C)c(OC(F)(F)F)c1] |
| XVII | 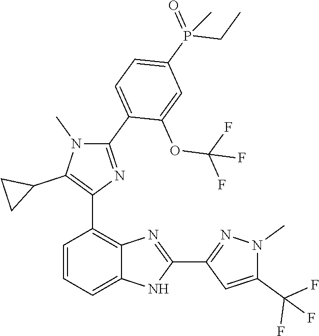 [SMILES: CCP(C)(=O)c1ccc(-c2nc(-c3cccc4[nH]c(-c5cc(C(F)(F)F)n(C)n5)nc34)c(C3CC3)n2C)c(OC(F)(F)F)c1] |
| XVIII | 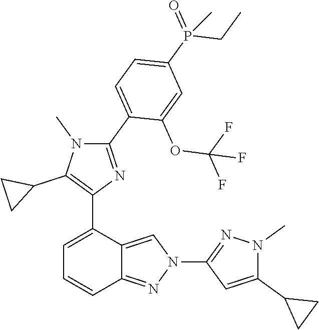 [SMILES: CCP(C)(=O)c1ccc(-c2nc(-c3cccc4nn(-c5cc(C6CC6)n(C)n5)cc34)c(C3CC3)n2C)c(OC(F)(F)F)c1] |
| IX | 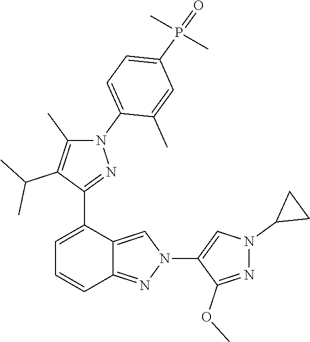 [SMILES: COc1nn(C2CC2)cc1-n1cc2c(-c3nn(-c4ccc(P(C)(C)=O)cc4C)c(C)c3C(C)C)cccc2n1] |
| X | 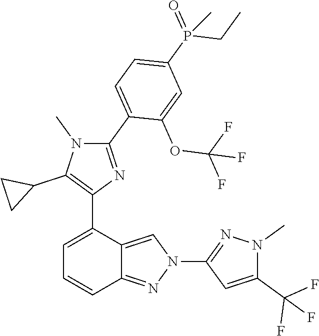 [SMILES: CCP(C)(=O)c1ccc(-c2nc(-c3cccc4nn(-c5cc(C(F)(F)F)n(C)n5)cc34)c(C3CC3)n2C)c(OC(F)(F)F)c1] |
| XXI | 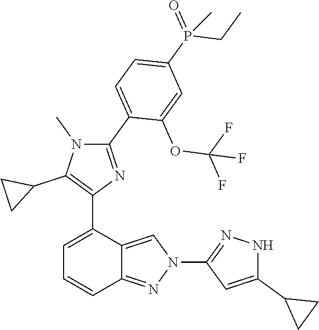 [SMILES: CCP(C)(=O)c1ccc(-c2nc(-c3cccc4nn(-c5cc(C6CC6)[nH]n5)cc34)c(C3CC3)n2C)c(OC(F)(F)F)c1] |
| XXIII | 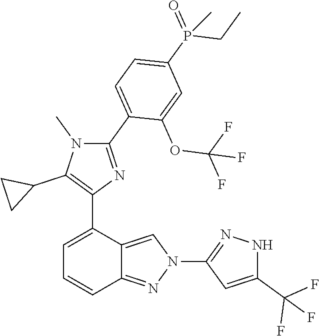 [SMILES: CCP(C)(=O)c1ccc(-c2nc(-c3cccc4nn(-c5cc(C(F)(F)F)[nH]n5)cc34)c(C3CC3)n2C)c(OC(F)(F)F)c1] |
| XIX | 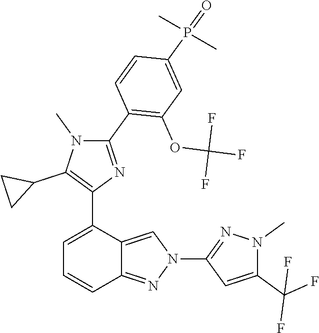 [SMILES: Cn1nc(-n2cc3c(-c4nc(-c5ccc(P(C)(C)=O)cc5OC(F)(F)F)n(C)c4C4CC4)cccc3n2)cc1C(F)(F)F] |
| XX | 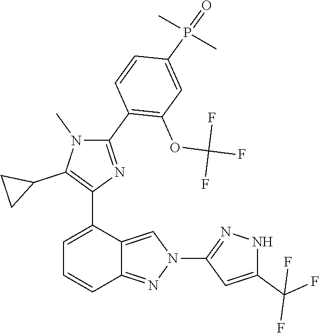 [SMILES: Cn1c(-c2ccc(P(C)(C)=O)cc2OC(F)(F)F)nc(-c2cccc3nn(-c4cc(C(F)(F)F)[nH]n4)cc23)c1C1CC1] |
| XXII | 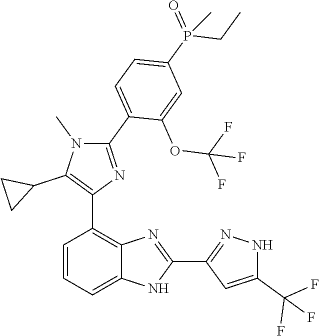 [SMILES: CCP(C)(=O)c1ccc(-c2nc(-c3cccc4[nH]c(-c5cc(C(F)(F)F)[nH]n5)nc34)c(C3CC3)n2C)c(OC(F)(F)F)c1] |
| XXIV | 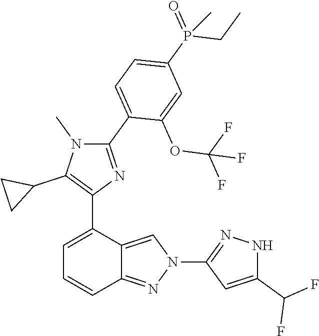 [SMILES: CCP(C)(=O)c1ccc(-c2nc(-c3cccc4nn(-c5cc(C(F)F)[nH]n5)cc34)c(C3CC3)n2C)c(OC(F)(F)F)c1] |
| XXV | 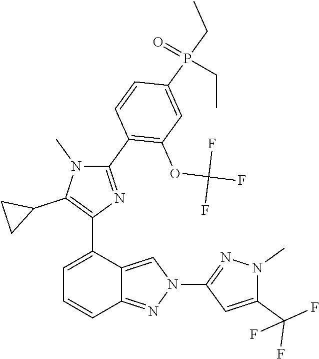 [SMILES: CCP(=O)(CC)c1ccc(-c2nc(-c3cccc4nn(-c5cc(C(F)(F)F)n(C)n5)cc34)c(C3CC3)n2C)c(OC(F)(F)F)c1] |
| XXVII | 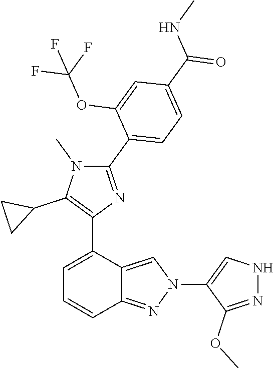 [SMILES: CNC(=O)c1ccc(-c2nc(-c3cccc4nn(-c5c[nH]nc5OC)cc34)c(C3CC3)n2C)c(OC(F)(F)F)c1] |
| XXIX | 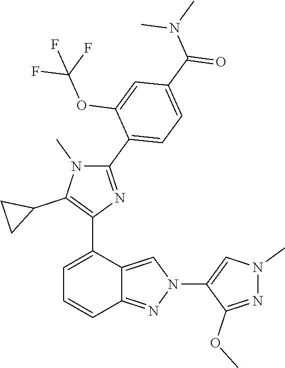 [SMILES: COc1nn(C)cc1-n1cc2c(-c3nc(-c4ccc(C(=O)N(C)C)cc4OC(F)(F)F)n(C)c3C3CC3)cccc2n1] |
| XXVI | 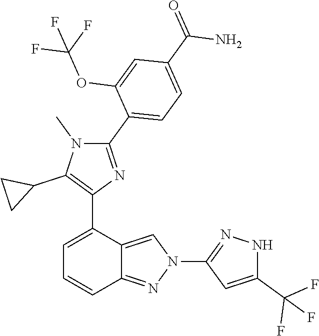 [SMILES: Cn1c(-c2ccc(C(N)=O)cc2OC(F)(F)F)nc(-c2cccc3nn(-c4cc(C(F)(F)F)[nH]n4)cc23)c1C1CC1] |
| XXVIII | 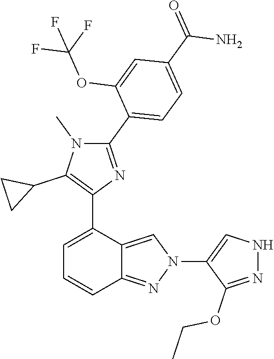 [SMILES: CCOc1n[nH]cc1-n1cc2c(-c3nc(-c4ccc(C(N)=O)cc4OC(F)(F)F)n(C)c3C3CC3)cccc2n1] |
| XXX | 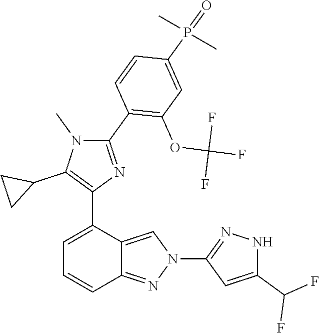 [SMILES: Cn1c(-c2ccc(P(C)(C)=O)cc2OC(F)(F)F)nc(-c2cccc3nn(-c4cc(C(F)F)[nH]n4)cc23)c1C1CC1] |

12. A compound according to claim 1, selected from the group consisting of examples 1 to 22.

13. A salt, optionally a pharmaceutically acceptable salt, of any of the compounds of claim 11.

14. A method of treating a disease that can be treated by the inhibition of STING, said method comprising administering to a patient in need thereof a compound of formula (I) according to claim 1 or a salt thereof.

15. The method of claim 14 wherein the disease is selected from the group consisting of systemic lupus erythematosus (SLE), cutaneous lupus, (monogenic and digenic) interferonopathies (including STING-associated vasculopathy with onset in infancy (SAVI), Aicardi-Goutieres syndrome (AGS), COPA syndrome, and familial chilblain lupus), type 1 interferonopathies with mutations in DNASE2 or ATAD3A genes, age-related macular degeneration (AMD), retinopathy, glaucoma, amyotrophic lateral sclerosis (ALS), Huntington disease, Alzheimer's disease, diabetes, obesity, inflammatory bowel disease (IBD), chronic obstructive pulmonary disease (COPD), Bloom's syndrome, Niemann-Pick Disease, Type C, ischaemic stroke, myotonic dystrophy type 2, Sjogren's syndrome, Parkinson's disease, heart failure, cancer, systemic sclerosis (SSc), vitiligo, prurigo nodularis, idiopathic inflammatory myopathy, myositis including dermatomyositis, metabolic dysfunction-associated steatotic liver disease (MASLD) (previously referred to as non-alcoholic fatty liver disease (NAFLD)), metabolic dysfunction associated steatohepatitis (MASH, previously non-alcoholic steatotic hepatitis (NASH)), compensated and decompensated liver cirrhosis, acute on chronic liver failure (ACLF), alcoholic liver disease (ALD), interstitial lung disease (ILD), idiopathic pulmonary fibrosis (IPF), long COVID, aging/muscle disorders, sepsis, heart failure, anti-neutrophil cytoplasm antibody (ANCA) associated vasculitis, alopecia, chronic kidney disease, rheumatoid arthritis and osteoarthritis.

16. A pharmaceutical composition comprising at least one compound of formula (I) according to claim 1 and/or a salt thereof, and optionally one or more pharmaceutically acceptable carriers and/or excipients.

17. A salt, optionally a pharmaceutically acceptable salt, of any of the compounds of claim 12.
